(19) United States
(12) Patent Application Publication
(10) Pub. No.: US 2026/0224399 A1
(43) Pub. Date: Aug. 6, 2026

(54) DRUG-CONTAINING DEVICES, SUPRACHOROIDAL SPACE IMPLANTS, AND ADAPTERS FOR INJECTION

(71) Applicant: BEJING SIGHTNOVO MEDICAL TECHNOLOGY CO., LTD, Beijing (CN)

(72) Inventors: Chan ZHAO, Beijing (CN); Chuan LI, Beijing (CN); Chaoran XIA, Beijing (CN); Yueguang SUN, Beijing (CN)

(21) Appl. No.: 19/125,194

(22) PCT Filed: Nov. 10, 2023
(86) PCT No.: PCT/CN2023/130900

§ 371 Date: Apr. 28, 2025

(30) Foreign Application Priority Data
Nov. 11, 2022 (WO) PCT/CN2022/131454

(51) Int. Cl. A61F 9/007; A61K 31/573; A61K 45/06; A61K 47/02; A61K 47/36; A61M 5/31; A61M 5/315; A61M 5/32

(52) U.S. Cl. CPC A61F 9/00736; A61K 31/573; A61K 45/06; A61K 47/02; A61K 47/36; A61M 5/31566; A61M 5/3158; A61M 5/3287; A61M 2005/3143; A61M 2205/0216

(57) ABSTRACT

A pre-filled syringe for injecting a drug composition into an eye comprises: a syringe barrel (1) comprising a proximal end and a distal end; a floating seal (3) in the syringe barrel (1); a needle base proximal to the floating seal (3), wherein the floating seal (3) and the needle base elastically engage each other; a drug composition, wherein the drug composition is contained in a lumen (7) formed by the floating seal (3) and the distal end of the syringe barrel (1); a needle (6) for insertion into the eye, the needle (6) comprising: (i) a needle proximal end engaging the needle base; (ii) a needle distal end; (iii) a needle distal opening (6a); (iv) a needle body opening (6b) between the needle proximal end and the needle distal end, wherein the needle body opening (6b) is proximal to the needle distal opening (6a); and (v) a needle body passageway connecting the needle distal opening (6a) and the needle body opening (6b), wherein the needle base is configured to advance the needle (6) distally toward and/or through the floating seal (3).

### CROSS-REFERENCE TO RELATED APPLICATIONS

This application claims priority to International Application No. PCT/CN2022/131454, filed on Nov. 11, 2022, which are incorporated herein by reference in their entireties.

### FIELD

The present disclosure in some aspects relates to the field of medical device and apparatus, and specifically a device, kit, assembly, or system for medical penetration and drainage.

### BACKGROUND

In existing methods of treatment involving the suprachoroidal space (SCS), a syringe is typically used to inject a medication into the SCS. When performing the puncture, the punctuation depth of a needle of the syringe needs to be manually controlled, and a medical personnel has to rely on his or her experience to determine if the needle has entered the SCS. However, the eye structure of different patients usually vary from each other and the determination of needle depth by the medical personnel may not be accurate. As a result, the precise placement of the needle relative to the SCS cannot be guaranteed. Furthermore, when injecting the medication, the plunger of the syringe has to be constantly pressed manually. Skillful operation by the medical personnel is required in order to stabilize the injection speed and prevent fluctuations in the flow speed. However, in practice, it is challenging to guarantee steady injection every time. Therefore, improved devices and methods for medical penetration such as injection into the SCS are needed.

In the meanwhile, therapeutic agents for treatment involving SCS are required to have a high level of sterility, and be injected with minimal dosage errors. Therefore, a need exists for developing injection device or systems that not only achieve precise injection into SCS, but also minimizes contaminations from the environment, eliminates dosage errors, and adds convenience for medical personnel.

The present disclosure addressed these and other needs by providing a pre-loaded or pre-filled injection device that comprises a device suitable for precise injection into SCS, and one or more therapeutic agents.

### SUMMARY

In some embodiments, provided herein is a pre-filled syringe for injecting a drug composition into an eye, comprising: a syringe barrel comprising a proximal end and a distal end; a floating seal in the syringe barrel; a needle base that is proximal to or provided on the floating seal; a drug composition contained in a lumen formed by the floating seal and an end of the syringe barrel; a needle for insertion into the eye, the needle comprising: (i) a needle proximal end engaging the needle base; (ii) a needle distal end; and (iii) a needle distal opening, wherein the needle base is configured to advance the needle distally toward a tissue of a subject.

In some embodiments, provided herein is a pre-filled syringe for injecting a drug composition into an eye, comprising: a syringe barrel comprising a proximal end and a distal end; a floating seal in the syringe barrel; a needle base proximal to the floating seal, wherein the floating seal and the needle base elastically engage each other; a drug composition contained in a lumen formed by the floating seal and the distal end of the syringe barrel; a needle for insertion into the eye, the needle comprising: (i) a needle proximal end engaging the needle base; (ii) a needle distal end; (iii) a needle distal opening; (iv) a needle body opening between the needle proximal end and the needle distal end, wherein the needle body opening is proximal to the needle distal opening; and (v) a needle body passageway connecting the needle distal opening and the needle body opening, wherein the needle base is configured to advance the needle distally toward and/or through the floating seal.

In any of the embodiments herein, the drug composition can comprise a formulation of triamcinolone. In some embodiments, the formulation of triamcinolone comprises: (i) triamcinolone or a pharmaceutically acceptable salt thereof; (ii) hyaluronic acid or a pharmaceutically acceptable derivative, analog, salt, or solvate thereof; (iii) one or more buffer agents; and (iv) one or more tonicity agents.

In some embodiments, provided herein is a method of placing a stent in an eye, comprising: (a) inserting a needle at an injection site of the eye between the sclera and the choroid of the eye; (b) delivering a flowable composition through the needle to form a suprachoroidal space; (c) removing the needle from the eye; and (d) positioning a stent into the suprachoroidal space through the injection site. In some embodiments, the flowable composition comprises a viscoelastic material. In some embodiments, the injection site is expanded before a stent is implanted into the suprachoroidal space through the expanded injection site. In some embodiments, the stent is positioned in the suprachoroidal space on a plane that is parallel to the equator of the eye ball. In some embodiments, the stent implanted in the suprachoroidal space is configured to maintain the suprachoroidal space in an expanded state compared to the state prior to delivering the flowable composition into the eye, and the expanded state is maintained for at least 4 months, 8 months, 12 months, 18 months, 24 months, 30 months, 36 months, or longer.

In some embodiments, provided herein is a set of adapters for a syringe, comprising: a contact member extending from a proximal end to a distal end; and a pressing unit comprising a first elastic element; wherein the syringe comprises a syringe barrel extending from a proximal end to a distal end and forming a lumen extending from the proximal end to the distal end; a push shaft extending from a proximal end to a distal end and forming a seal between the distal end of the push shaft and the syringe barrel; and a needle extending from a proximal end to a distal end comprising an end opening for the fluid to flow from the lumen and through the distal end of the syringe barrel via a needle base; wherein the contact member can be installed to the needle distal end of the syringe such that the distal end of the contact member is distal to the needle distal end opening, and the distal end of the contact member can directly contact with surface tissues at a target injection site; and wherein the pressing unit can be installed to the syringe barrel and/or push shaft of the syringe such that the pressing unit elastically engages the push shaft and/or the syringe barrel of the syringe via the first elastic element.

In some embodiments, the set of adapters further comprise a second elastic element configured to be installed between the contact member and the needle base, and wherein the second elastic element elastically connects the proximal end of the contact member to the needle base. In some embodiments, the second elastic element is a spring or an elastic sheath.

In some embodiments, the set of adapters can be used in conjunction with any of the syringes disclosed herein, including any pre-filled syringes disclosed herein.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1E show schematic diagrams of the different stages of operating an exemplary medical puncturing device, for example, during the punctuation and injection into a suprachoroidal space (SCS) 14. FIG. 1F show steps of operating an exemplary medical puncturing device without a contact member (e.g., 1b shown in FIGS. 1A-1E), where a distal seal (e.g., 8 shown in FIGS. 1A-1E) may directly contact a tissue.

FIGS. 2A-2E show schematic diagrams of the different stages of operating an exemplary medical puncturing device, for example, during the punctuation and injection into a suprachoroidal space (SCS) 14. FIG. 2F show steps of operating an exemplary medical puncturing device without a contact member (e.g., 1b shown in FIGS. 2A-2E), where a distal seal (e.g., 8 shown in FIGS. 2A-2E) may directly contact a tissue. FIG. 2G shows steps of operating an exemplary medical puncturing device comprising an additional actuation member 2′ engaging floating seal 3 via another spring 4′, whereas actuation member 2 engages floating seal 3 via spring 4.

FIGS. 3A-3F are partial structure diagrams of exemplary medical puncturing devices comprising floating seal 3 and one or more needle body openings (6b or 6b1, 6b2, and/or 6b3) and needle distal opening 6a.

FIGS. 4A-4C are partial structure diagrams of exemplary medical puncturing devices comprising floating seal 3 and needle body opening 6b.

FIGS. 5A-5F are partial structure diagrams of exemplary medical puncturing devices comprising floating seals 3a and 3b and one or more needle body openings (6b or 6b1 and/or 6b2).

FIG. 6 shows a partial structure diagram of an exemplary medical puncturing device comprising a through angled guiding groove 3a and one-way valve 9.

FIG. 7 shows a partial structure diagram of an exemplary medical puncturing device comprising a through angled guiding groove 3a and one-way valve 9.

FIG. 8 shows a partial structure diagram of an exemplary medical puncturing device comprising a non-through angled guiding groove 3a.

FIG. 9 shows a partial structure diagram of an exemplary medical puncturing device comprising an angled guiding needle hole 6c and one-way valve 9.

FIG. 10 shows a partial structure diagram of an exemplary medical puncturing device comprising an angled guiding needle hole 6c and needle hole plug 10.

FIGS. 11A-11B show schematic diagrams of implanting catheter 11 into SCS 14 using an exemplary medical apparatus assembly comprising a central guiding groove 2c. FIG. 11A shows contact member 1b that contacts a tissue, while FIG. 11B shows distal seal 8 that contacts a tissue without an intervening contact member.

FIGS. 12A-12C show schematic diagrams of the different stages of operating an exemplary medical puncturing device.

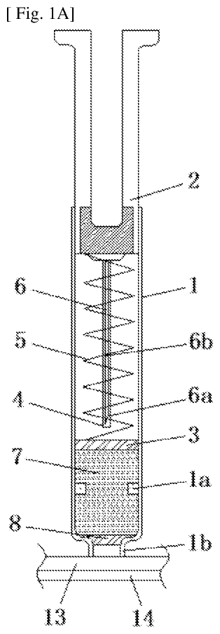

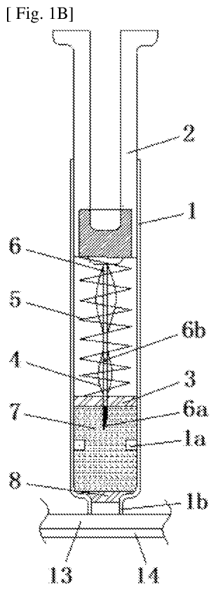

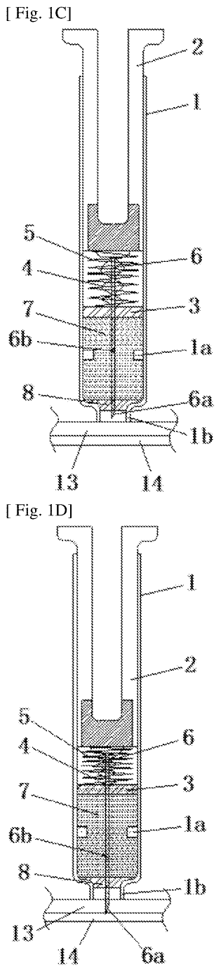

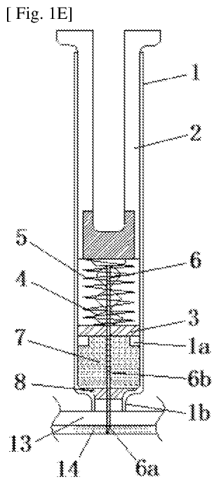

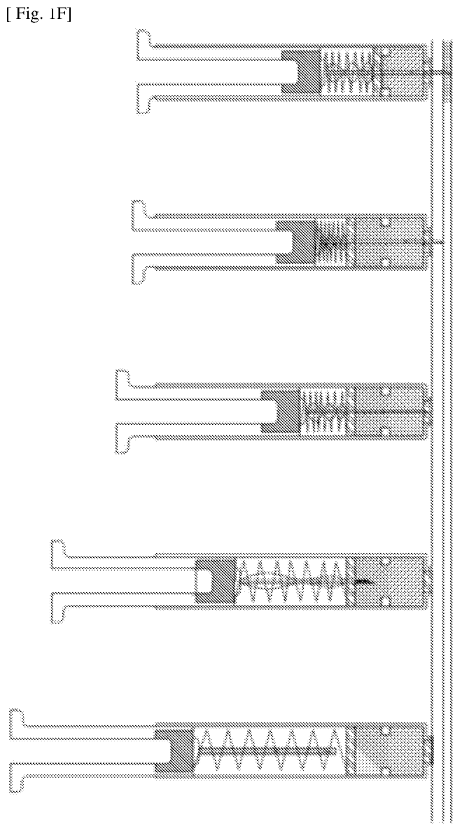

Reference numerals and exemplary corresponding structures are provided below for illustration only, for instance, with reference to FIGS. 1A-1E through FIGS. 11A-11B, and should not be considered limiting: 1—syringe barrel; 1a—axial stopper; 1b—circular contacting element; 2—pressing element; 2c—central guiding groove; 3—floating seal; 3a—angled guiding groove; 4—elastic sheath; 5—spring; 6—hollow puncture needle; 6a—needle distal opening; 6b—needle body opening; 6c—angled guiding needle hole; 7—flowable composition lumen; 8—distal seal; 9—one-way valve; 10—needle hole plug; 11—catheter; 12—auxiliary guiding needle; 13—sclera; 14—suprachoroidal space (SCS).

FIG. 13 shows schematic diagrams of various elements and features of an exemplary medical puncturing device. For instance, the device can comprise a hollow housing 22 engaging a proximal control knob 17. A pressing/push shaft 2 slidably passes through the control knob and engages a guide tube 16 inside the housing. The pressing/push shaft 2 is configured to provide a distally directed force on a compression spring 5, which in turn serves as a force element configured to provide a distally directed force on a piston rod 15. A beveled needle 6, is attached and fixed to a needle base or seat fixed to the pressing/push shaft. The distal end of the needle 6 can reside within a lumen of the piston rod 15 and move distally when a force is applied to move the pressing/push shaft distally. The distal end of the needle can be advanced to pass through a seal 3 at the distal end of the piston rod 15 into a lumen formed by a syringe barrel of a syringe 1 and a distal seal 8. A gland 23 may engage both the syringe 1 and the distal seal 8 to facilitate a sealing engagement. The distal seal 8 can interface a tissue, and the needle 6 can be advanced to pass through the distal seal 8 to penetrate the tissue. The needle 6 may comprise a needle distal opening and a needle body opening, similar to 6a and 6b respectively, as shown and used as described in FIGS. 1A-1E through FIGS. 11A-11B.

In some embodiments, a device disclosed herein comprises a stopper, e.g., limiter 18 in FIG. 13. In some embodiments, the stopper can be used to limit the maximal length of an axial movement of the pressing shaft, e.g., in order to achieve precise injection. In some embodiments, the stopper can be used to limit a rotation and/or a radial movement of the pressing shaft, e.g., in order to prevent or minimize deviation of the pressing shaft (and the needle base and syringe needle coupled thereto) from a central axis of the assembled device. In some embodiments, the stopper can engage the guide tube. In some embodiments, the stopper can engage the fixedly or removably engage the proximal end of the guide tube. In some embodiments, the guide tube can be used to guide the movement of the pressing shaft and the piston rod, e.g., through corresponding structures on the components, in order to achieve precision of the axial movement of the pressing shaft and the piston rod, as well as precision of the syringe needle movement. In some embodiments, the device through a combination of features (e.g., the stopper and the guide tube) prevents or minimizes the rotation and/ or deviation (e.g., from a central axis) of the pressing shaft, the piston rod, the needle base or seat, and/or the syringe needle, both during transportation and storage of the assemble device and during the use of the device for medical penetration.

In some embodiments, a device disclosed herein comprises a ruler, e.g., ruler 19 in FIG. 13. In some embodiments, the ruler can be used to measure or otherwise determine a distance between a penetration site (e.g., a site to be penetrated by the syringe needle) and the corneal limbus, which is the border between the cornea and the sclera. In some embodiments, the distal end of the ruler can be configured to contact a portion of the eye at the injection site. In some embodiments, the protrusion of the ruler can be configured to leave a marker on a portion of the eye, for example, at the injection site. For example, the marker can indicate the injection site. For example, the marker may appear as parallel marker lines on the conjunctiva of the eye, indicating to the user that injection should be performed in the area between the parallel marker lines. In some embodiments, the ruler may be removably coupled to the delivery device, for example, at the distal end (e.g., coupled to the distal seal 8 in FIG. 13 or coupled to the contacting element 1b in FIG. 1A) of the delivery device. In such embodiments, the ruler may be removed from the delivery device after marking the injection site on the target tissue.

FIGS. 14A-14F show schematic diagrams of the different stages of operating an exemplary medical puncturing device.

Reference numerals and exemplary corresponding structures are provided below for illustration only, for instance, with reference to FIG. 14 through FIGS. 14A-14F, and should not be considered limiting: 1—syringe having a syringe barrel forming a lumen; 2—pressing element (e.g., pressing shaft) ; 3—floating seal (e.g., plunger seal); 5—elastic element such as a spring; 6—hollow puncture needle (needle distal opening and needle body opening not shown) ; 8—distal seal; 15—piston rod (e.g., push rod); 16—guide tube; 17—control knob; 18—limiter; 19—ruler; 20—adapter; 21—handle; 22—housing; 23—gland.

FIGS. 15A-15H show schematic diagrams of exemplary contact members, second elastic elements, and connectors, as parts of adapters. The distal seal can function as a contact member that contacts a tissue surface at the beginning of a penetration operation. The distal seal can be rigid or substantially not compressible, or can comprise a combination of rigid, semi-rigid, soft, and/or elastic materials. The needle base can be connected to the distal seal via one or more springs or other elastic materials configured to control the pressure during penetration and/or injection, thereby enhancing safety. There can be provided a sandwich structure (e.g., as shown in FIG. 15D and FIG. 15E) between the needle base and the tissue surface, the sandwich structure comprising a rigid or substantially not compressible material (e.g., in the distal seal) sandwiched by a first soft and/or elastic material on the distal side (e.g., in the distal seal) and a second soft and/or elastic material on the proximal side (e.g., a spring or elastic sheath surrounding the needle). The first and second soft and/ or elastic materials can be the same or different. The distal seal, spring, and/or elastic sheath surrounding the needle cam help reduce the risk of axial movement (e.g., axial deviation) and/or slippage of the needle during the penetration. A soft and/or elastic material used at the distal end can improve the tight or sealing engagement between the contact member (e.g., the distal seal) and the tissue, especially when the tissue surface is uneven and/or when the needle is not perpendicular to the tissue surface, e.g., as shown in FIG. 15G and FIG. 15H.

Reference numerals and exemplary corresponding structures are provided below for illustration only, for instance, with reference to FIG. 15 through FIGS. 15A-15H, and should not be considered limiting: 1—syringe barrel; 6—hollow puncture needle (needle distal opening not shown) ; 25—contact member; 25a—first part of the contact member; 25b—second part of the contact member; 26—second elastic element; 27—connector.

FIGS. 16A-16B show schematic diagrams of exemplary pressing element as a part of adapters (e.g., for a suitable syringe such as a syringe with or without a floating seal) to apply pressure to the plunger of the syringe (e.g., after liquid is drawn into the syringe but before penetration of needle into a tissue).

Reference numerals and exemplary corresponding structures are provided below for illustration only, for instance, with reference to FIG. 16 through FIGS. 16A-16B, and should not be considered limiting: 1—syringe barrel; 2—push shaft; 30—pressing element; 31—elastic element; 32—stopper or locking element.

FIGS. 17A-17B show schematic diagrams of different stages of operating a syringe installed with the adapters described herein.

Reference numerals and exemplary corresponding structures are provided below for illustration only, for instance, with reference to FIG. 17 through FIGS. 17A-17B, and should not be considered limiting: 1—syringe barrel; 2—push shaft; 3—floating seal; 25—contact member; A—a denser tissue; B—a less dense tissue.

In FIG. 17A, the initial state of a syringe (e.g., a regular syringe) is shown in step a, and after drawing a liquid the state of the syringe is shown in step b. At this time, if the needle of the syringe is inserted into a contact member (e.g., a distal seal), which seals the opening of the needle, the liquid inside the syringe can be kept in a sealed space, as shown in step c. Tissue A can be a tissue higher in tissue resistance (e.g., a higher density tissue), and Tissue B is a tissue lower in tissue resistance (e.g., a lower density tissue) or a potential or apparent void, cavity, or vessel. In order to inject the liquid into Tissue B, the distal seal can be contacted with the surface of Tissue A by applying a pressure on the Tissue A surface in step d. As the needle advances, the needle tip first advances inside the distal seal (as shown in step e), and then arrives at the interface between the distal seal and Tissue A (as shown in step f). Then, the needle tip enters Tissue A (as shown in step g), following by the needle tip advancing to the interface between Tissue A and Tissue B (as shown in step h). If a suitable force is applied to the plunger of the syringe during steps e-h, when the needle tip is at a position shown in steps e-g, the fluid cannot be discharged inside the distal seal or Tissue A, since the pressure resistance in the distal seal and Tissue A is greater. When the needle tip reaches the Tissue A and Tissue B interface in step h, since the liquid pressure at the needle tip is greater than the pressure inside Tissue B (e.g., a potential or apparent void, cavity, or vessel), the liquid can be automatically discharged into Tissue B, and the plunger seal moves distally towards the tissues, indicating to the operator that the needle tip is at the Tissue A and Tissue B interface. If needle advancement is stopped but a pressure is still applied to the plunger, as long as the liquid pressure at the needle tip is still greater than the pressure inside Tissue B, then the liquid can continue to be discharged into Tissue B (in step i) until the pressures reach an equilibrium or the plunger seal has reached a limit (e.g., stopped by a stopper which can be provided in the syringe), thereby achieving precise placement of the needle tip at the Tissue A and Tissue B interface and automatic injection. The distal seal (as an adapter for the syringe) that is applied to the needle tip in step c can help secure the liquid inside a sealed lumen, and this time another adapter (e.g., a pressing element for applying pressure) for the syringe can be used to apply a suitable pressure to the liquid inside the syringe through the plunger, and during the operation the operator only needs to pay attention to the force applied to the plunger or the syringe barrel to advance the needle, the speed of needle advancement, and whether the plunger seal suddenly moves downward (distally towards the tissue), and the operator does not need to at the same time monitor and/or adjust the pressure applied to the liquid inside the syringe (to make sure it is greater than the pressure inside Tissue B), thereby reducing the number of various aspects that the operator has to pay attention to during the tissue penetration and improving the safety of the tissue penetration (e.g., to avoid overshooting and damaging deeper tissue). By applying the distal seal to the needle tip, the actual distance of needle advancement during the penetration is greater than the distance of needle advancement inside Tissue A. If Tissue A is a relatively thin tissue, then increasing the actual distance of needle advancement during the penetration can provide better control of the injection and decrease the difficulty of operation. In addition, the distal seal can help reduce the maximum distance of needle advancement (the maximum distance of the needle that goes beyond the distal seal) during the penetration, which if properly controlled can help reduce the risk of overshooting. Moreover, the distal seal in step c can also increase the rigidity of the needle, and reduce the risk of getting bent, axial movement (e.g., axial deviation) and/or slippage of the needle during the penetration.

In FIG. 17B, the initial state of a syringe (e.g., with two seals, one proximal plunger seal, the other being a distal floating seal) is shown in step a, and after drawing a liquid the state of the syringe is shown in step b. At this time, if the needle of the syringe is inserted into a contact member (e.g., a distal seal), which seals the opening of the needle, the liquid inside the syringe can be kept in a sealed space, as shown in step c. Tissue A can be a tissue higher in tissue resistance (e.g., a higher density tissue), and Tissue B is a tissue lower in tissue resistance (e.g., a lower density tissue) or a potential or apparent void, cavity, or vessel. In order to inject the liquid into Tissue B, the distal seal can be contacted with the surface of Tissue A by applying a pressure on the Tissue A surface in step d. As the needle advances by pushing the plunger, the needle tip first advances inside the distal seal (as shown in step e), and then arrives at the interface between the distal seal and Tissue A (as shown in step f). Then, the needle tip enters Tissue A (as shown in step g), following by the needle tip advancing to the interface between Tissue A and Tissue B (as shown in step h). When the needle tip is at a position shown in steps e-g, the fluid cannot be discharged inside the distal seal or Tissue A, since the pressure resistance in the distal seal and Tissue A is greater. When the needle tip reaches the Tissue A and Tissue B interface in step h, if at this time the liquid pressure at the needle tip is greater than the pressure inside Tissue B (e.g., a potential or apparent void, cavity, or vessel), the liquid can be discharged into Tissue B. At this time, if the plunger continues to be pushed distally and the pressure is within a suitable range, the liquid pressure at the needle tip can be maintained to be greater than the tissue pressure inside Tissue B (e.g., a potential or apparent void, cavity, or vessel), while at the same time the liquid pressure on the floating seal (which engages the needle base and is attached to the needle) is less than the friction force, such that the liquid can continue to be discharged into Tissue B (in step i) while the needle tip is not further advanced distally, until the pressures reach an equilibrium or the plunger seal has reached a limit (e.g., stopped by a stopper which can be provided in the syringe). The friction force can be the sum of the static friction forces between the needle and the distal seal and between the needle and Tissue A. By applying the distal seal to the needle tip, the actual distance of needle advancement during the penetration is greater than the distance of needle advancement inside Tissue A. If Tissue A is a relatively thin tissue, then increasing the actual distance of needle advancement during the penetration can provide better control of the injection and decrease the difficulty of operation. When the liquid is discharged into Tissue B (in step i), in order to avoid the needle further advancing distally into Tissue B, the liquid pressure on the floating seal is less than the sum of the static friction forces between the needle and the distal seal and between the needle and Tissue A. Therefore, while the liquid is discharged into Tissue B (in step i), compared to operations without using the distal seal (where the static friction force between the needle and the distal seal is 0 since there is no distal seal), by using a distal seal the penetration operation can tolerate greater forces applied to the plunger (which forces are transmitted to floating seal via the liquid which is largely not compressible) without risking overshooting of the needle tip, thereby increasing safety of the penetration operation. In addition, the distal seal can help reduce the maximum distance of needle advancement (the maximum distance of the needle that goes beyond the distal seal) during the penetration, which if properly controlled can help reduce the risk of overshooting. Moreover, the distal seal in step c can also increase the rigidity of the needle, and reduce the risk of getting bent, axial movement (e.g., axial deviation) and/or slippage of the needle during the penetration.

FIGS. 18A-18B show schematic diagrams of exemplary methods and compositions for drainage through the SCS. In the exemplary ab externo method shown in FIG. 18A, an viscoelastic agent is injected between the sclera and the choroid, forming the SCS, followed by implanting a permanent or semi-permanent structure (e.g., a stent) to keep the SCS in an expanded state for a prolonged period of time. The implant can form a ring or a partial ring on a plane that is parallel to the equator of the eye ball, as shown in FIG. 18B.

### DETAILED DESCRIPTION

Below is a detailed description of some embodiments of the present disclosure. It should be understood that the specific implementations described herein are meant to illustrate and explain the embodiments of the present disclosure, and should not be considered limiting.

It should be noted that, when not in conflict, the embodiments of the present disclosure and the features of the embodiments may be combined in any suitable manner.

In some embodiments, the positional descriptions of “front,” “back,” “forward,” “backward,” “distal,” and “proximal,” etc. are based on the perspective of an operator of the medical puncturing device or medical apparatus assembly. That is, when the operator is using the medical puncturing device or medical apparatus assembly, the direction pointing away and relatively far from the operator is the forward direction, and the direction pointing toward and relatively close to the operator is the backward direction.

As used herein, the words “proximal” and “distal” refer to the direction closer to and away from, respectively, an operator (e.g., surgeon, physician, nurse, technician, etc.) who would insert the medical device into the patient, with the tip-end (distal end) of the device inserted inside a patient's body first. Thus, for example, the end of a needle (e.g., microneedle) described herein first inserted inside the patient's body would be the distal end, while the opposite end of the needle (e.g., the end of the medical device being manipulated by the operator) would be the proximal end of the needle.

As used herein, the singular forms “a,” “an,” and “the” include plural referents unless the context clearly dictates otherwise. For example, “a” or “an” means “at least one” or “one or more.” Likewise, the term “a member” is intended to mean a single member or a combination of members, “a material” is intended to mean one or more materials, or a combination thereof.

The term “about” or “approximately” as used herein refers to the usual error range for the respective value readily known to the skilled person in this technical field. Reference to “about” a value or parameter herein includes (and describes) embodiments that are directed to that value or parameter per se. For example, “about” can mean within 1 or more than 1 standard deviation, per the practice in the relevant field. Alternatively, “about” can mean a range of up to 20%, up to 10%, up to 5%, or up to 1% of a given value.

As used herein, the terms “pre-filled”, “pre-loaded”, and “pre-packaged” refer to a state of injection device or systems where a composition (e.g., drugs or medicaments) has been filled into the injection device or systems prior to the use of these injection device or systems. In some embodiments, “pre-filled”, “pre-loaded”, and “pre-packaged” injection device or systems encompasses injection device or systems that are filled with a medicament and stored in this pre-filled form for a period of time before administration of the medicament to a subject. In some embodiments, “pre-filled”, “pre-loaded”, and “pre-packaged” injection device or systems encompasses injection device or systems that are filled with all the medicament (s) to be administered and stored in this pre-filled form for a period of time before administration of the medicament(s) to a subject. In some embodiments, “pre-filled”, “pre-loaded”, and “pre-packaged” injection device or systems encompasses injection device or systems that are filled with part or certain component of the medicament to be administered and stored in this pre-filled form for a period of time before administration of the medicament to a subject, and the remaining part and components are filled immediately prior to the administration.

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the terms “puncture member”, and “puncturing member” are used interchangeably to refer to an article configured to pierce tissue layers and deliver a substance to a target tissue layer, for example, a needle or a microneedle.

As used herein, the terms “medicament container”, and “medicament containment chamber” are used interchangeably to refer to an article (e.g., a syringe) configured to contain a volume of a substance, for example, a medicament or drug.

As used herein, the term “syringe” can include any type of commonly used syringes, which comprise a needle with a proximal end and a distal end, a syringe barrel with a distal end and an open proximal end, and a pushing shaft with a distal end and a proximal end, wherein the proximal end of the needle is connected to the distal end of the syringe barrel, wherein a fluid lumen is formed between the distal end of the pushing shaft and the distal end of the syringe barrel, and wherein a fluid communication can be built from the fluid lumen to the proximal end of the needle, and further to the distal end of the needle. The term “syringe” can cover all common syringes regardless of size or volume. For example, an 1 μL syringe is encompassed within the “syringe” described herein, and an 1 L syringe is also encompassed within the “syringe” described herein. In some embodiments, a syringe may not comprise any valves. In some embodiments, a syringe may not comprise any springs. In some embodiments, a syringe may not comprise not comprise any floating seals.

In some embodiments, the terms “connecting” and “engaging” are intended to encompass both the situation wherein the two components being connected or engaged are tied together and cannot move apart, and the situation wherein the two components touch each other while not being tied together. For example, an elastic element connected to a needle base could mean the elastic element is tied to the needle base and always stays with the needle base, and could also mean the elastic element can touch the needle base when the needle is moved to a certain position, but may not touch the needle base at certain time points.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Direct delivery of drugs and/or implants into eyes has been a major pathway to treat a wide variety of eye diseases. Among the drug delivery methods, intravitreal injection has been a mainstream method, and constitute an effective approach to achieving high intraocular levels of antibiotic, antiviral, antifungal, steroidal, and anti-VEGF drugs (Peyman G. A. et al. Retina. 2009, 29 (7), 875-912). Intravitreal injections have a good safety profile, but they can cause ocular complications, including cataract formation, glaucoma, choroidal hemorrhage, endophthalmitis, vitreous hemorrhage, and retinal detachment, and the injected drug can be absorbed and induce systemic adverse effects (Prasad A. G. et. al., Compr Ophthalmol Update. 2007, 8 (5), 259-269). Some drugs or gene therapy vectors may penetrate the posterior vitreous membrane and the inner layer of the retina to reach the outer layer of the retina or the retinal pigment epithelium layer, thereby have low efficiency. In addition, because the vitreous cavity is a semi-open cavity, the drugs injected into the vitreous cavity are likely to flow out of the eyeball with the circulation of aqueous humor, which affects the local concentration and pharmacokinetics of the drug, and may also cause side effects such as increased intraocular pressure and cataracts. Another common method for drug delivery, topical administration of drugs, generally achieves low concentration in the vitreous, and is not commonly used for the treatment of eye diseases, especially diseases that are not on surface of eye (Abdelkader H. et. al., Curr Drug Deliv. 2012, 9 (4), 421-430). Systemic treatment is also limited, as the blood-retina-barrier limits the ability of the drugs to reach the eye, and high doses may cause systemic adverse effects (Rai Udo J. et. al., Drug Discov Today. 2015, 20 (4), 491-495). Periocular (subconjunctival, sub-Tenon, or retrobulbar) injections can bypass the blood-retina-barrier without intraocular penetration. However, the injected drug still has to traverse the sclera, which is less permeable to larger molecules, and may not achieve therapeutic drug levels at the level of the retina (Geroski D. H. et. al., Adv Drug Deliv Rev. 2001, 52 (1), 37-48).

In contrast, suprachoroidal injection can achieve a higher drug concentration in the choroid/ciliary body, retinal pigment epithelium and/or outer layer of the retina, and the vitreous drug concentration is lower. Animal studies have shown that the SCS can accommodate up to 1 mL of fluid, which rapidly diffused into the posterior segment (Seiler G. S. et. al., Invest Ophthalmol Vis Sci. 2011, 52 (8), 5730-5736). This volume is much larger than what is required for achieving therapeutic levels for clinically relevant drugs. Injections of 10-50 μL into the SCS have been demonstrated to be well tolerated with a low risk of ocular complications (Gu B. et. al., Invest Ophthalmol Vis Sci. 2015, 56 (6), 3623-3634). Therefore, SCS is an attractive route for drug delivery, as it may allow for larger volumes of drugs and longer duration of action to be achieved with a safer procedure. In addition, drug delivery through the SCS bypasses the internal limiting membrane barrier and outer blood-retina-barrier, and may be a preferred route for drug delivery targeting the retinal pigment epithelium.

However, precise drug delivery into the suprachoroidal space (SCS) is challenging. In some instances, targeted injection of a therapeutic agent is desirable. In such instances, however, the relatively small anatomic structures of the eye often result in significant challenges to placing a needle at a target location using known devices and methods, especially as they pertain to placing the distal end of the needle at the desired depth within the eye. Many known methods of direct injection of a drug into the eye include inserting a needle or a cannula at an acute angle relative to a surface of the eye, which can make controlling the depth of insertion challenging. For example, some such methods include controlling the angular orientation of the needle such that the injected substance exits the needle at a particular location. Moreover, some known methods of injecting substances into ocular tissue include using complicated visualization system or sensors to control the placement of the needle or cannula.

Such shortcomings in known systems and methods are exacerbated because the size and thickness of various layers included in the eye can vary substantially from one person to another. For example, the thickness of the conjunctiva and the sclera can be substantially different and their true value cannot easily be predetermined via standard techniques. Furthermore, the thickness of these layers can also be different in different portions of the eye and at different times of the day in the same eye and location. Therefore, using known systems and methods it can be challenging to determine and/or adjust the length of the needle for puncturing the eye, such that a tip of the needle is at the desired depth, for example, the SCS.

In some cases, such as choroidal melanoma, precise targeted injection of therapeutic agents into the suprachoroidal space may improve efficacy and reduce side effects. However, for the eye, its structure is small, and it is quite difficult to use existing devices or methods to realize the penetration, expansion, injection, or catheter placement of the suprachoroidal space, especially for medical devices such as catheters, especially when the device needs to be placed in a specific location in the suprachoroidal space.

Some puncture methods for the suprachoroidal space are to make the length of the exposed puncture needle equal to the thickness of the sclera. After the puncture needle is completely inserted into the sclera, fluid is injected to achieve the suprachoroidal injection. The technical disadvantage of this puncture method is that the exposed length of the reserved puncture needle may not be exactly the same as the thickness of the sclera. In practical applications, the difference in the thickness of the sclera between different people, between eyes, and between different parts of the same eye will further amplify the above technical defects. Too short a needle might not penetrate the sclera, and too long a needle can traverse beyond the SCS and damage the retina of the eye. A convenient way to detect the position of the needle tip within the eye is needed.

Because of the sensitivities associated with intraocular injection (e.g., the sensitivity of the tissue, the potential impact on intraocular pressure and the like), many known systems involve manual injection. More particularly, many known devices and methods include the user manually applying a force (e.g., via pushing a plunger with their thumb or fingers) to expel a fluid (e.g., a drug) into the eye. Because of the small needle size and/or the characteristics of the injected drug, some such devices and methods involve the use of force levels higher than that which users are comfortable with applying, and in certain situations a user may not properly deliver the medicament using known systems and methods.

Moreover, injection into different target layers of the eye can cause variability in the amount of the force required for insertion of the needle and/or injection of the medicament. Different layers of the eye can have different densities. For example, the sclera generally has a higher density than the conjunctiva or the SCS. Differences in the density of the target region or layer can produce different backpressure against the needle exit, e.g., the tip of the needle from which the fluid emerges. Thus, injection into a relatively dense ocular material such as sclera requires more motive pressure to expel the medicament from the needle than is required when injecting a medicament into the SCS. Furthermore, the injection force to expel the medicament also depends on the density and viscosity of the liquid medicament, length of the needle, and diameter of the needle. To inject certain medicaments into the eye via desired needles (e.g., 27 gauge, 30 gauge, or even smaller) can require force which may be difficult to estimate and/or control in order to achieve proper injection without risking damage to eye tissues of a particular subject.

Thus, a need exists for improved devices and methods, which can assist in determining if the needle is at the correct depth, can facilitate injection of the medicament into tissues such as an ocular tissue, and/or can facilitate the implant of certain structures in tissues such as an ocular tissue.

Further challenges involved in the precise delivery of drugs and/or implants into SCS include the high requirement for sterility for eye injections and the requirement for precise dosage amount. For example, it has been demonstrated that the risk of endophthalmitis after intravitreal injections of anti-vascular endothelial growth factor (anti-VEGF) agents is significantly affected by the sterility of the drug composition and the drug loading environment (VanderBeek B. L., JAMA Ophthalmol., 2019, 137 (4), 343-344). Compared to injection device or systems that are pre-loaded or pre-filled with drug agents, the office-prepared injections show significantly higher rates of endophthalmitis. By eliminating the in-office transferring of the medication, a common source of contamination would be removed and contamination-related adverse effects can be significantly reduced. In the meanwhile, precise control of the dosage amount and volume is also crucial, especially for small spaces and thin tissues in eyes. Therefore, there remains a great need for injection device or systems that not only enables precise injection into SCS, but also improves the sterility, achieves better control over dosage amount, and minimize inconvenience caused to medical personnel.

In order to achieve one or more of the purposes mentioned above, in some aspects, the present disclosure provides a pre-filled medical puncturing device for injecting a drug composition, comprising:
a syringe barrel comprising a proximal end and a distal end;
a floating seal in the syringe barrel;
a needle base proximal to the floating seal, wherein the floating seal and the needle base elastically engage each other;
a needle for insertion into the eye, the needle comprising:
(i) a needle proximal end engaging the needle base;
(ii) a needle distal end;
(iii) a needle distal opening;
(iv) a needle body opening between the needle proximal end and the needle distal end, wherein the needle body opening is proximal to the needle distal opening; and
(v) a needle body passageway connecting the needle distal opening and the needle body opening,
wherein the needle base is configured to advance the needle distally toward and/or through the floating seal; and
wherein a drug composition is contained in a lumen formed by the floating seal and the distal end of the syringe barrel.

In other aspects, provided herein is a set of adapters that can be combined with a syringe to significantly improve the precision of inject depth of the syringe, facilitate injection of the medicament into tissues such as an ocular tissue, and/or can facilitate the implant of certain structures in tissues such as an ocular tissues. In some aspects, the adapters described herein can improve the injection precision and safety of a syringe, once installed onto the syringe. In some embodiments, the adapters described herein can improve the injection precision and safety of other syringes, for example, the syringe disclosed in US 2020/0069883, once installed onto the syringe. In some aspects, the set of adapters comprises:
a contact member extending from a proximal end to a distal end;
and a pressing unit comprising a first elastic element;
wherein the contact member can be assembled to the needle distal end of a syringe such that the distal end of the contact member is distal to the needle distal end opening, and the distal end of the contact member can directly contact with surface tissues at a target injection site; and
wherein the pressing unit can be assembled to a syringe barrel and push shaft such that the pressing unit elastically engages the push shaft and/or the syringe barrel via the first elastic element.

In yet other aspects, provided herein is a method of improving the injection precision and safety of a syringe, comprising:
(1) providing a syringe comprising: a syringe barrel extending from a proximal end to a distal end and forming a lumen extending from the proximal end to the distal end; a push shaft extending from a proximal end to a distal end and forming a seal between the distal end of the push shaft and the syringe barrel; and a needle extending from a proximal end to a distal end comprising an end opening for the fluid to flow from the lumen and through the distal end of the syringe barrel via a needle base;
(2) providing a set of adapters comprising: a contact member extending from a proximal end to a distal end, and a pressing unit comprising a first elastic element;
(3) installing the contact member to the distal end of the needle of the syringe;
(4) installing the pressing unit to the syringe,
wherein the distal end of the contact member is distal to the needle distal end opening, and the distal end of the contact member can directly contact with surface tissues at a target injection site; and
wherein the pressing unit elastically engages the push shaft and/or the syringe barrel via the first elastic element.

In some embodiments, following the injection into the SCS, one or more structures such as a permanent or semi-permanent stent can be implanted into the SCS to maintain it for a prolonged period of time, for instance, at least 4, 6, 8, 10, 12, 24, or 36 months or even longer. In some embodiments, a method disclosed herein comprises: (a) inserting a needle into the eye at an injection site for injection into a suprachoroidal space (SCS) in the eye; (b) delivering a composition (e.g., a viscoelastic composition) through the needle to form the SCS; and (c) through the injection site or an expanded insertion site (e.g., formed by expanding the injection site, for instance, by surgery), positioning a stent (e.g., a micro stent) in the SCS, thereby placing the stent in the eye to sustain the SCS in an expanded state and facilitate drainage of aqueous humor.

Some embodiments of the present disclosure will be described with reference to the several views of the accompanying drawings.

### II. Injection Devices or Systems

Integrated devices comprising drug formulations (e.g., pre-filled syringes) are disclosed. In some aspects, provided herein is a pre-filled injection device or system comprising an injection device to assist in the insertion of a puncture member, for example, a needle or microneedle into the eye, and/or assist in injecting a medicament pre-filled in the device into a target ocular tissue. In some embodiments, described herein is a pre-filled injection device or system comprising an injection device for controlling the insertion depth of a puncture member, such as, for example, a microneedle, into the eye to deliver a therapeutic agent pre-filled in the device to, for example, a posterior region of the eye (e.g., via the suprachoroidal space). In some embodiments, described herein is a pre-filled injection device or system comprising an injection device for introducing an implant pre-filled in the device into a tissue, such as an apparent or potential tissue void, cavity, or vessel.

In some embodiments, provided herein is a pre-filled injection device or system comprising a syringe barrel comprising a proximal end and a distal end; a floating seal in the syringe barrel; a needle base proximal to the floating seal (e.g., the needle base is closer to an operator while the floating seal is closer to a subject), and the floating seal and the needle base are configured to elastically engage each other. In some embodiments, the pre-filled injection device or system further comprises a needle comprising a needle proximal end and a needle distal end, and the needle proximal end engages the needle base. In any of the embodiments herein, the needle proximal end can be fixed to the needle base or releasably attached to (e.g., inserted in) the needle base. In any of the embodiments herein, the needle can comprise: (i) a needle distal opening, (ii) a needle body opening between the needle proximal end and the needle distal end, and (iii) a needle body passageway connecting the needle distal opening and the needle body opening. In any of the embodiments herein, the needle body opening can be proximal to the needle distal opening. In any of the embodiments herein, the needle base can be configured to advance the needle distally toward the floating seal (e.g., when the needle distal end is proximal to the floating seal), through the floating seal (e.g., when the needle distal end has entered or pierced into the floating seal), and/ or through the distal end of the syringe barrel.

In any of the embodiments herein, a proximal lumen and a distal lumen can be provided in the syringe barrel on different sides of the floating seal. In some embodiments, the distal lumen comprises a pre-filled flowable composition (e.g., a medicament, a drug, and/or a pharmaceutically acceptable carrier or excipient such as a saline), while the proximal lumen does not contain a non-gas flowable composition. The proximal lumen may be pre-filled with a gas, such as a sterilized air, and/or capable of communicating with the outside environment such as the atmosphere when the needle is advanced in and/or through the syringe barrel.

In some embodiments, the needles included in the embodiments described herein comprise a bevel, which allows for ease of penetration into a tissue such as the sclera and/or suprachoroidal space with minimal collateral damage. In some embodiments, the needles disclosed herein can define a narrow lumen (e.g., gauge size greater than or equal to 30 gauge, 32 gauge, 34 gauge, 36 gauge, etc.) to allow for suprachoroidal drug delivery while minimizing the diameter of the needle track caused by the insertion of the needle. In some embodiments, the lumen and bevel aspect ratio of the needles described herein are the same or different from standard 27 gauge and 30 gauge needles commonly used for intraocular injection.

In some embodiments, a pre-filled injection device or system disclosed herein comprises an injection device which comprises or is configured to be coupled to a pre-filled medicament container containing a medicament, such as a gel or the like. The medicament container can be formed at least in part by the syringe barrel.

In some embodiments, a needle is coupled to a distal end of a pre-filled medicament container (e.g., the needle is at the distal end of a syringe), for example, as described in U.S. Pat. Nos. 9,180,047, 9,539,139, 9,572,800, 9,636,253, 9,636,332, 9,770,361, 9,937,075, 10,555,833, and 10,517,756, which are incorporated herein by reference for all purposes. In other embodiments, the present disclosure utilizes a needle that is coupled to an actuation member inside a syringe barrel. In some embodiments, a needle disclosed herein is at least partially inside the syringe barrel. In some embodiments, prior to use, the needle neither is exposed at the distal end of the syringe barrel nor directly engages the distal end of the syringe barrel.

In some embodiments, a pre-filled injection device or system disclosed herein comprises an injection device which comprises an energy storage member (e.g., one or more springs) configured to engage the needle base and the floating seal. In some embodiments, a distal end portion of the energy storage member is configured to be disposed within the syringe barrel and directly or indirectly engage the floating seal. In some embodiments, the energy storage member is configured to produce a force on a proximal end portion of the floating seal. In some embodiments, the force is sufficient to move the floating seal within the syringe barrel to convey at least a portion of a substance from the medicament container (e.g., a flowable composition lumen) via the needle when a distal tip of the needle is disposed within an apparent or potential tissue void, cavity, or vessel. Furthermore, the force is insufficient to move the floating seal within the syringe barrel when the distal tip of the needle is disposed within a tissue adjacent to (e.g., above or below) the apparent or potential tissue void, cavity, or vessel. In some embodiments, the apparent or potential tissue void, cavity, or vessel has a first density and the adjacent tissue has a second density, higher than the first density. In some embodiments, the apparent or potential tissue void, cavity, or vessel produces a first backpressure and the adjacent tissue produces a second backpressure, higher than the first backpressure.

In some embodiments, a needle is coupled to a floating seal. In other embodiments, the present disclosure utilizes a needle whose proximal end is coupled to an actuation member inside a syringe barrel, where the actuation member is separately provided and is proximal to the floating seal. In some embodiments, the proximal end of a need disclosed herein is not coupled to the floating seal. In some embodiments, prior to use, the needle can be distal to the floating seal or can be through the floating seal, but the proximal end of the needle remains distal to the floating seal and is not fixedly attached to the floating seal.

In some embodiments, a pre-filled injection device or system disclosed herein comprises a pre-filled medicament container (e.g., comprising a liquid) between a proximal seal and a distal seal that each can move within a syringe barrel, for example, as described in U.S. Pat. No. 11,413,397, and US 2020/0069883 which are incorporated herein by reference for all purposes. In some embodiments, provided herein is a system pre-filled with any one or more of the compositions disclosed herein (for example, a drug formulation disclosed in Section III herein) for delivering the composition(s) into an apparent or potential void, cavity, or vessel within a subject, the system comprising: a syringe barrel extending from a first end to a second end and forming a lumen extending from the first end to the second end; a plug arranged within the lumen proximate to the first end and forming a seal between the plug and the syringe barrel against the movement of a fluid (which is pre-filled in the lumen) from the lumen between the plug and the syringe barrel; a floating seal arranged within the lumen proximate to the second end forming a seal between the floating seal and the syringe barrel against the fluid movement from the lumen between the floating seal and the syringe barrel; a hollow needle extending from a proximal end connected to the floating seal to a distal end having an opening for the fluid (e.g., comprising one or more of the compositions disclosed herein) to flow from the lumen, through the floating seal, and through the second end of the syringe barrel via the hollow needle; and wherein the syringe barrel, the plug, and the floating seal include material and dimensions selected based on a threshold flowrate for the fluid arranged within the lumen to: upon a force being applied to the fluid, overcome an opposing force to move the floating seal and the hollow needle from the second end of the syringe barrel and extend the distal end of the hollow needle into a tissue of the subject, and upon the distal end of the hollow needle extending beyond the tissue of the subject and into the void of the subject, succumb to the opposing force to displace the fluid into the void through the opening formed at the distal end of the hollow needle. Any of the adapters disclosed herein (e.g., in Section VII) can be used in conjunction with the system to further improve the injection precision and/or safety.

In some embodiments of the foregoing, a force on the proximal side of the proximal seal is transmitted through the liquid to the distal seal which is attached to a needle. Given liquids are generally incompressible, when an operator uses too much force or applies a force abruptly on the proximal seal (e.g., through a plug coupled to the proximal seal), the force will be transmitted to the needle. In some embodiments, in those devices disclosed in US 2020/0069883, because the liquid providing little compressibility to buffer the impact of the force, the needle may be inserted too deeply or too abruptly, causing damage to the target tissue (e.g., suprachoroidal space) and/or surrounding tissues. Although the positions of the proximal seal and the distal seal may be observed during injection, once a force that may cause overshooting of the needle is applied, it could already to be too late to stop the movement of the needle due to lack of the ability to buffer the impact of the force. In contrast, in some embodiments, the pre-filled injection device or system disclosed herein further comprises a contact member distal to the distal end of the syringe barrel, wherein the contact member is elastically connected to the distal end of the syringe barrel via an elastic element. The contact member can be in direct contact with surface tissues of a target injection site, and the elastic connection can facilitate the operator to apply the right force when inserting the needle and buffer the impact of that force, thereby prevent overshooting of the needle. In some embodiments, the elastic element is in the form a spring, wherein one side of the spring is connected to the contact member and the other side of the spring is connected to the distal seal. In some embodiments, the elastic element is an elastic sleeve or sheath wherein the needle is inside of and surrounded by the elastic sleeve or sheath. In some embodiments, a pre-filled injection device or system disclosed herein could comprise any device disclosed in US 2020/0069883. In some embodiments, a pre-filled injection device or system disclosed herein could comprise any device disclosed in US 2020/0069883, and may further comprise any adapters described herein.

In other embodiments of the present disclosure, a pre-filled injection device or system disclosed herein comprises a pre-filled medicament container (e.g., flowable composition lumen) between a floating seal and the distal end of a syringe barrel (where the distal end does not move relative to the syringe barrel). In some embodiments, the distal end of the syringe barrel comprises a distal seal and the flowable composition lumen is provided between the floating seal and the distal seal. In some embodiments, since the needle base is elastically connected to the floating seal (and therefore the flowable composition), the elastic connection can facilitate the operator to apply the right force and buffer the impact of that force. In addition, an operator can hold the needle base still relative to the syringe barrel and observe the movement of the floating seal in order to assess the depth of needle placement. Once fluidic communication is established between the flowable composition and an apparent or potential tissue void, cavity, or vessel, and the pressure in the flowable composition is greater than that in the apparent or potential tissue void, cavity, or vessel, the floating seal can move as the flowable composition enters the tissue, while the needle and the needle base do not have to move. Thus, precise needle placement and steady injection can be achieved and chances of needle overshooting can be effectively reduced or eliminated.

In some embodiments, a pre-filled injection device or system disclosed herein comprises an injection device, which is provided and/or packaged as an integrated device comprising components engaging each other. In some embodiments, a pre-filled injection device or system disclosed herein comprises an injection device, which does not require an operator to assemble one or more of components prior to use. In some embodiments, a pre-filled injection device or system disclosed herein comprises a pre-filled medicament container (e.g., flowable composition lumen) comprising a flowable composition, such as a medicament in the form of a liquid, a solution, a suspension, a gel, an oil, an ointment, an emulsion, a cream, a foam, a lotion, and/or a paste.

Flowable compositions include liquid (e.g., solution, suspension, or the like) or semi-solid compositions (e.g., gels) that are easy to manipulate and may be injected, shaped and/or molded at or near the target tissue site as it coagulates. “Flowable” includes formulations with a low viscosity or water-like consistency to those with a high viscosity, such as a viscoelastic or a paste-like material. In some embodiments, a method disclosed herein involves injecting a viscoelastic material (e.g., a viscoelastic fluid) into an eye, e.g., between the sclera and the choroid/ciliary body of the eye in order to form a suprachoroidal space containing the viscoelastic material. In some embodiments, a viscoelastic fluid is are a non-Newtonian fluid formed by a viscous component and an elastic one, such as a blend of a solvent and a polymeric material. Examples of viscoelastic materials that can be used herein include sodium hyaluronate, Provisc (1% viscous and transparent material which is a specific fraction of sodium hyaluronate), Viscoat (a dispersive viscoelastic comprising of sodium hyaluronate and chondroitin sulphate), Amvisc (a purified fraction of sodium hyaluronate), Amvisc Plus (a 1.6% sodium hyaluronate product derived from rooster combs), sodium chondroitin sulfate/sodium hyaluronate, or DisCo Visc (4% sodium chondroitin sulfate, 1.65% sodium hyaluronate).

In various embodiments, the flowability of the formulation allows it to conform to irregularities, crevices, cracks, and/or voids in the tissue site. For example, in various embodiments, the formulation may be used to fill one or more voids, expand a tissue void (e.g., an apparent tissue void), and/or create a tissue void from a potential tissue void and optionally expand the created void. In some embodiments, upon contact with an aqueous medium (e.g., body fluid, water, etc.), the flowable composition may harden to form a drug depot that controls drug release.

In some embodiments, the device is pre-filled with a therapeutic agent (e.g., a drug), e.g., as part of the flowable composition. Non-limiting examples of specific drugs and classes of drugs include β-adrenoceptor antagonists (e.g., carteolol, cetamolol, betaxolol, levobunolol, metipranolol, timolol), miotics (e.g., pilocarpine, carbachol, physostigmine), sympathomimetics (e.g., adrenaline, dipivefrine), carbonic anhydrase inhibitors (e.g., acetazolamide, dorzolamide), topoisomerase inhibitors (e.g., topotecan, irinotecan, camptothecin, lamellarin D, etoposide, teniposide, doxorubicin, mitoxantrone, amsacrine), prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), anti-viral compounds (e.g., acyclovir, cidofovir, idoxuridine, interferons), aldose reductase inhibitors, anti-inflammatory and/or anti-allergy compounds (e.g., steroidal compounds such as triamcinolone, betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), artificial tear/dry eye therapies, local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors and treatments of age-related macular degeneration such as pegagtanib sodium, ranibizumab, aflibercept, and bevacizumab.

In one embodiment, the therapeutic agent is an integrin antagonist, a selectin antagonist, an adhesion molecule antagonist (e.g., intercellular adhesion molecule (ICAM)-1, ICAM-2, ICAM-3, platelet endothelial adhesion molecule (PCAM), vascular cell adhesion molecule (VCAM)), a leukocyte adhesion-inducing cytokine or growth factor antagonist (e.g., tumor necrosis factor-aα (TNF-α), interleukin-16 (IL-1β), monocyte chemoattractant protein-1 (MCP-1/CCL2), or a vascular endothelial growth factor (VEGF) ). In some embodiments, a vascular endothelial growth factor (VEGF) inhibitor is administered with one of the microneedles described herein. In some embodiments, two drugs are delivered by the methods described herein. The compounds may be administered in one formulation, or administered serially, in two separate formulations. For example, both a VEGF inhibitor and VEGF are provided. In some embodiments, the VEGF inhibitor is an antibody, for example a humanized monoclonal antibody. In further embodiments, the VEGF antibody is bevacizumab. In another embodiment, the VEGF inhibitor is ranibizumab, aflibercept or pegaptanib. In still other embodiments, the devices and methods described herein can be used to deliver one or more of the following VEGF antagonists: AL8326, 2C3 antibody, AT001 antibody, HyBEV, bevacizumab (Avastin), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, Sutent (sunitinib malate), INDUS815C, R84 antibody, KD019, NM3, allogenic mesenchymal precursor cells combined with an anti-VEGF agent or antibody, MGCD265, MG516, VEGF-Receptor kinase inhibitors, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, Cediranib (Recentin), AV-951 (Tivozanib, KRN-951), Stivarga (regorafenib), Volasertib (BI6727), CEP11981, KH903, Lenvatinib (E7080), terameprocol (EM1421), ranibizumab (Lucentis), Votrient (pazopanib hydrochloride), PF00337210, PRS050, SP01 (curcumin), Carboxyamidotriazole orotate, hydroxychloroquine, linifanib (ABT869, RG3635), Iluvien (fluocinolone acetonide), ALG1001, AGN150998, DARPin MP0112, AMG386, ponatinib (AP24534), AVA101, Vargatef (nintedanib), BMS690514, KH902, golvatinib (E7050), Afinitor (everolimus), Dovitinib lactate (TKI258, CHIR258), ORA101, ORA102, Axitinib (Inlyta, AG013736), Plitidepsin (Aplidin), Lenvatinib mesylate, PTC299, aflibercept (Zaltrap, Eylea), pegaptanib sodium (Macugen, LI900015), Visudyne (verteporfin), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PT1101, TG100948, ICS283, XL647, enzastaurin hydrochloride (LY317615), BC194, quinolines, COT601M06.1, COT604M06.2, Mabion VEGF, SIR-Spheres coupled to anti-VEGF or VEGF-R antibody, Apatinib (YN968D1), and AL3818. In addition, delivery of a VEGF inhibitor or VEGF antagonist using the microneedle devices and methods disclosed herein may be combined with one or more agents listed herein or with other agents known in the art.

In some embodiments, one or more components of the pre-filled injection device or system disclosed herein are configured to be assembled with one another. For example, the system or device may comprise one or more syringe barrels.

In some embodiments, the pre-filled injection device or system may comprise two or more units, such as a first syringe unit comprising: a first syringe barrel; a needle base in the first syringe barrel; and a needle comprising a needle proximal end engaging the needle base and a needle distal end. In some embodiments, the pre-filled injection device or system may comprise a second syringe unit configured to engage a distal end of the first syringe unit, comprising: a second syringe barrel; and a floating seal in the second syringe barrel, and when the first and second syringe units are engaged, the floating seal is configured to elastically engage the needle base. In some embodiments, the pre-filled injection device or system may comprise a third syringe unit configured to engage a distal end of the second syringe unit, comprising a third syringe barrel enclosing a flowable composition, and the needle base can be configured to advance the needle to place the needle proximal end and/or the needle distal end in the flowable composition. In any of the embodiments herein, the pre-filled injection device or system can comprise one or more syringe units, optionally a fourth syringe unit configured to engage a distal end of the third syringe unit.

In some embodiments, the pre-filled injection device or system may comprise a first syringe unit comprising: a first syringe barrel; a needle base and a floating seal in the first syringe barrel elastically engaging each other, the needle base being proximal to the floating seal; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the pre-filled injection device or system may further comprise a second syringe unit configured to engage a distal end of the first syringe unit, comprising a second syringe barrel enclosing a flowable composition, and the needle base can be configured to advance the needle to place the needle proximal end and/or the needle distal end in the flowable composition. In any of the embodiments herein, the device can comprise one or more syringe units, optionally a third syringe unit configured to engage a distal end of the second syringe unit.

In some embodiments, the pre-filled injection device or system may comprise a first syringe unit comprising: a first syringe barrel; a needle base in the first syringe barrel; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the system or device may further comprise a second syringe unit configured to engage a distal end of the first syringe unit, comprising: a second syringe barrel; a floating seal in the second syringe barrel, and when the first and second syringe units are engaged, the floating seal is configured to elastically engage the needle base; and a flowable composition, and the needle base can be configured to advance the needle to place the needle proximal end and/or the needle distal end in the flowable composition. In any of the embodiments herein, the pre-filled injection device or system can comprise one or more syringe units, optionally a third syringe unit configured to engage a distal end of the second syringe unit.

In some embodiments, the present disclosure provides in a the pre-filled injection device or system comprising: a syringe barrel, wherein the syringe barrel comprises a distal closed end and a proximal open end; an actuation unit (e.g., an elastic movement unit) comprising an actuation member (e.g., pressing element) and a floating seal, wherein the floating seal is positioned inside the syringe barrel and can elastically engage with the actuation member (e.g., pressing element) ; a hollow puncture needle attached to the actuation member (e.g., pressing element), wherein the hollow puncture needle comprises a needle distal opening and a needle body opening, and wherein the needle body opening is proximal to the floating seal (the needle distal opening can be proximal to the floating seal, e.g., the entire length of the needle is proximal to the floating seal, or alternatively, the needle can be through the floating seal such that the needle distal opening is distal to the floating seal) ; and a pre-filled flowable composition lumen (e.g., for a fluid or gel), wherein the flowable composition lumen is formed by the syringe barrel distal closed end, a syringe barrel lumen wall (e.g., a portion of the syringe barrel), and the floating seal.

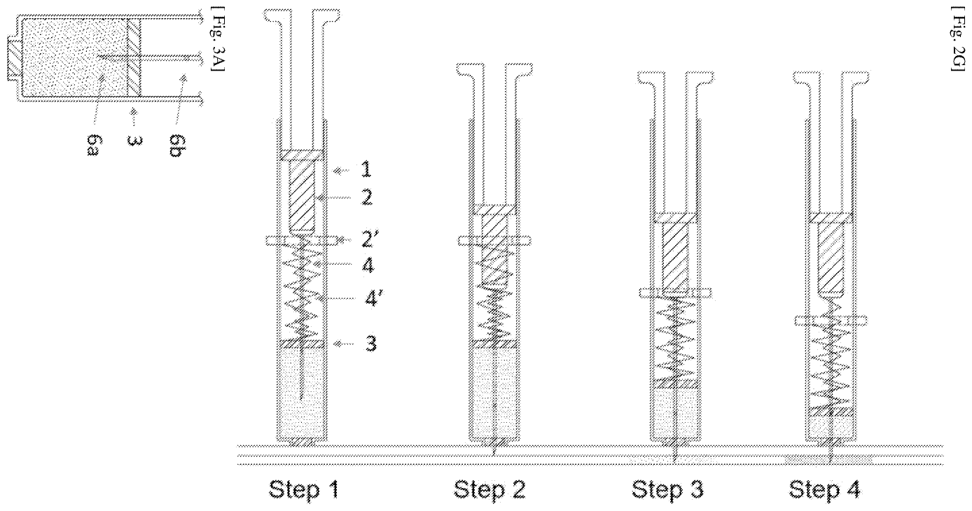

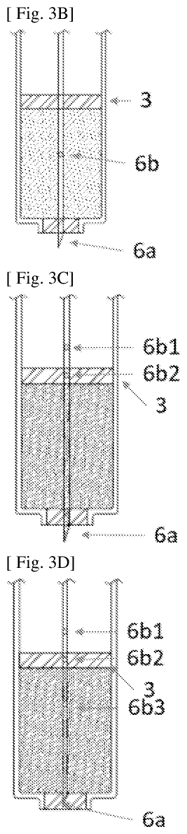

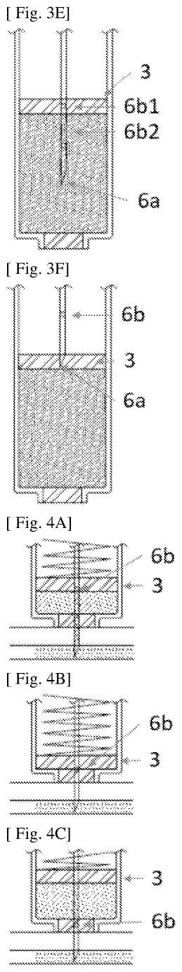

In some embodiments, the pre-filled injection device or system is configured such that the hollow puncture needle can be moved forward by pressing the actuation member (e.g., pressing element). In some embodiments, the hollow puncture needle sequentially pierces the floating seal and the syringe barrel distal closed end, thus connecting the flowable composition lumen, the needle body opening, and the needle distal opening. In some embodiments, the hollow puncture needle is pre-inserted into the floating seal. For example, the needle distal opening can be in the floating seal and blocked by the floating seal, and the needle can be advanced through the flowable composition lumen to pierce the syringe barrel distal closed end. In some embodiments, the hollow puncture needle is pre-inserted through the floating seal. For example, the needle distal opening can be in the flowable composition lumen, while the needle body opening is proximal to the floating seal or in the floating seal (e.g., the needle body opening can be blocked by the floating seal as shown in FIG. 3E), and then the needle can be advanced to pierce the syringe barrel distal closed end. In some embodiments, the hollow puncture needle is pre-inserted through the floating seal and in or through the syringe barrel distal closed end. For example, the needle distal opening can be in a distal seal at the syringe barrel distal closed end (e.g., the needle distal opening can be blocked by the distal seal) or distal to the distal seal and/or the syringe barrel distal closed end, while the needle body opening is proximal to the floating seal (e.g., as shown in FIGS. 3D, 6b1), in the floating seal (e.g., the needle body opening can be blocked by the floating seal as shown in FIGS. 3D, 6b2), or in the flowable composition lumen (e.g., as shown in FIGS. 3D, 6b3), and then the needle can be advanced through the syringe barrel distal closed end and exposing the needle distal opening for puncturing a tissue.

Optionally, the pre-filled injection device or system comprises a state wherein the flowable composition lumen, the needle body opening, and the needle distal opening are in fluidic communication. For example, in a fluidic communication state, the needle body opening can be proximal to the floating seal, while the needle distal opening is distal to the floating seal and in the flowable composition lumen. In the fluidic communication state, the needle and/or the floating seal can be moved. For example, the floating seal can be moved under the elastic resilience between the floating seal and the actuation member (e.g., pressing element) such as that the floating seal seals or blocks the needle body opening, thereby preventing or terminating discharge of the flowable composition (such as a gel) from the needle body opening and/or from the needle distal opening.

Optionally, in the fluidic communication state, the floating seal can seal the needle body opening when it moves forward and contacts the syringe barrel distal closed end, thereby preventing or terminating discharge of the flowable composition (such as a gel) from the needle body opening and/or from the needle distal opening.

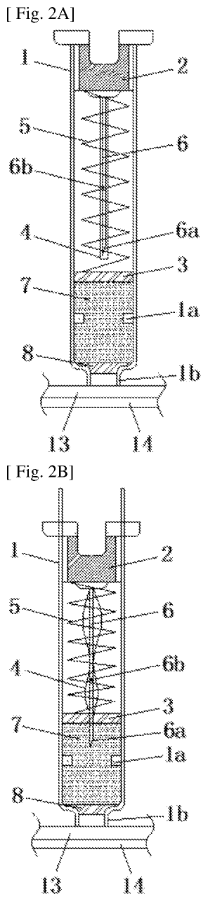

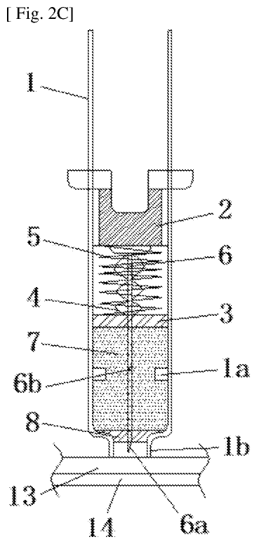

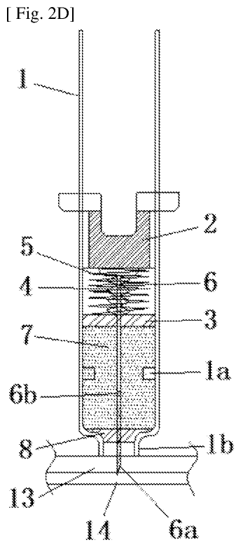

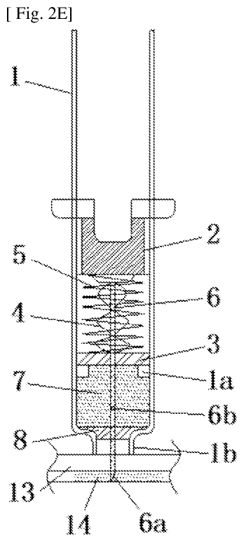

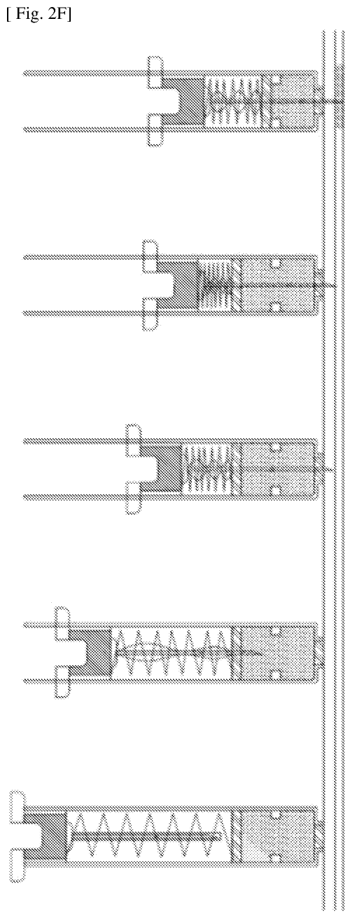

Optionally, a stopper such as an axial stopper can be provided inside the syringe lumen, distal to the floating seal. In some embodiments, the stopper can be used to limit the forward movement of the floating seal. In some embodiments, the pre-filled injection device or system comprises a fluidic communication state, wherein the flowable composition lumen is connected to the needle body opening and the needle distal opening. When the medical puncturing device is in the fluidic communication state, the needle body opening can be at the distal end of the stopper (e.g., as shown in FIG. 2D), and the floating seal can move forward due to the elastic engagement with the actuation member (e.g., pressing element).

Optionally, the pre-filled injection device or system comprises a manual control element, which is attached to the floating seal and is extended outside of the syringe barrel.

Optionally, the pre-filled injection device or system comprises a pre-puncture state after the hollow puncture needle pierces the syringe barrel distal closed end, a surface tissue puncture state, and a fluidic communication state after the puncture. In the pre-puncture state, the surface tissue puncture state, and the fluidic communication state, the length range of the hollow puncture needle extended outside of the syringe barrel distal closed end can correspond to a pre-puncture length range, a surface tissue puncture length range, and a fluidic communication length range, respectively, wherein: when the length of the of the hollow puncture needle extended outside of the syringe barrel distal closed end is within the pre-puncture length range, the needle body opening remains above the flowable composition lumen (e.g., the needle body opening can be proximal to and within the floating seal) ; and/or when the length of the of the hollow puncture needle extended outside of the syringe barrel distal closed end is within the surface tissue puncture length range, at least part of the needle body opening is connected to the flowable composition lumen; and/or, when the length of the of the hollow puncture needle extended outside of the syringe barrel distal closed end is within the fluidic communication length range, the needle body opening is positioned within the flowable composition lumen.

Optionally, an axially extended circular contacting element is formed at the syringe barrel distal closed end, wherein the difference between the upper and lower limits of the pre-puncture length range equals to the axial length of the circular contacting element.

Optionally, the elastic movement unit comprises a elastic sheath covering the outside of the hollow puncture needle. When the needle body opening is proximal to the floating seal, the elastic sheath can seal the needle body opening. In some embodiments, when the flowable composition is a gel, it may not be necessary to seal the needle body opening when it is proximal to the floating seal.

Optionally, the pre-filled injection device or system comprises a catheter guiding structure which is used to thread the catheter into a cavity (e.g., a needle body passageway connected to the needle distal opening and/or the needle body opening) of the hollow puncture needle.

Optionally, the catheter guiding structure comprises an angled guiding groove which is formed on the floating seal and extends towards the hollow puncture needle in an angle.

Optionally, the angled guiding groove is set to be through the floating seal in the front and back direction. In some embodiments, the catheter guiding structure further comprises a one-way valve which is embedded in the angled guiding groove and can be opened and closed, and/or a guiding groove plug inserted in the angled guiding groove.

Optionally, the angled guiding groove is set to be on the upper surface of the floating seal and is a non-through groove.

Optionally, the needle body opening is formed as an angled opening which opens obliquely backwards.

Optionally, the catheter guiding structure comprises an angled guiding needle hole formed on the body wall of the hollow puncture needle and opens obliquely backwards. In some embodiments, the pre-filled injection device or system comprises a fluidic communication state wherein the flowable composition lumen is in connection with the needle body opening and the needle distal opening. In the fluidic communication state, the angled guiding needle hole is positioned proximal to the floating seal.

Optionally, the catheter guiding structure further comprises a one-way valve which is embedded in the angled guiding needle hole and can be opened and closed, or a guiding groove plug inserted in the angled guiding needle hole.

Optionally, the catheter guiding structure comprises a puncturable central guiding groove that is formed on the center of the proximal surface of the actuation member (e.g., pressing element). In some embodiments, a needle proximal opening is formed on the hollow puncture needle and the needle proximal opening is set to axially align with the central guiding groove.

Optionally, the pre-filled injection device or system comprises a puncture control module and a fluid storage module that are independently manufactured and formed, wherein: the puncture control module comprises a first syringe unit and the elastic movement unit and the hollow puncture needle provided inside the first syringe unit; the fluid storage module comprises a second syringe unit, the flowable composition lumen formed inside the barrel of the second syringe unit, and a pre-filled drug composition or implant, and a module packaging component which is removably packaged to the proximal end of the second syringe unit; and a removable connection structure is formed between the first syringe unit and the second syringe unit.

In a second aspect, the present disclosure provides a medical apparatus assembly. In some embodiments, the medical apparatus assembly comprises a catheter and the medical puncturing device comprising a catheter guiding structure.

Optionally, the medical apparatus assembly further comprises a hollow auxiliary guiding needle which is matched to use with the catheter guiding structure. In some embodiments, when the auxiliary guiding needle is connected to the catheter guiding structure, the catheter can sequentially go through the needle body passageway of the auxiliary guiding needle and the catheter guiding structure and be threaded into the needle body passageway of the hollow puncture needle.

In some embodiments, when using the pre-filled injection device or system of the present disclosure, a user can first apply pressure to the actuation member (e.g., pressing element) to drive the hollow puncture needle sequentially through the floating seal and the syringe barrel distal closed end. When the needle distal opening of the hollow puncture needle reaches apparent or potential tissue gaps, cavity systems, and vessels, the needle body opening has already been positioned in the flowable composition lumen, and the floating seal has already formed an elastic engagement with the actuation member (e.g., pressing element). In some embodiments, the fluid pressure in the flowable composition lumen can be made higher than the pressure inside the an apparent or potential tissue void, cavity, or vessel.

At this time, the pre-filed fluid inside the flowable composition lumen can flow into the an apparent or potential tissue void, cavity, or vessel through the needle body opening and the needle distal opening. During the injection process, just by maintaining the position of the actuation member (e.g., pressing element), under the action of the elastic engagement between the floating seal and the actuation member (e.g., pressing element), the fluid inside the flowable composition lumen can flow into the needle body opening (and then through the needle body passageway and out of the needle distal opening), thereby achieving injection, penetration, and/or expansion of the an apparent or potential tissue void, cavity, or vessel. Additionally, the medical apparatus assembly as describe in the present disclosure can achieve implantation of catheter and other medical device through the medical puncturing device, e.g., through a catheter guiding structure and a cavity of the needle described herein.

In some embodiments, before the hollow puncture needle pierces into an apparent or potential tissue void, cavity, or vessel, the external pressure on the needle distal opening is higher than the fluid pressure in the flowable composition lumen, thus fluid cannot flow out of the needle distal opening. Thus, by observing whether the floating seal moves forward due to the elastic engagement with the actuation member (e.g., pressing element), it is possible to determine whether the hollow puncture needle has already pierced into an apparent or potential tissue void, cavity, or vessel, thereby reminding the operator of the current punctuation depth to ensure accurate puncture. Since the injection is controlled by fluid pressure changes in the flowable composition lumen, the injection process does not require an operator to manually apply thrust or force during the injection process, thus fluctuations in the flow speed can be prevented and stable injection can be achieved.

### III. Drug Compositions of Pre-Filled Injection Device or Systems

In some aspects, provided herein is a pre-loaded or pre-filled injection device or systems that comprises any of the injection devices and any of the drug compositions described herein. In some aspects, provided herein is a pre-loaded or pre-filled injection device or systems that comprises any of the injection devices and any of the implants described herein. In some aspects, provided herein is a pre-loaded or pre-filled injection device or systems that comprises any of the injection devices, any of the drug composition, and any of the implants described herein.

It should be noted that the present disclosure encompasses any combinations of any embodiments of the injection device and any embodiments of the drug composition and/or any embodiments of the implants described herein. It should also be noted that the present disclosure encompasses the combination of any embodiments of the drug compositions and any embodiments of the implants described herein, provided that they are pharmaceutically compatible. For example, in some embodiments, the pre-filled injection device or system in the present disclosure may comprise an injection device and a drug composition described herein. For another example, in some embodiments, the pre-filled injection device or system in the present disclosure may comprise an injection device, a drug composition described herein, and an implant described herein. In some embodiments, the drug composition is a fluid and the implant is contained within the fluid. In some embodiments, the drug composition is coated on or carried by the implant. In some embodiments, the implant is not coated with or does not carry any drug compositions.

In some aspects, provided herein is a pre-loaded or pre-filled injection device or systems that comprises any of the injection devices described herein and a drug composition, wherein the drug composition can be contained in any of the lumens in any of the injection devices described herein. In some embodiments, the drug composition comprises only one type of drug. In some embodiments, the drug composition comprises more than one types of drugs. In some embodiments, the drug composition comprises only one type of drug, wherein the drug is contained in a single lumen. In some embodiments, the drug composition comprises only one type of drug, wherein the drug is contained in multiple lumens. In some embodiments, the drug composition comprises more than one types of drugs, wherein the drugs are contained in a single lumen. In some embodiments, the drug composition comprises more than one types of drugs, wherein the drugs are contained in separated lumens.

In some embodiments, the drug composition comprises corticosteroids. Exemplary corticosteroids include, but are not limited to, dexamethasone, triamcinolone acetonide, triamcinolone, triamcinolone acetonide acetate, fluocinolone acetonide, prednisolone, loteprednol, difluprednate, fluorometholone, and any combination thereof. In some embodiments, the drug composition comprises a single type of corticosteroids. In some embodiments, the drug composition comprises a combination of more than one types of corticosteroids.

In some embodiments, the drug compositions comprises a formulation of corticosteroid, which comprises corticosteroids, or a pharmaceutically acceptable salt or solvate thereof, and one or more pharmaceutically acceptable excipients. In some embodiments, the formulation comprises: (1) triamcinolone; (2) hyaluronic acid, or pharmaceutically acceptable derivatives, salts, or solvates thereof; (3) a buffer agent; and (4) a tonicity agent. In some embodiments, the formulation comprises: (1) triamcinolone acetonide; (2) hyaluronic acid, or pharmaceutically acceptable derivatives, salts, or solvates thereof; (3) a buffer agent; and (4) a tonicity agent. In some embodiments, the formulation comprises: (1) triamcinolone acetonide acetate; (2) hyaluronic acid, or pharmaceutically acceptable derivatives, salts, or solvates thereof; (3) a buffer agent; and (4) a tonicity agent. In some embodiments, the formulation comprises: (1) dexamethasone; (2) hyaluronic acid, or pharmaceutically acceptable derivatives, salts, or solvates thereof; (3) a buffer agent; and (4) a tonicity agent. In some embodiments, the formulation comprises: (1) fluocinolone acetonide; (2) hyaluronic acid, or pharmaceutically acceptable derivatives, salts, or solvates thereof; (3) a buffer agent; and (4) a tonicity agent. In some embodiments, the formulation comprises: (1) prednisolone; (2) hyaluronic acid, or pharmaceutically acceptable derivatives, salts, or solvates thereof; (3) a buffer agent; and (4) a tonicity agent. In some embodiments, the formulation comprises: (1) loteprednol; (2) hyaluronic acid, or pharmaceutically acceptable derivatives, salts, or solvates thereof; (3) a buffer agent; and (4) a tonicity agent. In some embodiments, the formulation comprises: (1) difluprednate; (2) hyaluronic acid, or pharmaceutically acceptable derivatives, salts, or solvates thereof; (3) a buffer agent; and (4) a tonicity agent. In some embodiments, the formulation comprises: (1) fluorometholone; (2) hyaluronic acid, or pharmaceutically acceptable derivatives, salts, or solvates thereof; (3) a buffer agent; and (4) a tonicity agent.

In some embodiments, which can be combined with any embodiments above or below, the formulation comprises hyaluronic acid, or pharmaceutically acceptable derivatives, salts, or solvates thereof. In some embodiments, the formulation comprises a salt of hyaluronic acid. In some embodiments, the salt of hyaluronic acid is an alkali salt of hyaluronic acid, an alkaline salt of hyaluronic acid, or a combination thereof. In some embodiments, the salt of hyaluronic acid is a sodium salt of hyaluronic acid. In some embodiments, the molecular weight of the hyaluronic acid, or pharmaceutically acceptable derivatives, salts, or solvates thereof is in a range of about 50,000 Dalton to about 2,000,000 Dalton. For example, the molecular weight of the hyaluronic acid, or pharmaceutically acceptable derivatives, salts, or solvates thereof is about 50000, 60000, 70000, 80000, 90000, 100000, 110000, 120000, 130000, 140000, 150000, 160000, 170000, 180000, 190000, 200000 Dalton, or any intervening value thereof.

In some embodiments, which can be combined with any embodiments above or below, the formulation comprises a buffer agent. In some embodiments, the buffer agent comprises acetate buffers, citrate buffers, phosphate buffers, borate buffers, or any mixture thereof. In some embodiments, the buffer agent comprises phosphate buffers. In some embodiments, the buffer agent comprises disodium hydrogen phosphate, sodium dihydrogen phosphate, or a mixture thereof. In some embodiments, the buffer agent comprises sodium dihydrogen phosphate in the form of monohydrate and disodium hydrogen phosphate in the form of dodecahydrate. In some embodiments, the amount of the buffer agent is sufficient to maintain a pH value of the formulation in a range of about 5.5 to about 9.0. In some embodiments, the amount of the buffer agent is sufficient to maintain a pH value of the formulation in a range of about 6.0 to about 8.5. In some embodiments, the amount of the buffer agent is sufficient to maintain a pH value of the formulation in a range of about 6.5 to about 8.0. In some embodiments, the amount of the buffer agent is sufficient to maintain a pH value of the formulation in a range of about 7.0 to about 9.5. In some embodiments, the amount of the buffer agent is sufficient to maintain a pH value of the formulation at about 5.5, about 6.0, about 6.5, about 7.0, about 7.5, about 8.0, about 8.5, about 9.0, or any intervening value thereof. In some embodiments, the formulation further comprises a pH adjusting agent. In some embodiments, the formulation further comprises a pH adjusting agent, wherein the pH adjusting agent is sodium hydroxide. In some embodiments, the pH adjusting agent is sodium hydroxide solution, wherein the sodium hydroxide solution has a concentration of sodium hydroxide in a range about 1.5-2.5 mol/L (e.g., 2.0 mol/L).

In some embodiments, which can be combined with any embodiments above or below, the formulation comprises a tonicity agent. In some embodiments, the tonicity agent comprises sodium chloride, potassium chloride, magnesium chloride, calcium chloride, or any mixture thereof. In some embodiments, the tonicity agent comprises sodium chloride, potassium chloride, or any mixture thereof. In some embodiments, the tonicity agent comprises sodium chloride. In some embodiments, the tonicity agent is sodium chloride. In some embodiments, the amount of tonicity agent is sufficient to maintain an osmolality in a range of about 200 mOsm/kg to about 400 mOsm/kg. In some embodiments, the amount of tonicity agent is sufficient to maintain an osmolality in a range of about 250 mOsm/kg to about 300 mOsm/kg. In some embodiments, the amount of tonicity agent is sufficient to maintain an osmolality of about 200, 210, 220, 230, 240, 250, 260, 270, 280, 290, 300, 310, 320, 330, 340, 350, 360, 370, 380, 390, 400 mOsm/kg, or any intervening value thereof.

In some embodiments, which can be combined with any embodiments above or below, the formulation comprises water. In some embodiments, the water is water for injection.

In some embodiments, which can be combined with any embodiments above or below, the formulation comprises a corticosteroid, wherein the weight ratio of corticosteroid to the entire formulation is about 1.0-8.0% (w/w). In some embodiments, the formulation comprises triamcinolone acetonide, wherein the weight ratio of triamcinolone acetonide to the entire formulation is about 1.0-8.0% (w/w). For example, the weight ratio of triamcinolone acetonide to the entire formulation is about 1.0, 1.5, 2.0, 2.5, 3.0, 3.5 4.0, 4.5, 5.0, 5.5, 6.0, 6.5, 7.0, 7.5, 8.0% (w/w), or any intervening value thereof.

In some embodiments, which can be combined with any embodiments above or below, the formulation comprises hyaluronic acid, or pharmaceutically acceptable derivatives, salts, or solvates thereof, wherein the weight ratio of hyaluronic acid, or pharmaceutically acceptable derivatives, salts, or solvates thereof to the entire formulation is about 0.1-5.0%(w/w). In some embodiments, the formulation comprises triamcinolone acetonide and hyaluronic acid, or pharmaceutically acceptable derivatives, salts, or solvates thereof, wherein the weight ratio of hyaluronic acid, or pharmaceutically acceptable derivatives, salts, or solvates thereof to the entire formulation is about 0.1-5.0% (w/w). For example, the weight ratio of hyaluronic acid, or pharmaceutically acceptable derivatives, salts, or solvates thereof to the entire formulation is about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.50, 0.55, 0.60, 0.65, 0.70, 0.75, 0.80, 0.85, 0.90, 0.95, 1.00, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00% (w/w), or any intervening value thereof.

In some embodiments, which can be combined with any embodiments above or below, the formulation comprises a buffer agent, wherein the weight ratio of the buffer agent to the entire formulation is about 0.05-0.8% (w/w). In some embodiments, the formulation comprises triamcinolone acetonide and a buffer agent, wherein the weight ratio of the buffer agent to the entire formulation is about 0.05-0.8% (w/w). For example, the weight ratio of the buffer agent to the entire formulation is about 0.05, 0.10, 0.15, 0.20, 0.25, 0.30, 0.35, 0.40, 0.45, 0.50, 0.55, 0.60, 0.65, 0.70, 0.75, 0.80 % (w/w), or any intervening value thereof. In some embodiments, the buffer agent comprises disodium hydrogen phosphate and sodium dihydrogen phosphate, wherein the weight ratio of sodium dihydrogen phosphate to the entire formulation is about 0.01-0.50% (w/w), and wherein the weight ratio of disodium hydrogen phosphate to the entire formulation is about 0.05-0.20% (w/w). For example, the weight ratio of sodium dihydrogen phosphate to the entire formulation is about 0.10, 0.15, 0.20, 0.25, 0.30, 0.35, 0.40, 0.45, 0.50% (w/w), or any intervening value thereof. For example, the weight ratio of disodium hydrogen phosphate to the entire formulation is about 0.05, 0.10, 0.15, 0.20% (w/w), or any intervening value thereof.

In some embodiments, which can be combined with any embodiments above or below, the formulation comprises a tonicity agent, wherein the weight ratio of the tonicity agent to the entire formulation is about 5.0-10.0% (w/w). In some embodiments, the formulation comprises triamcinolone acetonide and a tonicity agent, wherein the weight ratio of the tonicity to the entire formulation is about 5.0-10.0% (w/w). For example, the weight ratio of the tonicity to the entire formulation is about 5.0, 5.5, 6.0, 6.5, 7.0, 7.5, 8.0, 8.5, 9.0, 9.5, 10.0% (w/w), or any intervening value.

Preferably, the weight ratio of the tonicity to the entire formulation is about 6.0-9.0% (w/w).

In some embodiments, the formulation comprises: (1) 3.0-5.0% (w/w) triamcinolone acetonide; (2) 0.1-5% (w/w) sodium salt of hyaluronic acid; (3) 0.6-0.8% (w/w) sodium chloride; (4) 0.2-0.4% (w/w) sodium dihydrogen phosphate; (5) 0.05-0.15% (w/w) disodium hydrogen phosphate; (6) sodium hydroxide sufficient to adjust pH value to about 6.5 to about 7.5; (7) water. In some embodiments, the formulation consists of: (1) 3.0-5.0% (w/w) triamcinolone acetonide; (2) 0.1-5% (w/w) sodium salt of hyaluronic acid; (3) 0.6-0.8% (w/w) sodium chloride; (4) 0.2-0.4% (w/w) sodium dihydrogen phosphate; (5) 0.05-0.15% (w/w) disodium hydrogen phosphate; (6) sodium hydroxide sufficient to adjust pH value to about 6.5 to about 7.5; (7) water.

In some embodiments, which can be combined with any embodiments above or below, the formulation is a suspension.

In some embodiments, which can be combined with any embodiments above or below, the formulation comprises triamcinolone acetonide, wherein the volume mean diameter (VMD) of triamcinolone acetonide in the formulation is about 0.5-3.5 μm. For example, the VMD of triamcinolone acetonide in the formulation is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 μm, or any intervening value thereof. In some embodiments, the D₁₀ of triamcinolone acetonide in the formulation is about 0.4-1.0 μm. For example, the D₁₀ of triamcinolone acetonide in the formulation is 0.4, 0.5, 0.6, 0.7, 0.8, 0.9, 1.0 μm, or any intervening value thereof. In some embodiments, the D₅₀ of triamcinolone acetonide in the formulation is about 1.0-2.0 μm. For example, the D 50 of triamcinolone acetonide in the formulation is 1.0, 1.1, 1.2, 1.3, 1.4, 1.5, 1.6, 1.7, 1.8, 1.9, 2.0 μm, or any intervening value thereof. In some embodiments, the D₉₀ of triamcinolone acetonide in the formulation is about 2.0-3.8 μm. For example, the D₉₀ of triamcinolone acetonide in the formulation is 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8 μm, or any intervening value thereof.

In some embodiments, the drug composition comprises a formulation of triamcinolone acetonide, wherein the formulation is a suspension of triamcinolone acetonide particles, and wherein D₁₀ of triamcinolone acetonide in the formulation is about 0.4-1.0 μm, D₅₀ of triamcinolone acetonide in the formulation is about 1.0-2.0 μm, and D₉₀ of triamcinolone acetonide in the formulation is about 2.0-3.8 μm. In some embodiments, the formulation is a suspension of triamcinolone acetonide particles, wherein D₁₀ of triamcinolone acetonide in the formulation is about 0.6-0.85 μm, D₅₀ of triamcinolone acetonide in the formulation is about 1.5-1.8 μm, and D₉₀ of triamcinolone acetonide in the formulation is about 3.0-3.5 μm. In some embodiments, the formulation is a suspension of triamcinolone acetonide particles, wherein D₁₀ of triamcinolone acetonide in the formulation is about 0.7-0.8 μm, D₅₀ of triamcinolone acetonide in the formulation is about 1.6-1.8 μm, and D₉₀ of triamcinolone acetonide in the formulation is about 3.1-3.5 μm.

In some embodiments, the drug composition comprises a formulation of triamcinolone acetonide, wherein the formulation is a suspension of triamcinolone acetonide particles, and wherein about 10% of the triamcinolone acetonide particles have a volume mean diameter (VMD) less than 0.4-1.0 μm, about 50% of the triamcinolone acetonide particles have a VMD less than 1.0-2.0 μm, and about 90 % of the triamcinolone acetonide particles have a VMD less than 2.0-3.8 μm. For one example, when D₁₀ of triamcinolone acetonide in the formulation is about 0.4 μm, D₅₀ is about 1.0 μm, and D₉₀ is about 2.0-3.8 μm, 10% of the triamcinolone acetonide particles in the formulation have a VMD less than 0.4 μm, 50% of the triamcinolone acetonide particles in the formulation have a VMD less than 1.0 μm, and 90% of the triamcinolone acetonide particles in the formulation have a VMD less than 2.0 μm. For another example, when D₁₀ of triamcinolone acetonide in the formulation is about 1.0 μm, D₅₀ is about 2.0 μm, and D₉₀ is about 3.8 μm, 10% of the triamcinolone acetonide particles in the formulation have a VMD less than 1.0 μm, 50% of the triamcinolone acetonide particles in the formulation have a VMD less than 2.0 μm, and 90% of the triamcinolone acetonide particles in the formulation have a VMD less than 3.8 μm. In some embodiments, about 10% of the triamcinolone acetonide particles in the formulation have a VMD less than 0.6-0.85 μm, about 50% of the triamcinolone acetonide particles in the formulation have a VMD less than 1.5-1.8 μm, about 90% of the triamcinolone acetonide particles in the formulation have a VMD less than 3.0-3.5 μm, and the average VMD of the triamcinolone acetonide particles in the formulation is about 1.5-2.5 μm. In some embodiments, about 10% of the triamcinolone acetonide particles in the formulation have a VMD less than 0.7-0.8 μm, about 50% of the triamcinolone acetonide particles in the formulation have a VMD less than 1.6-1.8 μm, about 90% of the triamcinolone acetonide particles in the formulation have a VMD less than 3.1-3.5 μm, and the average VMD of the triamcinolone acetonide particles in the formulation is about 1.8-2.0 μm.

In some embodiments, the drug composition comprises a formulation of triamcinolone acetonide, wherein the formulation is a suspension of triamcinolone acetonide particles, and wherein about 10% of the triamcinolone acetonide particles have a VMD less than about 0.5 μm to about 0.85 μm, about 50% of the triamcinolone acetonide particles have a VMD less than about 1.2 μm to about 1.9 μm, and about 90% of the triamcinolone acetonide particles have a VMD less than about 2.5 μm to about 3.6 μm. In some embodiments, about 10% of the triamcinolone acetonide particles have a VMD less than about 0.7 μm to about 0.8 μm, about 50% of the triamcinolone acetonide particles have a VMD less than about 1.5 μm to about 1.8 um, and about 90% of the triamcinolone acetonide particles have a VMD less than about 3.0 μm to about 3.5 μm. In some embodiments, about 10% of the triamcinolone acetonide particles have a VMD less than about 0.75 μm to about 0.78 μm, about 50% of the triamcinolone acetonide particles have a VMD less than about 1.63 μm to about 1.79 μm, and about 90% of the triamcinolone acetonide particles have a VMD less than about 3.15 μm to about 3.48 μm. In some embodiments, the average VMD of the triamcinolone acetonide particles in the formulation is about 0.5-2.5 μm. In some embodiments, the average VMD of the triamcinolone acetonide particles in the formulation is about 0.7-2.1 μm. In some embodiments, the average VMD of the triamcinolone acetonide particles in the formulation is about 0.8-2.0 μm. In some embodiments, the average VMD of the triamcinolone acetonide particles in the formulation is about 0.81-1.99 μm.

In some embodiments, which can be combined with any of the embodiments above or below, the drug composition comprises a formulation of triamcinolone acetonide, wherein the formulation is prepared by a method comprising wet milling. In some embodiments, the formulation is prepared by a method comprising ball milling. For example, the formulation is prepared by a method comprising adding hyaluronic acid, for example, in the form of an aqueous solution, to a mixture comprising triamcinolone acetonide particles. For another example, the formulation is prepared by a method comprising adding triamcinolone acetonide particles into a mixture comprising hyaluronic acid, for example, an aqueous solution of hyaluronic acid, and milling the triamcinolone acetonide particles in the mixture comprising hyaluronic acid, until the VMD of the triamcinolone acetonide particles are within a desired range or value (e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 μm, or any intervening value thereof).

In some embodiments, the drug composition comprises a formulation of triamcinolone acetonide, wherein the formulation is substantially free of triamcinolone acetonide particles with a VMD of less than 0.2 μm. In some embodiments, the formulation is substantially free of triamcinolone acetonide particles with a VMD of 0.2-0.4 μm. In some embodiments, in the formulation, more than 10%, more than 50 %, or more than 90% of triamcinolone acetonide particles have a VMD of more than 0.2 μm, 0.3 μm, 0.4 μm, more than 0.5 μm, more than 0.6 μm, more than 0.7 μm, more than 0.8 μm, more than 0.9 μm, more than 1.0 μm, more than 1.5 μm, more than 2.0 um, more than 2.5 μm, more than 3.0 μm, or more than 3.8 μm. In some embodiments, in the formulation, no less than about 10% of triamcinolone acetonide particles have a VMD of more than 3.8 μm. In some embodiments, in the formulation, no less than about 50% of triamcinolone acetonide particles have a VMD of more than 2.0 μm.

In some embodiments, in the formulation, no less than about 90% of triamcinolone acetonide particles have a VMD of more than 1.0 μm. In some embodiments, in the formulation, no less than about 10% of triamcinolone acetonide particles have a VMD of more than 2.0 μm, no less than about 50% of triamcinolone acetonide particles have a VMD of more than 1.0 μm, and no less than about 90% of triamcinolone acetonide particles have a VMD of more than 0.4 μm.

In some embodiments, the wetting agent used herein comprises polysorbate 80. In some embodiments, the wetting agent used herein is polysorbate 80. In some embodiments, the drug composition comprises a formulation of triamcinolone acetonide, wherein the formulation is free of polysorbate 80, any derivatives thereof, or any analogs thereof.

In some embodiments, which can be combined with any of the embodiments above or below, the drug composition comprises a formulation of triamcinolone acetonide, wherein the formulation further comprises one or more viscosity agents.

In some aspects, provided herein is a pre-loaded or pre-filled injection device or systems that comprises any of the injection devices described herein and a drug composition, wherein the drug composition comprises tyrosine kinase inhibitors. Exemplary tyrosine kinase inhibitors include, but are not limited to, axitinib, afatinib, erlotinib, gefitinib, crizotinib, dabrafenib, vemurafenib, dasatanib, imatinib, nilotinib, trametinib, or any combination thereof. In some embodiments, the drug composition comprises axitinib.

In some aspects, provided herein is a pre-loaded or pre-filled injection device or systems that comprises any of the injection devices described herein and a drug composition, wherein the drug composition comprises complement inhibitors. In some embodiments, the drug composition comprises plasma kallikrein inhibitors.

In some aspects, provided herein is a pre-loaded or pre-filled injection device or systems that comprises any of the injection devices described herein and a drug composition, wherein the drug composition comprises neuroprotective agents. Exemplary neuroprotective agents include, but are not limited to, cholic acid, chenodeoxycholic acid, deoxycholic acid, glycocholic acid, glycochenodeoxycholic acid, glycodeoxycholic acid, lithocholic acid, taurocholic acid, taurochenodeoxycholic acid, taurodeoxycholic acid, tauroursodeoxycholic acid, ursodeoxycholic acid, or any combination thereof. In some embodiments, the drug composition comprises a neuroprotective agent, wherein the neuroprotective agent is tauroursodeoxycholic acid.

In some aspects, provided herein is a pre-loaded or pre-filled injection device or systems that comprises any of the injection devices described herein and a drug composition, wherein the drug composition comprises hypoxia factor-inducible inhibitors. Exemplary hypoxia factor-inducible inhibitors include, but are not limited to, EZN-2698, aminoflavone, camptothecins (e.g., topotecan, EZN-2208, SN38, irinotecan, temsirolimus, everolimus, sirolimus, LY294002, wortmannin, cardiac glycosides, digoxin, ouabain, proscillaridin, 2ME2's, romidepsin (KF228), trichostatin, LW6, acriflavine, echinomycin, anthracyclines (e.g., doxorubicin and daunorubicin), chetomin, bortezomib, or any combination thereof. In some embodiments, the drug composition comprises a hypoxia factor-inducible inhibitor, wherein the hypoxia factor-inducible inhibitor is acriflavine.

In some aspects, provided herein is a pre-loaded or pre-filled injection device or systems that comprises any of the injection devices described herein and a drug composition, wherein the drug composition comprises adrenergic receptor agonists. Exemplary adrenergic receptor agonists include, but are not limited to, adrenaline, noradrenaline, isoprenaline, dopamine, phenylephrine, methoxamine, midodrine, oxymetazoline, α-methyldopa, clonidine, brimonidine, dobutamine, salbutamol/albuterol, terbutaline, salmeterol, formoterol, pirbuterol, clenbuterol, or any combination thereof. In some embodiments, the drug composition comprises an adrenergic receptor agonist, wherein the adrenergic receptor agonist is brimonidine.

In some aspects, provided herein is a pre-loaded or pre-filled injection device or systems that comprises any of the injection devices described herein and a drug composition, wherein the drug composition comprises one or more agents for gene therapy, such as one or more viral vectors and/or non-viral gene therapy vectors.

Exemplary gene therapy drugs include, but are not limited to, gene therapies using AAV2, AAV5, AAV8, or AAV9 vectors, gene therapies using ET, liposomes, or DNA nanoparticles as vectors, or any combination thereof. For instance, electroporation or electropermeabilization is a physical method of introducing polar molecules such as DNA into eukaryotic cells through the cell membrane by exposing cells to electric pulses, and can be used in gene therapies.

In some aspects, provided herein is a pre-loaded or pre-filled injection device or systems that comprises any of the injection devices described herein and a drug composition, wherein the drug composition comprises one or more protein or polypeptide drugs. Exemplary protein and polypeptide drugs include, but are not limited to, anti-VEGF drugs (e.g., bevacizumab, ranibizumab, aflibercept, conbercept, etc.), bispecific antibody drugs (e.g., Faricimab), vasoconstriction agents (e.g., endothelin-1), TNF-α inhibitors (e.g., adalimumab), or any combination thereof.

In some aspects, provided herein is a pre-loaded or pre-filled injection device or systems that comprises any of the injection devices described herein and a drug composition, wherein the drug composition comprises one or more therapeutic cells or therapeutic components (e.g., cell components) for cell therapy. Exemplary cells or therapeutic components include, but are not limited to, stem cells, Treg cells, exosomes, or any combination thereof.

In some aspects, provided herein is a pre-loaded or pre-filled injection device or systems that comprises any of the injection devices described herein and a drug composition, wherein the drug composition comprises gel or polymer aqueous solution, such as one or more viscoelastic materials. Examples of gel or polymer aqueous solution that can be used herein include, but are not limited to sodium hyaluronate, Provisc (1% viscous and transparent material which is a specific fraction of sodium hyaluronate), Viscoat (a dispersive viscoelastic comprising of sodium hyaluronate and chondroitin sulphate), Amvisc (a purified fraction of sodium hyaluronate), Amvisc Plus (a 1.6% sodium hyaluronate product derived from rooster combs), sodium chondroitin sulfate/sodium hyaluronate, or DisCo Visc (4% sodium chondroitin sulfate, 1.65% sodium hyaluronate), sodium carboxymethyl cellulose, poloxamer, or any combination thereof.

In some aspects, provided herein is a pre-loaded or pre-filled injection device or systems that comprises any of the injection devices described herein and a drug composition, wherein the drug composition comprises one or more antitumor drugs. Exemplary antitumor drugs include but are not limited to paclitaxel, immunosuppressive agents (e.g., ipilimumab), or any combination thereof.

In some aspects, provided herein is a pre-loaded or pre-filled injection device or systems that comprises any of the injection devices described herein and a drug composition, wherein the drug composition comprises one or more herbal medicines. Exemplary herbal medicines include, but are not limited to, artemisinin, curcumin, pilocarpine, or any combination thereof.

In some aspects, provided herein is a pre-loaded or pre-filled injection device or systems that comprises any of the injection devices described herein and a drug composition, wherein the drug composition comprises one or more H1 receptor antagonists. Exemplary H1 receptor antagonists include, but are not limited to, 0.3% pheniramine maleate (naproxen), emestine (imatin) and 0.05% levocabastine hydrochloride (rivastine) or any combination thereof.

In some aspects, provided herein is a pre-loaded or pre-filled injection device or systems that comprises any of the injection devices described herein and a drug composition, wherein the drug composition comprises one or more mast cell stabilizers. Exemplary mast cell stabilizers include, but are not limited to, 4% sodium cromoglycate (Crolom), 2% nedocromil (Alocril), 0.1% pirolast (Alamast), 0.1% lodoxamide tromethamine (Alomide), or any combination thereof.

In some aspects, provided herein is a pre-loaded or pre-filled injection device or systems that comprises any of the injection devices described herein and a drug composition, wherein the drug composition comprises one or more non-steroidal anti-inflammatory drugs (NSAIDs). Exemplary NSAIDs include, but are not limited to, aspirin, ibuprofen, naproxen, celecoxib, 0.5% ketorolac tromethamine (Acular), or any combination thereof.

In some aspects, provided herein is a pre-loaded or pre-filled injection device or systems that comprises any of the injection devices described herein and a drug composition, wherein the drug composition comprises one or more prostaglandin derivatives. Exemplary prostaglandin derivatives include, but are not limited to, latanoprost, travoprost, bimeprost or any combination thereof.

In some aspects, provided herein is a pre-loaded or pre-filled injection device or systems that comprises any of the injection devices described herein and a drug composition, wherein the drug composition comprises one or more anticholinergic drugs. Exemplary anticholinergic drugs include, but are not limited to, atropine, homatropine, tropicamide, or any combination thereof.

In some aspects, provided herein is a pre-loaded or pre-filled injection device or systems that comprises any of the injection devices described herein and a drug composition, wherein the drug composition comprises one or more anesthesia agents. Exemplary anesthesia agents include, but are not limited to, tetracaine, obucaine, proparacaine, or any combination thereof.

### IV. Adapters for Syringes

In some aspects, provided herein is a set of adapters that can be combined with a syringe to significantly improve the precision of inject depth of the syringe, facilitate injection of the medicament into tissues such as an ocular tissue, and/or can facilitate the implant of certain structures in tissues such as an ocular tissues. In some aspects, the adapters described herein can improve the injection precision and safety of a syringe, once installed onto the syringe. In some embodiments, the adapters described herein can improve the injection precision and safety of other syringes, for example, the syringe disclosed in US 2020/0069883, once installed onto the syringe.

In some aspects, the set of adapters comprises:
a contact member extending from a proximal end to a distal end;
and a pressing unit comprising a first elastic element;
wherein the contact member can be installed to the needle distal end of a syringe such that the distal end of the contact member is distal to the needle distal end opening, and the distal end of the contact member can directly contact with surface tissues at a target injection site; and
wherein the pressing unit can be installed to engage a syringe barrel and push shaft such that the pressing unit elastically engages the push shaft and/or the syringe barrel via the first elastic element.

In some embodiments, the pressing unit can be assembled to a syringe, wherein the pressing unit elastically engages the push shaft and/or the syringe barrel via the first elastic element. In some embodiments, the first elastic element takes the form of a spring (e.g., 31 in FIGS. 16A and 16B). In some embodiments, the first elastic element can apply a force on the push shaft and push the push shaft distally. In some embodiments, the pressing unit can be restrict the movement of the syringe barrel of a syringe, especially distal movement. In some embodiments, the pressing unit has a pair of stoppers (e.g., 32 in FIG. 16A) or a locking element (e.g., 33 in FIG. 16B), and after assembling the pressing unit to a syringe, and the stoppers or locking element can prevent the movement of the syringe barrel distally.

In some embodiments, the set of adapters described herein comprises a contact member, wherein after assembling the contact member to a syringe, the proximal end of the contact member is in direct contact with the needle base or the distal end of the syringe barrel. In some embodiments, as shown in FIG. 15A, after assembling the contact member 25 to a syringe, the proximal end of the contact member is in direct contact with the distal end of the syringe barrel 1. In some embodiments, after assembling the contact member to a syringe, the proximal end of the contact member is in direct contact with the needle base. In some embodiments, as illustrated in FIG. 15A, after assembling the contact member to a syringe, the proximal end of the contact member is in direct contact with the needle base or the syringe barrel, and the contact member 25 is made of one or more materials with low elastic modulus (e.g., Young's modulus). In some embodiments, the contact member has a Young's modulus of about 0.001 GPa to about 15 GPa. In some embodiments, the contact member has a Young's modulus of about 0.01 GPa to about 10 GPa. In some embodiments, the contact member has a Young's modulus of about 0.1 GPa to about 5 GPa.

In some embodiments, the set of adapters described herein further comprises a second elastic element (e.g., 26 in FIG. 15C-15E), wherein after assembling the contact member and the second elastic element to a syringe, the second elastic element elastically connects the proximal end of the contact member to the needle base of the syringe or the distal end of the syringe barrel. In some embodiments of the foregoing, as illustrated in FIG. 15B, the contact element 25 has a high elastic modulus (e.g., Young's modulus), and is elastically engaged with the needle base or distal end of the syringe barrel via the second elastic element 26. In some embodiments, the contact element has a Young's modulus of more than 10 GPa. In some embodiments, the contact element has an elastic modulus larger than that of the second elastic element.

In some embodiments, the contact member comprises a first part and a second part, wherein the first part is distal to the second part. In some embodiments, the first part and the second part have different elasticity. In some embodiment, the first part is more elastic than the second part. In some embodiment, the first part is less elastic than the second part. In some embodiments, as illustrated in FIG. 15D, the contact member 25 comprises a first part 25a and a second part 25b, wherein the first part 25a is more elastic than the second part 25b, the first part 25a is distal to the second part 25b, and proximal end of the second part 25b is connected to the needle base or syringe barrel via the second elastic element 26.

In some embodiments, the set of adapters described herein further comprises a connector (e.g., 27 in FIG. 15F), wherein after assembling the contact member to a syringe, the connector connects the proximal end of the contact member to the needle base or the distal end of the syringe barrel, and wherein the connector is less elastic than the contact member. In some embodiments of the foregoing, the contact element has a low elastic modulus. In some embodiments, the contact member has a Young's modulus of about 0.001 GPa to about 10 GPa. In some embodiments, the connector has a high elastic modulus. In some embodiments, the connector has a Young's modulus of more than 10 GPa.

In some embodiments, the contact member takes the form of a sheath or a sleeve around the needle. In some embodiments, the contact member is an elastic sheath or sleeve around the needle (e.g., 25 in FIG. 15A). In some embodiments, the contact member takes the form of a block (e.g., 25 in FIG. 15B). In some embodiments, the contact member takes the form of a block and the distal end of the needle can pierce through the block. In some embodiments, the contact member is an elastic block and the distal end of the contact member has a surface shape adapted for surface tissues at a target injection site. For example, the distal end of the contact member may have certain patterns (e.g., FIG. 15G). For another example, the distal end of the contact member may be oblique (e.g., FIG. 15H). In some embodiments, the second elastic element takes the form of a spring (e.g., 26 in FIG. 15B and FIG. 15D). In some embodiments, the second elastic element takes the form of a sheath or a sleeve around the needle (e.g., 26 in FIG. 15C and FIG. 15E). In some embodiments, the second elastic element is an elastic sheath around the needle. In some embodiments, the connector takes the form of a sheath or a sleeve around the needle (e.g., 27 in FIG. 15F).

### V. Methods for Medical Penetration and Implant

In some embodiments, described herein are methods for medical puncture, for example, in an eye or other organs or tissues.

As shown in FIGS. 1-11B, in some embodiments, the present disclosure provides pre-filed injection device or system which comprises syringe barrel 1, an actuation unit (e.g., an elastic movement unit for pushing a needle), hollow puncture needle 6, and flowable composition lumen 7.

In some embodiments, syringe barrel 1 comprises a distal closed end and a proximal open end. In some embodiments, syringe barrel 1 can be designed to have two open ends in an axial direction, and sealing of the distal end can be achieved by installing distal seal 8 at the distal opening of syringe barrel 1. In some embodiments, distal seal 8 can be made of a material that can be punctured by hollow puncture needle 6, such as rubber or the like.

In some embodiments, the actuation unit (e.g., elastic movement unit) comprises actuation member (e.g., pressing element) 2 and floating seal 3, where the floating seal 3 sealingly engages an inside wall of the syringe barrel and is configured to move in an axial direction, e.g., toward the distal end or the proximal end of the syringe barrel. In some embodiments, actuation member (e.g., pressing element) 2 or a portion thereof is located outside the proximal opening of the syringe barrel, so that an operator can press on the actuation member (e.g., pressing element) or portion thereof manually. In some embodiments, floating seal 3 elastically engages actuation member 2, and when pressure is applied on actuation member 2, floating seal 3 can move forward or backward relative to the actuation member (e.g., pressing element). In some embodiments, floating seal 3 is configured to move toward the distal end of the syringe barrel. In some embodiments, floating seal 3 is configured to move toward the proximal end of the syringe barrel. In some embodiments, the position of the actuation member (e.g., pressing element) relative to the syringe barrel is kept still, floating seal 3 is configured to move forward (e.g., in a distal direction) under elastic resilience due to the elastic engagement with the actuation member (e.g., pressing element).

In some embodiments, hollow puncture needle 6 is fixedly connected to actuation member 2. When no pressure is applied to actuation member 2, hollow puncture needle 6 remains proximal to floating seal 3 and the two do not come into contact. In some embodiments, hollow puncture needle 6 itself comprises needle distal opening 6a and needle body opening 6b. In some embodiments, needle distal opening 6a and needle body opening 6b are connected through a needle cavity or needle body passageway of hollow puncture needle 6.

In some embodiments, flowable composition lumen 7 is used for storage, e.g., of a medication and other flowable composition such as a liquid or a gel. In some embodiments, the flowable composition lumen is enclosed by a distal closed end of the syringe barrel, a lumen wall of the syringe barrel, and floating seal 3; that is, the flowable composition lumen occupies a distal portion of a syringe barrel lumen. In some embodiments, since floating seal 3 can move along in an axial direction, flowable composition lumen 7 is configured to have a variable volume, thus the fluid pressure inside flowable composition lumen 7 can change due to an axial movement of floating seal 3. In some embodiments, the flowable composition lumen comprises a pre-filled drug composition. In some embodiments, the flowable composition lumen comprises a pre-filled fluid and/or one or more structures to be implanted in an eye, e.g., in the SCS.

In some embodiments, using a pre-filed injection device or system disclosed herein comprises applying pressure on actuation member 2, thereby advancing hollow puncture needle 6 forward in a distal direction, sequentially through floating seal 3 (e.g., by puncturing the floating seal or forcing open an existing aperture or slit through the floating seal) and through a distal closed end (e.g., by puncturing the distal closed end or forcing open an existing aperture or slit through the distal closed end) of the syringe barrel. The existing aperture or slit may be through the floating seal, e.g., from a proximal surface of the floating seal to a distal surface of the floating seal, thereby providing a through hole in the floating seal. The existing aperture or slit may be not through the entire floating seal, and advancing the needle distal end through the floating seal may comprise advancement through the existing aperture or slit and puncturing a portion of the floating seal in any suitable combination. For instance, the needle distal end may first advance through an existing aperture or slit from a proximal surface and then puncture the floating seal before emerging from a distal surface of the floating seal, or vice versa. In some embodiments, hollow puncture needle 6 pierces into an apparent or potential tissue void, cavity, or vessel, thereby placing needle distal opening 6a in the apparent or potential tissue void, cavity, or vessel. In some embodiments, needle body opening 6b is positioned inside flowable composition lumen 7, and floating seal 3 is elastically engaged with actuation member 2. In some embodiments, the fluid pressure in flowable composition lumen 7 is higher than the pressure inside the apparent or potential tissue void, cavity, or vessel.

At this time, the flowable composition inside flowable composition lumen 7 can flow through needle body opening 6b and needle distal opening 6a and into the apparent or potential tissue void, cavity, or vessel. In some embodiments, during an injection process, a user can simply maintain the pressure on actuation member 2, e.g., without further increasing the pressure. Under the action of the elastic engagement between floating seal 3 and actuation member 2, the flowable composition (e.g., a solution, a suspension, or a gel) inside flowable composition lumen 7 can enter needle body opening 6b and through the needle body passageway, thus achieving injection, penetration, and/or expansion of the apparent or potential tissue void, cavity, or vessel.

In some embodiments, before hollow puncture needle 6 pierces into an apparent or potential tissue void, cavity, or vessel, external pressure on needle distal opening 6a is higher than the fluid pressure in flowable composition lumen 7, e.g., due to the needle distal opening being in a tissue denser, harder, and/or less deformable than the apparent or potential tissue void, cavity, or vessel. Thus, the flowable composition inside the flowable composition lumen cannot exist needle distal opening 6a and into the surrounding tissue. Take the puncture process of the SCS of the eye as an example, when hollow puncture needle 6 has already pierced sclera 13 but has not yet pierced SCS 14, regardless of whether needle body opening 6b is in fluid communication with flowable composition lumen 7 or not, the flowable composition would not exit from needle distal opening 6a. This is because sclera 13 is relatively dense, and when needle distal opening 6a is inside sclera 13, a relatively high external pressure is applied on needle distal opening 6a. The external pressure is higher than the fluid pressure in flowable composition lumen 7, and the dense tissue such as the sclera essentially functions as a plug that prevents the flowable composition from flowing out.

In some embodiments, by observing whether floating seal 3 moves forward due to the elastic engagement when actuation member 2 is held still under pressure, an operator can determine whether hollow puncture needle 6 has already pierced into an apparent or potential tissue void, cavity, or vessel, thereby informing the operator of the current needle depth and/or location of the needle distal opening and ensure accurate needle placement. In some embodiments, since the injection is controlled by fluid pressure changes in flowable composition lumen 7, the injection process does not require manually applying a force that is transmitted via relatively rigid medium (e.g., solid or liquid) in order to advance and precisely place the needle tip into an apparent or potential tissue void, cavity, or vessel. Rather, an abrupt force applied to actuation member 2 can be buffered due to the elastic engagement between actuation member 2 and floating seal 3, thus allowing more controllable and steady movement of the floating seal. In some embodiments, using a device disclosed herein, fluctuations in the flow speed can be prevented or reduced and steady injection can be achieved.

In some embodiments, when hollow puncture needle 6 pierces through the syringe barrel distal closed end, the medical puncturing device can be in at least three states: a pre-puncture state, a surface tissue puncture state, and a fluidic communication state.

In some embodiments, in the pre-puncture state, the length range of hollow puncture needle 6 extending from the syringe barrel distal closed end is a pre-puncture length range. Within this range, hollow puncture needle 6 has not yet started puncturing an organism or a tissue thereof.

In some embodiments, a system or device of the present disclosure comprises a flowable composition lumen pre-filled with a flowable composition. In some embodiments, prior to use of the system or device, the needle is already through the floating seal. In some embodiments, prior to use of the system or device, the needle is already through the floating seal and the syringe barrel distal end, e.g., a distal seal sealing the syringe barrel distal end.

In some embodiments, the flowable composition is of a relatively high viscosity, e.g., higher than water-like consistency, such as a gel or paste-like material. Elastic sleeve or sheath 4 shown in the figures of the present disclosure is optional, especially when the viscosity of the flowable composition is sufficient to prevent discharge from the needle body opening and/or needle distal opening when the openings are in the flowable composition lumen. For example, as shown in FIG. 3A, the needle can be through the floating seal such that needle body opening 6b is proximal to the floating seal while needle distal opening 6a is in the flowable composition lumen. Discharge of the flowable composition from the needle body opening can be prevented due to viscosity of the composition, and the elastic sheath is optional. Alternatively, as shown in FIG. 3B, the needle body opening 6b can be in the flowable composition lumen while needle distal opening 6a is outside the flowable composition lumen. Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be outside the flowable composition lumen, while needle body opening 6b can be proximal to the floating seal (e.g., as shown in FIGS. 3C, 6b1) or within the floating seal (e.g., as shown in FIGS. 3C, 6b2). Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be within a distal seal at the syringe barrel distal closed end (e.g., the needle distal opening can be blocked by the distal seal), while needle body opening 6b can be proximal to the floating seal (e.g., as shown in FIGS. 3D, 6b1), within the floating seal (e.g., as shown in FIGS. 3D, 6b2), or within the flowable composition lumen (e.g., as shown in FIGS. 3D, 6b3). Discharge of the flowable composition from the needle distal opening and the needle body opening can be prevented.

In some embodiments, for example prior to or during the use of the system or device, needle distal opening 6a can be within the flowable composition lumen, while needle body opening 6b can be within the floating seal (e.g., as shown in FIGS. 3E, 6b1) or within the flowable composition lumen (e.g., as shown in FIGS. 3E, 6b2). Discharge of the flowable composition from the needle body opening can be prevented.

In some embodiments, for example prior to or during the use of the system or device, needle distal opening 6a can be within the floating seal, while needle body opening 6b can be proximal to the floating seal (e.g., as shown in FIGS. 3F, 6b). Discharge of the flowable composition from the needle body opening can be prevented.

In some embodiments, in the surface tissue puncture state, the length range of hollow puncture needle 6 extending from the syringe barrel distal closed end is a surface tissue puncture length range. Within this range, the distal end of hollow puncture needle 6 has entered a surface tissue (for example, pierced into sclera 13) but has not yet entered the apparent or potential tissue void, cavity, or vessel (for example, not pierced into SCS 14). In some embodiments, because the surface tissue is relatively dense, external pressure on needle distal opening 6a is higher than the fluid pressure in flowable composition lumen 7, therefore, no matter whether needle body opening 6b is connected to flowable composition lumen 7 or not, the flowable composition does not enter needle body opening 6b and/or exit needle distal opening 6a.

In some embodiments, while in the fluidic communication state, the length range of hollow puncture needle 6 extending from the syringe barrel distal closed end is the a fluidic communication. Within this range, the distal end of hollow puncture needle 6 has pierced into the apparent or potential tissue void, cavity, or vessel. In some embodiments, the device can be designed such that in the fluidic communication state, the fluid pressure in flowable composition lumen 7 is higher than the pressure inside the apparent or potential tissue void, cavity, or vessel. In some embodiments, in the fluidic communication state, needle body opening 6b has already positioned inside flowable composition lumen 7, and due to a difference in the internal (e.g., in the apparent or potential tissue void, cavity, or vessel) and external (e.g., in flowable composition lumen 7) pressures, the flowable composition inside lumen 7 can flow into the apparent or potential tissue void, cavity, or vessel through needle body opening 6b, the needle body passageway, and then needle distal opening 6a.

In some embodiments, floating seal 3 moves distally due to the elastic engagement with actuation member 2 (e.g., due to the pressure in the flowable composition lumen being higher than a backpressure at the needle distal opening in the apparent or potential tissue void, cavity, or vessel) until the floating seal seals needle body opening 6b (e.g., as shown in FIGS. 4A-4B). In some embodiments, the axial dimension of the needle body opening is no greater than the thickness of the floating seal. In some embodiments, the needle body opening can be completely sealed or blocked by the floating seal, at which time no more flowable composition exits needle distal opening 6a to enter the tissue void. In some embodiments, when the floating seal blocks the needle body opening, only a portion of the total volume of flowable composition has exited needle distal opening 6a (e.g., as shown in FIG. 4A). In some embodiments, when the floating seal blocks the needle body opening, the total volume of flowable composition in the lumen has exited needle distal opening 6a (e.g., as shown in FIG. 4B).

In some embodiments, the needle body opening can be in the distal seal or in a tissue of a subject, the flowable composition will stop existing needle distal opening 6a (e.g., as shown in FIG. 4C). In some embodiments, the distance between needle distal opening 6a and needle body opening 6b can be keep constant. In some embodiments, the distance between needle distal opening 6a and needle body opening 6b can be varied. For example, a needle having a suitable distance between needle distal opening 6a and needle body opening 6b can be selected based on a known or estimated depth of the tissue to be accessed. In some embodiments, stopper la is provided inside the syringe lumen and can be used to limit the forward movement of floating seal 3 in order to achieve precise injection, for example, injection of a pre-determined volume.

In some embodiments, once floating seal 3 contacts stopper la, further distal movement of the floating seal is limited, thereby stabilizing floating seal 3 for subsequent operation, for example, as shown in FIGS. 6-11B.

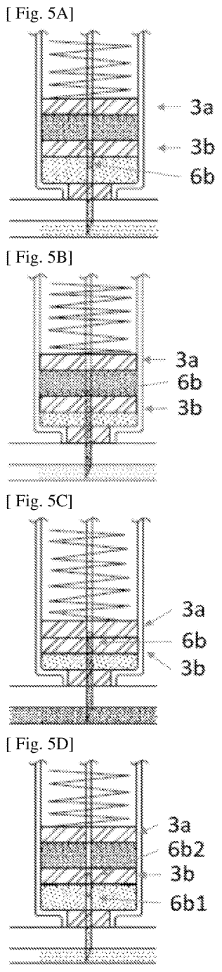

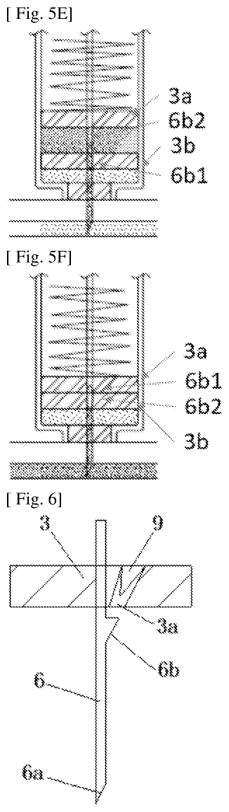

In some embodiments, a system or device disclosed herein comprises two or more floating seals. For example, as shown in FIG. 5A, a first lumen is formed between floating seal 3b and the distal seal of the syringe barrel, and a second lumen is formed between floating seal 3a and floating seal 3b. In some embodiments, the first lumen and the second lumen comprise the same flowable material. In some embodiments, the first lumen and the second lumen comprise different flowable compositions. In some embodiments, the first lumen and the second lumen comprise the same medicament (e.g., active pharmaceutical ingredient) in the same or different flowable carriers or excipients. In some embodiments, the first lumen and the second lumen comprise different medicaments (e.g., active pharmaceutical ingredients) in the same or different flowable carriers or excipients. In some embodiments, the first lumen comprises a medicament and the second lumen comprises a pharmaceutically acceptable carrier or excipient such as a saline, or vice versa. In some embodiments, which may be combined with any embodiments of the foregoing, the first lumen is pre-filled with a first flowable material. In some embodiments, which may be combined with any embodiments of the foregoing, the second lumen is pre-filled with a second flowable material. The first flowable material and the second flowable material can be the same or different. For instance, the first flowable material and the second flowable material can comprise the same active pharmaceutical ingredient (but in different carriers or excipients, for example), or can comprise different active pharmaceutical ingredients. In some embodiments, which may be combined with any embodiments of the foregoing, both the first and the second lumen is pre-filled with the same flowable material (e.g., same drug formulation) or with different flowable materials (e.g., different drug formulations).

In some embodiments, the flowable compositions in the first lumen and the second lumen can be sequentially delivered to an apparent or potential tissue void, cavity, or vessel. In some embodiments, the flowable compositions in the first lumen and the second lumen can be mixed in the apparent or potential tissue void, cavity, or vessel. In some embodiments, the flowable composition in the first lumen enters the apparent or potential tissue void, cavity, or vessel in order to access and/or expand the tissue void, cavity, or vessel. Subsequently, the flowable composition in the second lumen comprising a medicament can enter the apparent or potential tissue void, cavity, or vessel. For example, as shown in FIG. 5A, when needle distal opening 6a is in the apparent or potential tissue void, cavity, or vessel while needle body opening 6b is in the first lumen (between floating seal 3b and the distal seal of the syringe barrel), the flowable composition in the first lumen is delivered to the tissue. In FIG. 5B, needle distal opening 6a can be held still in the apparent or potential tissue void, cavity, or vessel, when floating seal 3b moves distally and needle body opening 6b contacts the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b, as shown in FIG. 5C. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, the dimension of needle body opening 6b along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the dimension of needle body opening 6b along the needle axis is no greater than the thickness of floating seal 3a and floating seal 3b combined. In some embodiments, the dimension of needle body opening 6b along the needle axis is greater than the thickness of floating seal 3b and less than the thickness of floating seal 3a and floating seal 3b combined. In some embodiments, a system or device disclosed herein comprises one or more additional floating seals (e.g., a third floating seal, 3c) that are proximal to floating seal 3a, distal to floating seal 3b, and/or between floating seal 3a and floating seal 3b, such that a third lumen is formed and a third flowable composition may be delivered before the first flowable composition, after the second flowable composition, or between the first and second flowable compositions. In some embodiments, the third lumen is pre-filled with a flowable material.

In some embodiments, a pre-filled injection device or system disclosed herein comprises two or more needle body openings. In some embodiments, a pre-filled injection device or system disclosed herein comprises two or more needle body openings and two or more floating seals. For example, as shown in FIG. 5D, when needle distal opening 6a is in the apparent or potential tissue void, cavity, or vessel while needle body opening 6b1 is in the first lumen (between floating seal 3b and the distal seal of the syringe barrel) and needle body opening 6b2 is blocked by floating seal 3b, the flowable composition in the first lumen is delivered to the tissue. In FIG. 5E, needle distal opening 6a can be held still in the apparent or potential tissue void, cavity, or vessel, when floating seal 3b moves distally to block needle body opening 6b1, allowing needle body opening 6b2 to contact the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b2 (and/or needle body opening 6b1) as shown in FIG. 5F. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, a set (e.g., predetermined) volume of the flowable composition is pre-filled in the first lumen. In some embodiments, a set (e.g., predetermined) volume of the flowable composition is pre-filled in the second lumen. In some embodiments, the distance between needle body opening 6b1 and needle body opening 6b2 along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the distance between needle body opening 6b1 and needle body opening 6b2 along the needle axis is no greater than the thickness of floating seal 3a and floating seal 3b combined. In some embodiments, the distance between needle body opening 6b1 and needle body opening 6b2 along the needle axis is greater than the thickness of floating seal 3b and less than the thickness of floating seal 3a and floating seal 3b combined. In some embodiments, a system or device disclosed herein comprises one or more additional needle body openings (e.g., a third needle body opening, 6b3) that are proximal to needle body opening 6b2, distal to needle body opening 6b1, and/or between needle body openings 6b1 and 6b2, such that a third lumen is formed and a third flowable composition may be delivered before the first flowable composition, after the second flowable composition, or between the first and second flowable compositions.

Described below are multiple embodiments to control the termination of the injection process using a medical puncturing device disclosed herein.

In some embodiments, when the pre-filled injection device or system is in a fluidic communication state, floating seal 3 moves forward due to the elastic engagement with actuation member 2 until it seals needle body opening 6b. Once needle body opening 6b is sealed, the injection process is terminated. In some embodiments, the axial position of needle body opening 6b within the flowable composition lumen 7 limits the maximum injection volume of the medical puncturing device. In some embodiments, when needle body opening 6b is blocked or sealed by floating seal 3, floating seal 3 has not contacted a wall at the syringe barrel distal closed end. In some embodiments, flowable composition lumen 7 is not completely emptied and there is still flowable composition between floating seal 3 and the wall at the syringe barrel distal closed end.

In some embodiments, when flowable composition lumen 7 needs to be emptied, floating seal 3 can be designed to seal needle body opening 6b when the floating seal contacts the syringe barrel distal closed end. In some embodiments, needle body opening 6b is at the distal end of flowable composition lumen 7. In some embodiments, floating seal 3 contacts a wall at the syringe barrel distal closed end and needle body opening 6b is blocked or sealed by floating seal 3 and/or the wall at the syringe barrel distal closed end. In some embodiments, flowable composition lumen 7 is emptied and there is no or little flowable composition between floating seal 3 and the wall at the syringe barrel distal closed end.

In some embodiments, as the flowable composition inside flowable composition lumen 7 gradually enters the apparent or potential tissue void, cavity, or vessel, there can be a state wherein the fluid pressure inside flowable composition lumen 7 reaches equilibrium with the pressure in the apparent or potential tissue void, cavity, or vessel. At this time, floating seal 3 no longer moves, due to the balance of forces. In order to continue injection and/or empty flowable composition lumen 7, additional force is needed on floating seal 3 in order to move it forward toward the syringe barrel distal closed end.

For example, as shown in FIGS. 2A-2E, one, two, or more axially extending sliding grooves (not shown) can be provided on a body wall of syringe barrel 1. A slider matching a sliding groove can be provided on actuation member 2 (e.g., a slider can comprise a portion of actuation member 2 extending outside of syringe barrel 1), thus increasing the upper limit of the movement distance or stroke of actuation member 2 since the movement is not limited by the proximal end of actuation member 2. When floating seal 3 can no longer move due to the equilibrium of forces (e.g., between pressure inside flowable composition lumen 7 and the apparent or potential tissue void, cavity, or vessel), more pressure can be applied on a slider of actuation member 2 to drive actuation member 2 forward distally, which in turn can increase the elastic resilience between floating seal 3 and actuation member 2, thus breaking the force equilibrium and moving floating seal 3 forward toward the distal end of the syringe barrel. This way, more flowable composition can be expelled from flowable composition lumen 7, in some embodiments emptying flowable composition lumen 7.

In some embodiments, other drive structures can be used to move floating seal 3 further until it contacts a wall of the syringe barrel distal closed end. Exemplary drive structures are described below.

In some embodiments, the pre-filled injection device or system comprises an element configured for an operator to manually control movement of the floating seal using one or both hands. In some embodiment, the manual control element can be moved using one or more fingers, for example, one finger of the same hand holding the syringe barrel. In some embodiments, the manual control element is fixed to floating seal 3 and partially extends outside the syringe barrel. In some embodiments, when the flowable composition volume injected into the apparent or potential tissue void, cavity, or vessel does not reach a target volume, while floating seal 3 is no longer moving due to the equilibrium of forces, the operator can drive further movement of floating seal 3 forward by moving the portion of the manual control element that extends outside the syringe barrel, until the expelled flowable composition volume reaches the target volume. In some embodiments, using the manual control element helps empty flowable composition lumen 7. These embodiments are not limited to situations where flowable composition lumen 7 needs to be emptied.

In some embodiments, the pre-filled injection device or system can achieve delivery (e.g., via injection) of a flowable composition of a defined volume with precision, and/ or the ability to control the volume to be delivered. In some embodiments, the defined volume is a preset volume prior to the delivery. In some embodiments, the defined volume is a pre-filled volume prior to the delivery. In some embodiments, the defined volume is one of multiple volumes that an operator can select during the delivery, and the delivered volume may be different from a preset volume. In some embodiments, as shown in FIGS. 1A-1E, FIGS. 2A-2E, and FIGS. 11A-11B, axial stopper 1a is provided inside the syringe lumen and distal to floating seal 3, and is used to limit the forward movement of floating seal 3. In some embodiments, when the medical puncturing device is in the fluidic communication state, needle body opening 6b can be distal to axial stopper 1a, and floating seal 3 can move forward due to the elastic engagement with actuation member 2.

In some embodiments, floating seal 3 is moved to the position limited by axial stopper 1a. In some embodiments, when floating seal 3 moves to the position limited by axial stopper 1a, pressure in flowable composition lumen 7 is still no less than the pressure inside the apparent or potential tissue void, cavity, or vessel. In some embodiments, floating seal 3 can be pushed forward to the position limited by axial stopper 1a by the elastic resilience between floating seal 3 and actuation member 2, and there is no need to rely on additional driving structure or force to move floating seal 3 to the position limited by axial stopper 1a.

In some embodiments, before floating seal 3 is moved to the position limited by axial stopper 1a by the elastic resilience between the floating seal and actuation member 2, pressure in flowable composition lumen 7 has already become equal with the pressure inside the apparent or potential tissue void, cavity, or vessel (that is, due to balance of forces, floating seal 3 is no longer moving before it reaches axial stopper 1a). At this time, just by the elastic resilience between floating seal 3 and actuation member 2, floating seal 3 is not pushed forward to the position limited by axial stopper 1a. Thus, in some embodiments, one or more additional driving structure or mechanism can be employed to further push forward floating seal 3. For example, the additional driving structure or mechanism can comprise a manual control element described herein (e.g., as shown in FIGS. 2A-2E). In some embodiments, axial stopper la provides a mechanism for achieving fluid injection of set volumes.

Described below are multiple embodiments for puncture and injection timing of a medical puncturing device disclosed herein.

In some embodiments, when the pre-filled injection device or system is in pre-puncture state, that is, when the length of hollow puncture needle 6 extending from the syringe barrel distal closed end is within the pre-puncture length range (or when hollow puncture needle 6 has already pierced the syringe barrel distal closed end but has not yet started puncturing the organism or a tissue thereof), needle body opening 6b remains above (e.g., proximal to) flowable composition lumen 7. When provided in this way, early leakage from needle distal opening 6a can be prevented and the reliability of the medical puncturing device can be improved.

In some embodiments, corresponding structure(s) can be provided on the pre-filled injection device or system to prevent early leakage before hollow puncture needle 6 punctures the tissue and/or before needle distal opening 6a reaches the apparent or potential tissue void, cavity, or vessel. For example, axially extending circular contacting element 1b (which is optional) can be formed at the syringe barrel distal closed end. In some embodiments, the axial length of circular contacting element 1b is set to be the same as the difference between the upper and lower limits of the pre-puncture length range of hollow puncture needle 6 (that is, the difference in needle pre-puncture lengths between when hollow puncture needle 6 pierces the syringe barrel distal closed end and when it starts puncturing the organism or tissue). Under this setting, as long as the distal end of hollow puncture needle 6 is still within the axial length range of circular contacting element 1b, early leakage will not happen at needle distal opening 6a. When puncturing, circular contacting element 1b can come into contact with the surface of the organism or tissue first to stabilize the medical puncturing device. Then, pressure can be applied to actuation member 2 to start the puncture operation.

In some embodiments, when the pre-filled injection device or system is in the surface tissue puncture state, that is, when the length of hollow puncture needle 6 extending from the syringe barrel distal closed end is within the surface tissue puncture length range (or when the distal end of hollow puncture needle 6 has pierced the surface tissue but has not yet entered the apparent or potential tissue void, cavity, or vessel), needle body opening 6b is at least partially connected to flowable composition lumen 7. In some embodiments, before the distal end of hollow puncture needle 6 pierces into the apparent or potential tissue void, cavity, or vessel, fluidic communication among flowable composition lumen 7, needle distal opening 6a and needle body opening 6b is established. In some embodiments, the flowable composition in lumen 7 can enter the needle body passageway (via needle body opening 6b) of hollow puncture needle 6 in advance, removing at least part of the air that may be in the needle body passageway, thereby reducing the amount of air entering the apparent or potential tissue void, cavity, or vessel.

In some embodiments, when the distal end of hollow puncture needle 6 starts to pierce into the surface tissue, needle body opening 6b starts to connect with flowable composition lumen 7. In some embodiments, when the distal end of hollow puncture needle 6 pierces into the apparent or potential tissue void, cavity, or vessel, the needle body passageway of hollow puncture needle 6 has already been filled with the flowable composition, thereby eliminating or reducing the possibility of air entering the apparent or potential tissue void, cavity, or vessel.

In some embodiments, when the pre-filled injection device or system is in the fluidic communication state, that is, when the length of hollow puncture needle 6 extending from the syringe barrel distal closed end is within the fluidic communication length range (or when the distal end of hollow puncture needle 6 has pierced into the apparent or potential tissue void, cavity, or vessel), needle body opening 6b has been positioned inside flowable composition lumen 7, achieving maximum flow at needle body opening 6b and thereby increasing injection speed.

The embodiments described herein can be implemented separately or in any suitable combination.

In some embodiments, a device disclosed herein can prevent fluid backflow and/or reverse spill through needle body opening 6b.

In some embodiments, there is a risk for fluid backflow and/or reverse spill from needle body opening 6b when needle distal opening 6a is connected with flowable composition lumen 7, while needle body opening 6b is still at the proximal end of floating seal 3. In some embodiments, there is a risk for fluid backflow and/or reverse spill from needle body opening 6b when needle distal opening 6a is inside the apparent or potential tissue void, cavity, or vessel, while needle body opening 6b is still at the proximal end of floating seal 3. In some embodiments, an elastic sheath 4 covering the outside of hollow puncture needle 6 can be provided within the actuation unit (e.g., elastic movement unit), e.g., between the needle base and floating seal 3. In some embodiments, when needle body opening 6b is at the proximal end of floating seal 3 (e.g., when needle body opening 6b is not connected to flowable composition lumen 7), elastic sheath 4 can keep the needle body opening 6b sealed, thereby effectively avoiding backflow and/or reverse spill of the flowable composition, preventing contamination of the area proximal to floating seal 3, reducing fluid loss, and improving product reliability.

In some embodiments, elastic sheath 4 is not used to seal needle body opening 6b, but simply as an elastic engagement part between floating seal 3 and actuation member 2. In some embodiments, by moving actuation member 2 forward, elastic sheath 4 between floating seal 3 and actuation member 2 can become compressed, thereby forming elastic resilience between floating seal 3 and actuation member 2, which can in turn drive floating seal 3 forward. In some embodiments, the elastic engagement part between floating seal 3 and actuation member 2 can comprise or be a spring 5, which is attached to floating seal 3 and actuation member 2 at its two axial ends, respectively. The attachment at either or both ends of the spring can be direct or indirect. The attachment at either or both ends of the spring can be releasable or not releasable. The spring, the floating seal, and the actuation member (e.g., pressing element) can be separately manufactured and then assembled in any suitable order. Alternatively, any two or more of the spring, the floating seal, and the actuation member (e.g., pressing element) can be integral, e.g., made as one piece. Spring 5 and elastic sheath 4 can be implemented separately or in combination.

In some embodiments, the elastic engagement between floating seal 3 and actuation member 2 can be achieved through other methods besides providing one or more elastic engagement parts. For example, floating seal 3 and actuation member 2 can be provided as a one-piece integrated actuation unit (e.g., elastic movement unit).

In some embodiments, provided herein are devices and methods for implantation into apparent or potential tissue gaps, cavity systems, and vessels using a medical puncturing device disclosed herein. For ease of understanding, a stent is used as an example for the implanted medical device. In some embodiments, a method disclosed herein comprises using a stent guiding structure for guiding stent 11 into the needle body passageway of hollow puncture needle 6. In some embodiments, a stent guiding structure is provided in a pre-filled injection device or system disclosed herein.

In some embodiments, as shown in FIGS. 6-8, the stent guiding structure comprises an angled guiding groove 3a, which is provided in or engages floating seal 3 and extends towards hollow puncture needle 6 at an angle. In some embodiments, when flowable composition lumen 7, needle body opening 6b, and needle distal opening 6a are connected, a flowable composition can enter and expand the apparent or potential tissue void, cavity, or vessel. In some embodiments, stent 11 can be implanted through angled guiding groove 3a, needle body opening 6b, the needle body passageway of hollow puncture needle 6, and needle distal opening 6a into the expanded apparent or potential tissue void, cavity, or vessel.

It should be noted that, angled guiding groove 3a can be provided as a groove through floating seal 3 in a proximal/distal direction, or as a non-through groove formed on a proximal surface of floating seal 3.

In some embodiments, angled guiding groove 3a is a through groove. In some embodiments, the catheter guiding structure further comprises valve 9 provided in or engages angled guiding groove 3a, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into needle body opening 6b through the opened valve. In some embodiments, the catheter guiding structure further comprises a guiding groove plug configured to be removably inserted in angled guiding groove 3a, and the guiding groove plug can be pulled out when catheter 11 needs to be implanted.

In some embodiments, angled guiding groove 3a is a non-through groove. In some embodiments, the angled guiding groove is punctured directly by catheter 11 to be implanted. In some embodiments, the angled guiding groove is punctured by a piercing component other than the catheter, and catheter 11 can be threaded through the punctured opening into needle body opening 6b.

In some embodiments, to match the guiding direction of angled guiding groove 3a, needle body opening 6b can be provided as an angled opening, which opens obliquely backwards, so that needle body opening 6b can align with angled guiding groove 3a, thereby precisely guiding catheter 11 through the angled guiding groove and into the needle body opening.

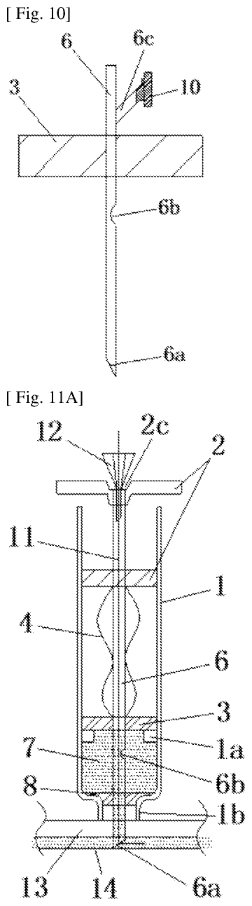

In some embodiments, for example as shown in FIG. 9 and FIG. 10, the catheter guiding structure comprises an angled guiding needle hole 6c which is formed or provided on the body wall of hollow puncture needle 6 and opens obliquely backwards. In some embodiments, angled guiding needle hole 6c remains proximal to floating seal 3, for example, when the medical puncturing device is in a fluidic communication state. In some embodiments, catheter 11 can be threaded into the needle body passageway of hollow puncture needle 6 through angled guiding needle hole 6c. In some embodiments, catheter 11 can be implanted into an apparent or potential tissue void, cavity, or vessel (or an apparent or potential tissue void, cavity, or vessel that has been expanded with a flowable composition) through needle distal opening 6a.

In some embodiments, the catheter guiding structure can further comprise valve 9 provided in or engages angled guiding needle hole 6c, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into a needle body passageway (which may be connected to or separate from the needle body passageway connecting needle body opening 6b and needle distal opening 6a) through the opened valve and angled guiding needle hole 6c. In some embodiments, the catheter guiding structure can further comprise needle hole plug 10 configured to be removably inserted in angled guiding needle hole 6c, and needle hole plug 10 can be pulled out for the implantation operation of catheter 11 to begin. In some embodiments, guiding needle hole 6c is connected needle distal opening 6a. The needle body passageway connecting needle distal opening 6a and needle body opening 6b can be the same as or separate from the needle body passageway connecting needle distal opening 6a and guiding needle hole 6c. In some embodiments, guiding needle hole 6c is connected to a needle distal opening other than needle distal opening 6a connected to needle body opening 6b. The needle body passageway connecting needle body opening 6b to a needle distal end can be completely separate from the needle body passageway connecting guiding needle hole 6c to a needle distal end. The needle body passageway connecting needle body opening 6b to a needle distal end can be at least partially overlapping or in fluidic communication with the needle body passageway connecting guiding needle hole 6c to a needle distal end.

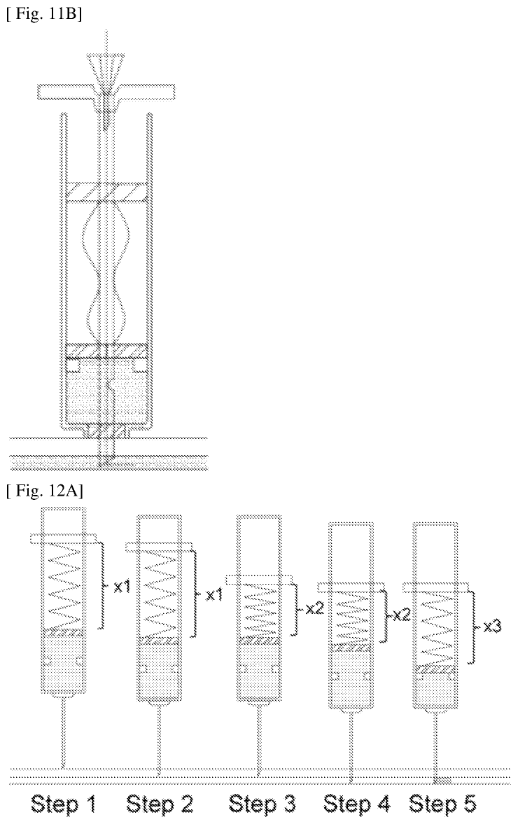

In some embodiments, for example as shown in FIGS. 11A-11B, the catheter guiding structure comprises a central guiding groove 2c that is formed or provided on a proximal surface of actuation member 2. In some embodiments, central guiding groove 2c comprises an aperture or can form an aperture in the center of proximal surface of actuation member 2. In some embodiments, central guiding groove 2c can be punctured to provide an aperture. In some embodiments, a needle proximal opening is provided on hollow puncture needle 6 and is aligned with central guiding groove 2c along the axis. In some embodiments, when catheter 11 needs to be implanted, central guiding groove 2c can be punctured and catheter 11 can be threaded into a needle body passageway (which may be connected to or separate from the needle body passageway connecting needle body opening 6b and needle distal opening 6a) through the punctured opening of central guiding groove 2c and the needle proximal opening of hollow puncture needle 6. In some embodiments, catheter 11 can be implanted into an apparent or potential tissue void, cavity, or vessel (or an apparent or potential tissue void, cavity, or vessel that has been expanded with a flowable composition) through a needle distal opening, such as needle distal opening 6a or a different needle distal opening.

In some embodiments, disclosed herein is a kit comprising components configured to be assembled to form a pre-filled injection device or system disclosed herein.

In some embodiments, the kit for assembling a pre-filled injection device or systems comprises a puncture control module and a pre-filled flowable composition storage module (e.g., a fluid storage module). In some embodiments, the puncture control module and the flowable composition storage module are independently manufactured and/or provided. In some embodiments, the puncture control module comprises a first syringe unit, as well as an actuation unit (e.g., elastic movement unit), and hollow puncture needle 6, which are provided inside a syringe barrel of the first syringe unit. It can be seen based on the embodiments disclosed herein that the puncture control module can further comprise other parts or components, such as elastic sheath 4 and spring 5. In some embodiments, the pre-filled fluid storage module comprises a second syringe unit, flowable composition lumen 7 which is formed inside a syringe barrel of the second syringe unit, and a module packaging component which is removably provided at the proximal end of the second syringe unit. In some embodiments, a removable connection structure is formed between the first syringe unit and the second syringe unit. In some embodiments, the first syringe unit and the second syringe unit form syringe barrel 1 after being connected with each other. It can be seen based on the embodiments disclosed herein that the fluid storage module can further comprise other parts such as distal seal 8.

In some embodiments, the puncture control module and the fluid storage module can be manufactured, assembled, and/or packaged separately, and then assembled with each other and optionally with other modules, components, and/or parts into the medical puncturing device disclosed herein. In some embodiments, the module packaging component is used to seal the proximal end of flowable composition lumen 7. In some embodiments, when assembling the puncture control module and the fluid storage module, the module packaging component can be removed.

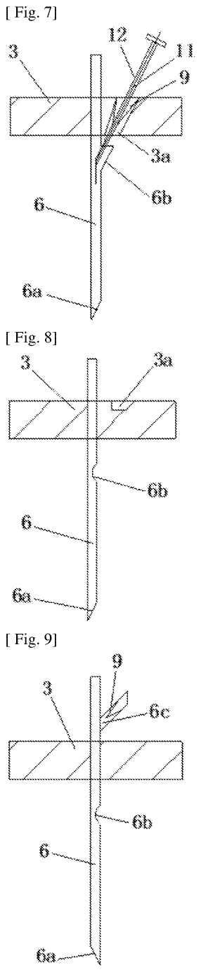

In some embodiments, provided herein is a pre-filled medical apparatus assembly and a system comprising the same. As shown in FIG. 7 and FIGS. 11A-11B, in some embodiments the medical apparatus assembly comprises stent 11 and the medical puncturing device comprising the stent guiding structure disclosed herein. In some embodiments, stent 11 can be implanted into an apparent or potential tissue void, cavity, or vessel by the pre-filled injection device or system. The medical apparatus assembly described herein can have all of the technical effects provided by the pre-filled injection device or system.

In some embodiments, the medical apparatus assembly comprises hollow auxiliary guiding needle 12, which is matched to be used with the stent guiding structure. In some embodiments, the needle body passageway diameter of auxiliary guiding needle 12 is large enough to accommodate stent 11 and allow the stent to thread in. In some embodiments, during an operation to implant stent 11, auxiliary guiding needle 12 is connected to the stent guiding structure so that stent 11 can sequentially go through the needle body passageway of auxiliary guiding needle 12, the stent guiding structure, the needle body passageway of hollow puncture needle 6, and then into an apparent or potential tissue void, cavity, or vessel through needle distal opening 6a. In some embodiment, the apparent or potential tissue void, cavity, or vessel is expanded with a flowable composition using a pre-filled injection device or system disclosed herein, prior to the implant of the stent. In some embodiment, the stent is implanted as the apparent or potential tissue void, cavity, or vessel is being expanded with a pre-filled flowable composition using a pre-filled injection device or system herein. In some embodiment, the stent is implanted prior to the apparent or potential tissue void, cavity, or vessel being expanded with a flowable composition using a pre-filled injection device or system disclosed herein.

In some embodiments, as shown in FIG. 7, the stent guiding structure comprises through angled guiding groove 3a and one-way valve 9, which is embedded in angled guiding groove 3a and can be opened and closed. In some embodiments, needle body opening 6b is provided as an angled opening which opens obliquely backwards. In some embodiments, when implanting stent 11, auxiliary guiding needle 12 is used to open one-way valve 9 so that the auxiliary guiding needle can be positioned inside angled guiding groove 3a. In some embodiments, the distal end of auxiliary guiding needle 12 advances into needle body opening 6b, and stent 11 can sequentially advance through the needle body passageway of auxiliary guiding needle 12, the needle body passageway of hollow puncture needle 6, and the needle distal opening 6a and then be implanted into an apparent or potential tissue void, cavity, or vessel.

In some embodiments, as shown in FIGS. 11A-11B, the stent guiding structure comprises a central guiding groove 2c. In some embodiments, a needle proximal opening is formed on hollow puncture needle 6, which is aligned with central guiding groove 2c along its axis. In some embodiments, when implanting stent 11, central guiding groove 2c can be punctured by auxiliary guiding needle 12, such that auxiliary guiding needle 12 is axially aligned with the proximal opening of hollow puncture needle 6. In some embodiments, stent 11 is threaded into a needle body passageway of hollow puncture needle 6 by sequentially advancing through a needle body passageway of auxiliary guiding needle 12, and a proximal opening of hollow puncture needle 6, and is then implanted into an apparent or potential tissue void, cavity, or vessel through a needle distal opening such as needle distal opening 6a.

In some embodiments, disclosed herein is a pre-filled system, comprising: a syringe barrel comprising a proximal end and a distal end; a floating seal in the syringe barrel; a needle base proximal to the floating seal, a piston rod between the floating seal and the needle base, the needle base and the piston rod elastically engaging each other; and a needle in the piston rod, the needle comprising a needle proximal end engaging the needle base and a needle distal end, wherein the needle comprises: (i) a needle distal opening, (ii) a needle body opening between the needle proximal end and the needle distal end, wherein the needle body opening is proximal to the needle distal opening, and (iii) a needle body passageway connecting the needle distal opening and the needle body opening, wherein the needle base is configured to advance the needle distally through the piston rod and toward and/or through the floating seal.

In some embodiments, the floating seal can be fixedly attached to the distal end of the piston rod and form a sliding and sealing engagement with an inner surface of the syringe barrel. In any of the embodiments herein, the needle base can fixedly engage an actuation member (e.g., pressing element), and a spring can engage the actuation member and the piston rod, thereby providing the elastic engagement between the needle base and the piston rod.

In some embodiments, advancement of the needle distally through the piston rod and through the floating seal can occur without moving the floating seal distally, when the needle distal opening is in a tissue or an apparent or potential tissue void, cavity, or vessel providing a higher pressure at the needle distal opening than the pressure at the needle body opening. In some embodiments, the tissue resistance or tissue pressure does not allow injection of the flowable composition through the needle distal opening into the tissue, and the floating seal (as well as the piston rod in embodiments that have one) is not moved distally under a force from the spring, even though the needle can be advanced distally under a force from the pressing shaft. For instance, when the tissue pressure does not allow injection, a needle distal opening of the needle can be in the tissue while a needle body opening is distal to the floating seal and contacting the flowable composition. The floating seal can maintain its position in an axial direction while the needle is further advanced until the needle distal opening reaches an apparent or potential tissue void, cavity, or vessel.

In some embodiments, the floating seal can be moved distally, when the needle distal opening is in a tissue or an apparent or potential tissue void, cavity, or vessel providing a lower pressure at the needle distal opening than the pressure at the needle body opening. In some embodiments, the tissue resistance or tissue pressure allows injection of the flowable composition through the needle distal opening into the tissue, and the floating seal (as well as the piston rod in embodiments that have one) is moved distally under a force from the spring, and the needle does not need to be advanced distally. For instance, when the tissue pressure allows injection, a needle distal opening of the needle can be in the apparent or potential tissue void, cavity, or vessel, while a needle body opening is distal to the floating seal and contacting the flowable composition. The floating seal can be moved distally and the flowable composition is discharged from the needle distal opening while the needle is not further advanced distally.

In some embodiments, provided herein is a method of using a pre-filled injection device or system described herein for medical penetration. In some embodiments, a preassembled pre-filled injection device or system is provided, as shown in FIG. 17A. In some embodiments, the housing of the preassembled device can be rotated to separate the syringe from the main body of the device. In some embodiments, a proximal portion of the syringe can be in threaded engagement with a distal portion of the housing. For example, the proximal portion of the syringe can comprise threaded grooves on its internal surface which are configured to engage threaded ridges on the outside surface of the distal portion of the housing, as shown in FIG. 17B.

After separation of the syringe, in some embodiments, the proximal end of the piston rod is exposed. A handle can be attached to the piston rod, e.g., via threaded engagement with the proximal end of the piston rod, as shown in FIG. 17C. In some embodiments, an adapter comprising an adapter needle enclosed therein can be attached to the syringe. In some embodiments, the adapter comprises a distal opening and a proximal opening. In some embodiments, the distal end of the syringe (e.g., with the distal seal attached thereto) is inserted into the proximal opening of the adapter, thereby contacting the adapter needle with the distal seal attached to the syringe. In some embodiments, the proximal end of the adapter needle passes through the distal seal attached to the syringe, such that a proximal opening of the adapter needle is inside the internal lumen of the syringe. In some embodiments, a container or a portion thereof containing a pre-filled flowable composition (e.g., a drug composition) is inserted into the distal opening of the adapter, thereby contacting the adapter needle with the container. In some embodiments, the distal end of the adapter needle inserts into the container, such that a distal opening of the adapter needle is inside the container and capable of establishing a fluid communication between the flowable composition and the internal lumen of the syringe. In some embodiments, the handle is pulled to move the piston rod proximally and draw the flowable composition into the internal lumen of the syringe through the adapter needle, and undesired gas can be expelled by pushing the handle to move the piston rod distally. By pulling and/or pushing the handle, the seal at the distal end of the piston rod and inside the syringe can be placed at a position to set a suitable volume of the flowable composition in the syringe, for example, 0.1 mL or 0.05 mL, as shown in FIG. 17D, and the handle and the adaptor can then be disconnected from the piston rod and the syringe, respectively. The syringe with the flowable composition inside can be connected with the body of the device, e.g., by inserting the syringe needle (e.g., 6 as shown in FIG. 17B) into the piston rod (e.g., 15 as shown in FIG. 17D), inserting the piston rod into the guide tube inside the housing, and screwing the proximal end of the syringe back onto the distal end of the housing, as shown in FIG. 17E. In some embodiments, the control knob can be rotated to advance the pressing shaft in a distal direction, thereby advancing the syringe needle attached to the pressing shaft distally toward and/or through the seal inside the syringe. The syringe needle can be further advanced to pass through the sealing tip, as shown in FIG. 17F, and to pierce into the sclera of the eye. In some embodiments, since the sclera is a dense tissue, the pressure at the distal opening of the syringe needle is greater than the pressure at the body opening of the syringe needle, which can be in fluid communication with the flowable composition inside the syringe; under such conditions, the syringe needle may be further advanced in the sclera without changing the position of the floating seal inside the syringe. In some embodiments, the position of the floating seal inside the syringe is monitored as an operator pushes the pressing shaft to advance the syringe needle. Once the distal opening of the syringe needle is outside the sclera and into the choroid/ciliary body, the pressure at the distal opening of the syringe needle decreases, and the pressure at the body opening of the syringe needle can drive the flowable composition through the needle body passageway and discharge it from the syringe needle distal opening, thereby creating and expanding a suprachoroidal space containing the flowable composition. Since a portion of the flowable composition inside the syringe is discharge, the seal (along with the piston rod) is moved to a more distal position in the syringe. Thus, by observing the movement of the seal, an operator can determine whether the distal opening of the syringe needle has exited a first tissue and reached a second, less dense tissue, e.g., from the sclera into the choroid/ciliary body. In some embodiments, as soon as the seal moves and passes a preset mark or indicator line for volume (e.g., 0.1 mL or 0.05 mL), the advancement of the syringe needle distally is stopped.

In some examples, for instance as shown in FIGS. 17A-17F, part of the fluid lumen in the pre-filled injection device or system is not prefilled with a flowable material or composition, and the flowable material or composition is drawn from a container into the syringe prior to delivery into a tissue or apparent or potential tissue void, cavity, or vessel.

In some examples, the pre-filled injection device or system disclosed herein is prefilled with a flowable material or composition. In some embodiments, the syringe (e.g., syringe 1 show in FIG. 16) can be provided in one or more parts. In some embodiments, a container (e.g., a syringe unit) can comprise a cylindrical wall sealingly engaging a fixed seal (which can be fixed to the container at a distal end of the container and can be passed through by the needle) and a floating seal (which can move inside the container and can be passed through by the needle), and the space enclosed by the cylindrical wall, the fixed seal and the floating seal can be prefilled with a flowable material or composition. In some embodiments, the pre-filled injection device or system can comprise a first syringe unit and the container can be a second syringe unit configured to engage the distal end of the first syringe unit. The container (e.g., syringe unit) can be inserted into or attached to the body (e.g., to the first syringe unit) of the device prior to or after the flowable material or composition is filled into the container (e.g., syringe unit). In some embodiments, the floating seal in the container (e.g., syringe unit) may contact the distal end of the piston rod, thereby establishing an engagement between the piston rod and the floating seal that transmits a force from the spring to the floating seal. The fixed seal at the distal end of the container (e.g., syringe unit) may contact a contacting element at the distal end of the device, and the contacting element can be a distal seal of the syringe. In some embodiments, the fixed seal of the container (e.g., syringe unit) also serves as a distal seal of the syringe and/or as a contacting element. In some embodiments, the container (e.g., syringe unit) can be configured to at least partially insert into a syringe barrel. In some embodiments, the fixed seal sealingly engages the container (e.g., syringe unit) which in turn engages an inside wall of the syringe barrel. In some embodiments, the fixed seal sealingly engages both the container (e.g., syringe unit) and an inside wall of the syringe barrel. The engagement between the container (e.g., syringe unit) and the syringe barrel and the engagement between the fixed seal and a wall of the container can comprise any suitable engagement, such as via insertion, a threaded engagement, a non-threaded engagement, engagement secured by a clip, engagement secured by a gland, or any combination thereof.

### VI. Methods and Devices for Drainage from the Eye

Glaucoma is the leading cause of irreversible blindness. Current treatments use drugs or surgery to reduce intraocular pressure (IOP). Drug-free approaches have been used (see, e.g., Chae et al., Adv. Sci. 2021, 8, 2001908) but the drug-free, nonsurgical method only lowers IOP for about 4 months.

In some embodiments, disclosed herein are methods, compositions and devices for reducing intraocular pressure, for instance for use in treating glaucoma, in a subject in need thereof. In some embodiments, the methods and uses of the compositions and devices may comprise expanding the suprachoroidal space (SCS) of an eye with one or more viscoelastic agents. In some embodiments, the SCS is expanded with an in situ-forming depot (which may contain one or more drugs or can be drug-free) in the SCS. In some embodiments, the SCS is expanded with an in situ-forming hydrogel, for instance, one is injected in the SCS using a microneedle. In some embodiments, the SCS is expanded with one or more viscoelastic agents configured to form a permanent or semi-permanent structure in the SCS, thereby providing prolonged expansion of the SCS. In some embodiments, the SCS is expanded with one or more viscoelastic agents, followed by implanting a permanent or semi-permanent structure in the SCS, thereby providing prolonged expansion of the SCS. In some embodiments, the SCS is expanded with a composition comprising hyaluronic acid (HA) hydrogel. In some embodiments, the SCS is expanded (e.g., compared to its natural state of being a potential tissue void) for at least or about four months, at least or about six months, at least or about eight months, at least or about one year, at least or about two years, at least or about three years, at least or about four years, at least or about eight years, at least or about twelve years, or longer.

Aqueous humor flows out of the eye primarily through the conventional outflow pathway that includes the trabecular meshwork and Schlemm's canal. However, a fraction of aqueous humor passes through an alternative or ‘unconventional’ route that includes the ciliary muscle, supraciliary and suprachoroidal spaces. In some embodiments, SCS expansion increases the drainage of aqueous humor from the eye via the unconventional pathway, which thereby lowers intraocular pressure (IOP). In some embodiments, IOP reduction is correlated with SCS expansion. In some embodiments, there is no difference in pressure-dependent outflow by the conventional pathway between eyes with expanded SCS and untreated eyes. In some embodiments, the methods, compositions and devices provided herein enable extended IOP reduction for treating ocular hypertension and/or glaucoma without the need of drugs or surgery.

Any of the systems and devices disclosed herein can be used for placing a permanent or semi-permanent structure such as a stent into an eye for lowering intraocular pressure, e.g., for treating glaucoma, comprising: (a) inserting a needle into the eye at an injection site for injection into a suprachoroidal space (SCS) in the eye; (b) delivering a composition (e.g., a viscoelastic composition) through the needle to form the SCS; and (c) positioning a stent (e.g., a micro stent) in the SCS, thereby placing the stent in the eye to sustain the SCS in an expanded state and facilitate drainage of aqueous humor.

In some embodiments, a method disclosed herein comprises: (a) inserting a needle into the eye to form a delivery passageway in the eye, wherein the delivery passageway ends in a region between the sclera and the choroid in the eye; (b) delivering a composition (e.g., a viscoelastic composition) through the needle to form an SCS; (c) positioning a stent (e.g., a micro stent) in the expanded SCS, wherein the stent is releasably coupled to the needle; and (d) releasing the needle from the stent, thereby placing the stent in the eye to sustain the SCS and facilitate drainage of fluid (e.g., from the anterior chamber) through the SCS. Before releasing the stent from the needle, the stent can be at least partially inside the needle and/or at least partially outside the needle (e.g., in the form of a hollow tube through which a portion of the needle can pass).

In some embodiments, a method disclosed herein comprises: (a) inserting a needle into the eye at an injection site for injection into a suprachoroidal space (SCS) in the eye; (b) delivering a composition (e.g., a viscoelastic composition) through the needle to form the SCS; and (c) through the injection site or an expanded insertion site (e.g., formed by expanding the injection site, for instance, by surgery), positioning a stent (e.g., a micro stent) in the SCS, thereby placing the stent in the eye to sustain the SCS in an expanded state and facilitate drainage of aqueous humor. The stent can be inserted through the injection site or the expanded insertion site to further expand the SCS formed by the injection of the viscoelastic composition, and the presence of the viscoelastic composition during the insertion of the stent can facilitate dissection between the sclera and the choroid, provide lubrication of the tip of the stent as it moves between the sclera and the choroid (e.g., on a plane that is parallel to the equator of the eye ball), and minimize tissue damage during the implanting process.

FIG. 18A shows an exemplary ab externo method, where an viscoelastic agent is injected between the sclera and the choroid, forming the SCS, followed by implanting a permanent or semi-permanent structure (e.g., a stent) to keep the SCS in an expanded state for a prolonged period of time. The implant can form a ring or a partial ring on a plane that is parallel to the equator of the eye ball, as shown in FIG. 18B. Compared to an ab interno method for drainage of aqueous humor, the ab externo method does not depend on the insertion of a stent or shunt into a muscle tissue in the eye, causes less tissue damage, is less likely to cause scar formation, and does not depend on the insertion of a stent or shunt into the anterior chamber (e.g., through piercing the anterior chamber angle).

In some embodiments, a stent disclosed herein can comprise any suitable material. Materials for manufacturing a stent include but are not limited to medical stainless steel, titanium or titanium alloy, nickel titanium alloy, TPU (thermoplastic polyurethane), e-PTFE (expanded polytetrafluoroethylene), silica gel, hydrogel, PES (polyethersulfone), SIBS (Poly (Styrene-block-IsoButylene-block-Styrene), or any combination thereof. In some embodiments, a material for the stent has a high biocompatibility, matches the mechanical properties with the eye tissue, and does not damage the eye tissue or cause adverse reaction. In some embodiments, a stent disclosed herein can but does not need have a coating. In some embodiments, a stent disclosed herein can but does not need to be coated with drugs. In some embodiments, a stent disclosed herein can contain one or more drug compositions.

In some embodiments, a stent disclosed herein can be of any suitable shape. In some embodiments, the stent is a circular tube. In some embodiments, the stent comprises a single lumen. In some embodiments, the stent comprises multiple lumens, e.g., lumens that parallel each other, each extending from one end of the stent to the other end. In some embodiments, the single or multiple lumens in the stent can contain one or more drug compositions. In some embodiments, the shape of a cross-section of the stent is circular, oval, square, or any other suitable shape. In some embodiments, any one or more of the surface(s) of the stent can be a flat surface or a curved surface.

In some embodiments, a stent disclosed herein a maker ring and/or a retention ring around the stent. In some embodiments, an annular ring around the stent, such as the retention ring, can be configured to prevent migration of the stent. The shape of the retention ring can include and is not limited to annular, barbed, fin-shaped, or any combination thereof. The structure and dimension of the stent, including structure and dimension of the marker ring and the retention ring, can be designed to match the ocular tissue structure, thereby effectively draining aqueous humor and reducing intraocular pressure without causing tissue damage and scarring.

In some embodiments, a stent disclosed herein is about 1.5 mm to about 12 mm in length, for example, about 3 mm, about 4 mm, about 5 mm, or about 6 mm in length. In some embodiments, a stent disclosed herein is about 0.1 mm to about 1 mm in diameter, for example, about 0.25 mm, about 0.3 mm, about 0.35 mm, about 0.4 mm, about 0.45 mm, or about 0.5 mm in outer diameter. In some embodiments, a stent disclosed herein is about 0.025 mm to about 0.25 mm in inner diameter, for example, about 0.05 mm, about 0.08 mm, about 0.1 mm, about 0.12 mm, or about 0.15 mm in diameter. In some embodiments, a marker ring is positioned at about 0.25 mm to about 2.5 mm from one end of the stent to allow for accurate positioning of the stent, e.g., for positioning in the SCS.

In some embodiments, a stent disclosed herein can comprise a solid structure, a porous structure, a multi-layer composite structure, a membrane stent structure, or any combination thereof. A solid structure (e.g., a uniform solid structure) is simple and effective and can establish a framework to sustain SCS expanded by viscoelastic agents. In some embodiments, to avoid or reduce the risk of fibrosis and scarring, a micro-porous material can be used to promote bio-integration of surrounding tissues into the material, which can reduce fibrosis and scarring after implant. In some embodiments, the pore size can be less than 20 microns to prevent excessive growth of tissue or cells into the pore while allowing water to freely pass through the pore. In some embodiments, an inner core or inner layer of a multi-layer composite structure can be designed to provide radial support. An outer layer of the multi-layer composite structure can be a porous or fabric layer with pore size less than 20 microns to prevent excessive growth of tissue or cells into the pore. In some embodiments, the stent is a hollow support, which can provide sufficient supporting force and flexibility.

In some embodiments, provide herein is a method of using a device disclosed herein to deliver a stent disclosed herein. In some embodiments, the stent is preloaded in the needle of a delivery system (e.g., a suprachoroidal injection syringe) and implanted into the suprachoroidal space using an ab externo approach described herein. After the tip of the needle of the delivery system punctures the sclera and reaches a choroid/ciliary body layer, a viscoelastic agent is automatically injected to open a suprachoroidal space, and the stent in the needle is pushed out to the target position, e.g., through a metal wire. The delivery system can be withdrawn to complete the implantation of the stent. A method disclosed herein can be used for minimally invasive glaucoma surgery (MIGS). In some embodiments, stent can be inserted in the needle (or can be pre-inserted in the needle prior to needle insertion and injection of viscoelastic material) and deployed at the distal end of the needle.

In some embodiments, provide herein is a method of using a device disclosed herein to deliver a stent disclosed herein. In some embodiments, a flowable material or composition (e.g., viscoelastic material) is first injected into the suprachoroidal space and the suprachoroidal space is formed. After this, the syringe needle can be removed from the injection site, leaving the suprachoroidal space filled with the viscoelastic material. In some embodiments, the injection site and path formed by the needle is further expanded to create a larger incision from the injection site, and/or a larger path from the injection site to the suprachoroidal space, and a linear member such as a cannula can be inserted, wherein a stent is releasably coupled to the linear member. In some embodiments, the stent can be a linear member that is inserted through the larger incision from the injection site. In some embodiments, the injection site is further expanded to create a larger incision from the injection site, and/or a larger path from the injection site to the suprachoroidal space, and a stent can be inserted into the suprachoroidal space. In some embodiments, the injection site and path does not need to be further expanded, and a linear member such as a cannula can be inserted, wherein a stent is releasably coupled to the linear member. In some embodiments, the injection site and path does not need to be further expanded, and a stent can be inserted.

In some embodiments, the flowable composition such as an viscoelastic composition provides lubrication of a stent or the linear member releasably coupled to a stent such that the stent or the linear member can slide along the boundary between the sclera and the choroid/ciliary body, reducing the resistance during stent insertion and/or reducing the risk of choroidal perforation or the risk of stent and/or linear member piercing into the vitreous, ciliary body, or other tissues. In some embodiments, the viscoelastic composition forms a protective layer around the stent or the linear member releasably coupled to a stent, and the protective layer can provide lubrication and guide the direction of stent insertion.

In some embodiments, provided herein is a minimally invasive method for placing the stent into the eye using a needle, without the need to surgically cut open an entire layer of the sclera, or surgically separate the sclera and the choroid/ciliary body, or sewing the cut sclera or conjunctiva after the surgery. Thus, a method disclosed herein can reduce tissue invasion, lower requirements for surgical techniques, and reduce operation time.

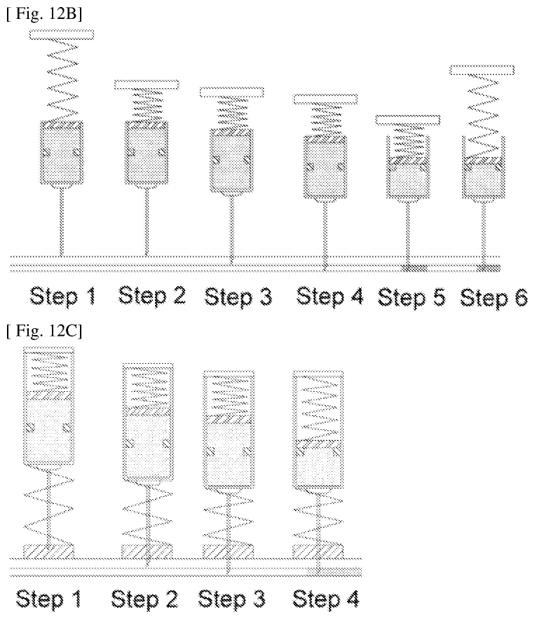

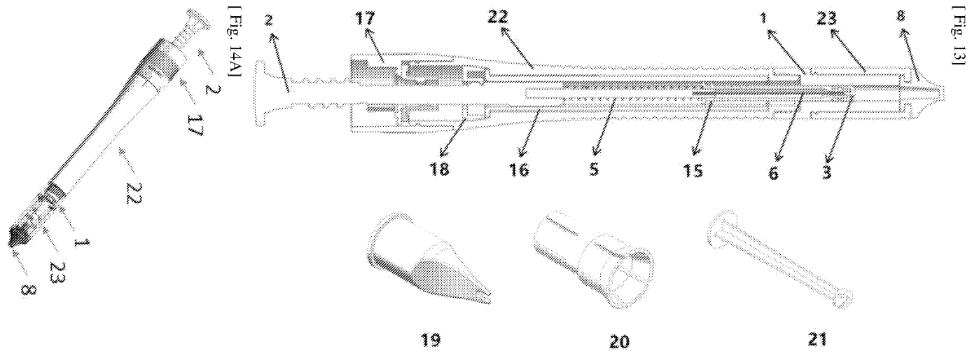

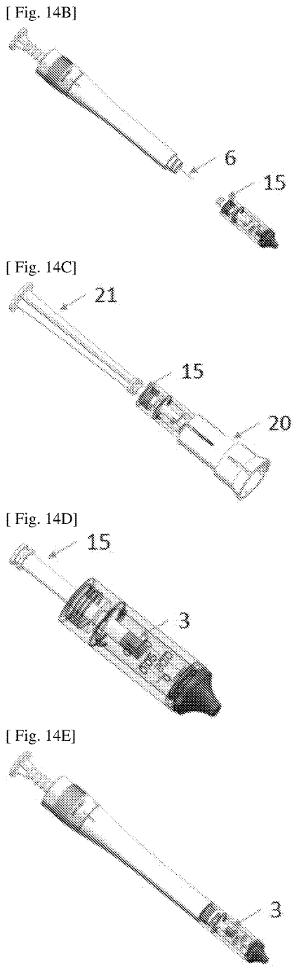

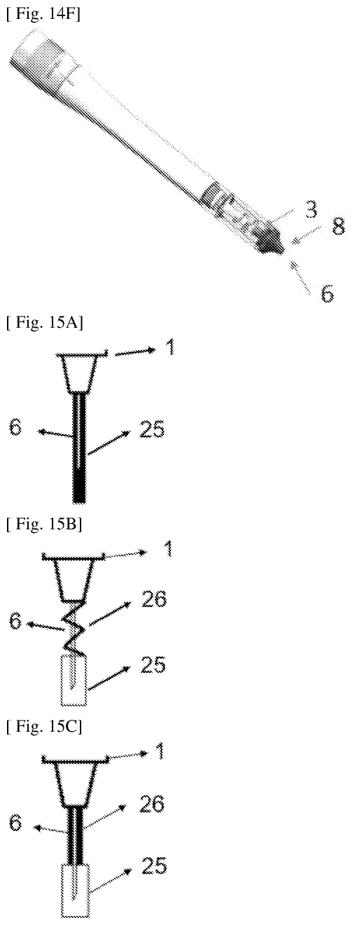

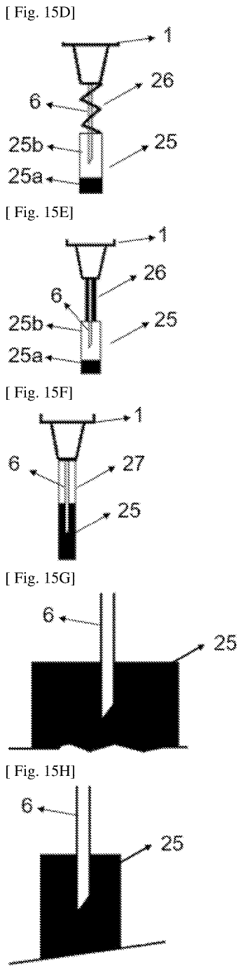

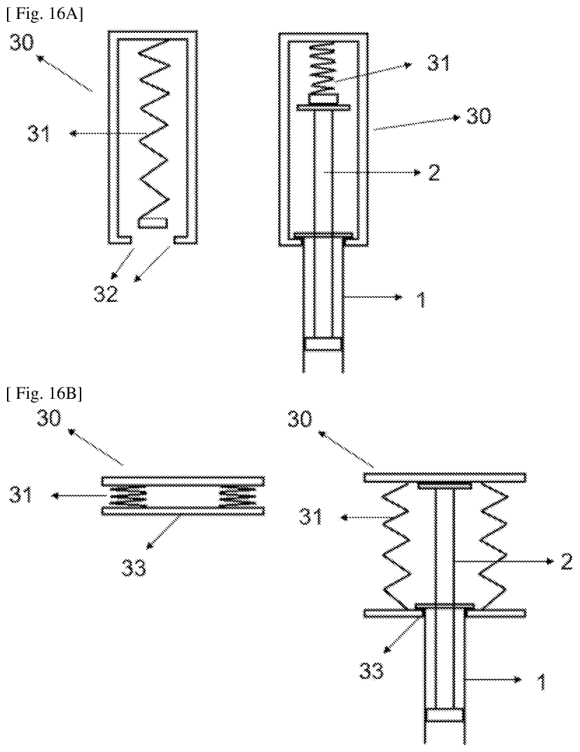

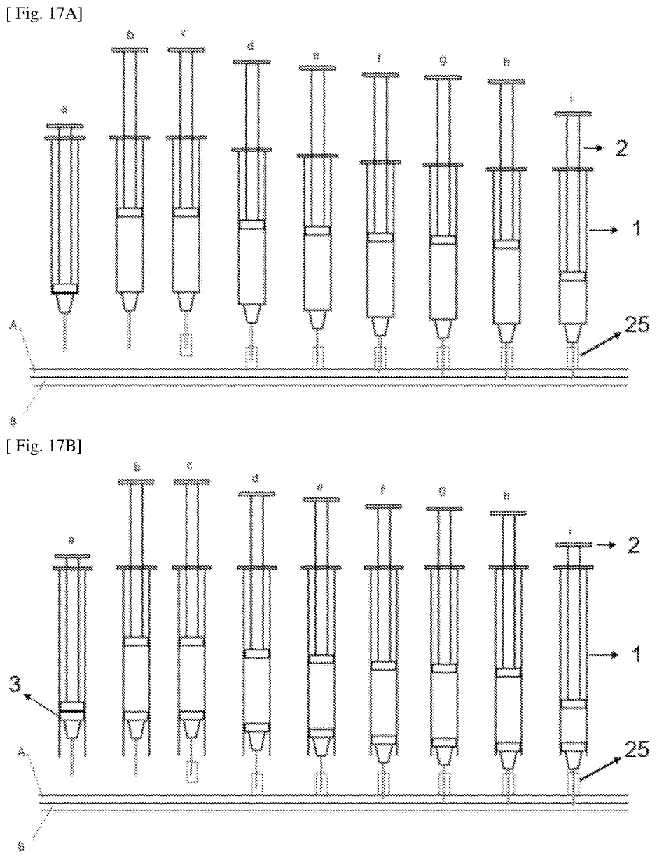

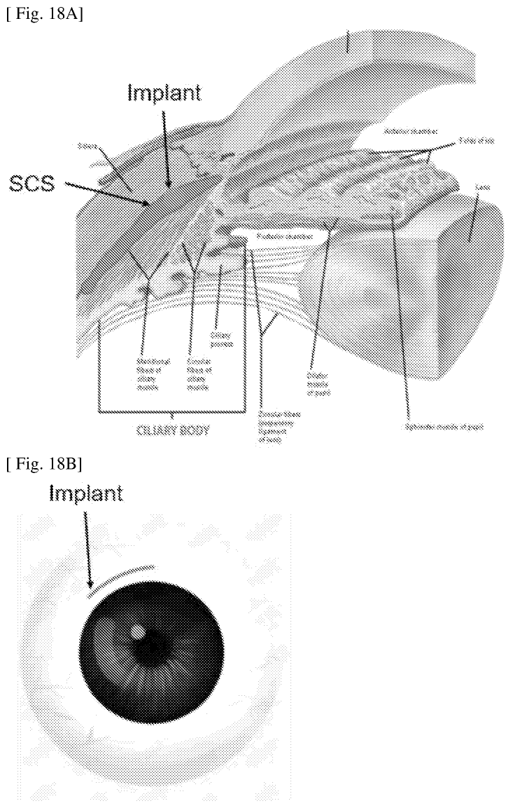

It should be appreciated that any suitable injection device or systems, including but not limited to those described herein in connection with the figures, may be used in a method for drainage from an eye disclosed herein. For instance, an injection device or system shown in FIG. 12A may be used. The injection device or system may be pre-filled with one or more drugs or other substances such as viscoelastic agents. In some embodiments, the injection device or system comprises a syringe barrel comprising a proximal end and a distal end; a floating seal in the syringe barrel; a puncture member such as a needle at the distal end of the syringe barrel, wherein the puncture member is not attached to the floating seal; and an actuation member configured to elastically engage the floating seal via an energy storage member such as a spring or the like and/or another suitable elastic member. In some embodiments, the puncture member comprises a distal end opening configured to form a fluidic communication with a lumen in the syringe barrel containing a flowable composition. In some embodiments, the injection device or system further comprises a stopper in the syringe barrel, between the floating seal and the distal end of the syringe barrel. As shown in FIG. 12A, Step 1, the injection device or system is in an initial state where the distal end opening of the puncture member has not entered a tissue of a subject, and the distance between the actuation member and the floating seal is x₁. In FIG. 12A, Step 2, the distal end opening of the puncture member has entered a relatively dense tissue (e.g., the sclera, anterior chamber angle, or ciliary body), where the distance between the actuation member and the floating seal remains the same (x₁). In FIG. 12A, Step 3, the distal end opening of the puncture member remains in the relatively dense tissue, when the energy storage member is compressed, e.g., by reducing the distance between the actuation member and the floating seal from x₁ to x₂. This way, the energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 12A, Step 4, the puncture member is advanced distally into a less dense tissue, such as an apparent or potential tissue void, cavity, or vessel (for instance, the SCS or the subconjunctival space). In some embodiments, due to the decrease in tissue density, the back pressure on the distal opening of the puncture member becomes less than the pressure of the flowable composition, thereby allowing release of the flowable composition into the less dense tissue, such as the apparent or potential tissue void, cavity, or vessel. As the flowable composition is discharged from the distal end opening of the puncture member, energy in the energy storage member is released, thereby increasing the distance between the actuation member and the floating seal from x₂ to x₃, as shown in FIG. 12A, Step 5. Distal movement of the floating seal in the syringe barrel may be stopped by the stopper, for example, in order to control the volume of the flowable composition delivered into the less dense tissue.

Another example is shown in FIG. 12B, Step 1, where the medical puncture device is in an initial state where the distal end opening of the puncture member has not entered a tissue of a subject, and in FIG. 12B, Step 2, the energy storage member can be compressed, whereas the distal end opening of the puncture member remains outside a tissue and the floating seal is not advanced distally to discharge the flowable composition from the distal end opening. In FIG. 12B, Step 3, the distal end opening of the puncture member has entered a relatively dense tissue (e.g., the sclera, anterior chamber angle, or ciliary body). The energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 12B, Step 4, the distal end opening of the puncture member starts to enter a less dense tissue, such as an apparent or potential tissue void, cavity, or vessel (for instance, the SCS or the subconjunctival space), whereas the energy storage member remains compressed. In FIG. 12B, Step 5, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled. The force applied onto the actuation member may be released as shown in FIG. 12B, Step 6.

Yet another example is shown in FIG. 12C. In some embodiments, the injection device or system comprises a syringe barrel comprising a proximal end and a distal end; a floating seal in the syringe barrel; a puncture member such as a needle at the distal end of the syringe barrel, wherein the puncture member is not attached to the floating seal; and an energy storage member configured to elastically engage the floating seal and the proximal end of the syringe barrel. In some embodiments, the injection device or system further comprises a stopper in the syringe barrel, between the floating seal and the distal end of the syringe barrel. In some embodiments, the medical puncture device comprises a contact member. In FIG. 12C, Step 1, the medical puncture device is in an initial state where the distal end opening of the puncture member is in the contact member which prevents discharge of the flowable composition from the distal end opening. The energy storage member applies a force onto the floating seal, and through the flowable composition and the distal opening of the puncture member, a pressure is in turn applied to the contact member. Due to the density of the contact member, the back pressure on the distal opening of the puncture member prevents leakage of the flowable composition from the syringe barrel. In FIG. 12C, Step 2, the distal end opening of the puncture member has entered a relatively dense tissue (e.g., the sclera, anterior chamber angle, or ciliary body), and the back pressure of the relatively dense tissue on the distal opening prevents leakage of the flowable composition into the tissue. In FIG. 12C, Step 3, the distal end opening of the puncture member starts to enter a less dense tissue, such as an apparent or potential tissue void, cavity, or vessel (for instance, the SCS or the subconjunctival space).

In FIG. 12C, Step 4, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled.

### VII. Method of Improving Injection Precision and Safety

Any adapters described herein can be assembled with a syringe to improve the injection precision and safety of the syringe. In some aspects, provided herein is a method of improving the injection precision and safety of a syringe, comprising:
(1) providing a syringe comprising: a syringe barrel extending from a proximal end to a distal end and forming a lumen extending from the proximal end to the distal end; a push shaft extending from a proximal end to a distal end and forming a seal between the distal end of the push shaft and the syringe barrel; and a needle extending from a proximal end to a distal end comprising an end opening for the fluid to flow from the lumen and through the distal end of the syringe barrel via a needle base;
(2) providing a set of adapters comprising: a contact member extending from a proximal end to a distal end, and a pressing unit comprising a first elastic element;
(3) installing the contact member to the distal end of the needle of the syringe;
(4) installing the pressing unit to the syringe,
wherein the distal end of the contact member is distal to the needle distal end opening, and the distal end of the contact member can directly contact with surface tissues at a target injection site; and
wherein the pressing unit elastically engages the push shaft and/or the syringe barrel via the first elastic element.

In some embodiments, a contact member is installed to the distal end of the needle of a syringe, wherein the distal end of the contact member is distal to the needle distal end opening, and the needle distal end opening is fully within the contact member. In some embodiments, as shown in FIG. 15A, the contact member 25 is a long elastic sheath, and when the contact member is installed, it can touch the needle base of a syringe, therefore build an elastic engagement between the syringe barrel 1 and surface tissues of a target injection site.

In some embodiments, the set of adapters further comprises a second elastic element, wherein the proximal end of the second elastic element, when installed, contacts the needle base. Together with installing the second elastic element, a contact member is also installed to the distal end of the needle of a syringe, wherein the distal end opening of the needle is fully within the contact member. In some embodiments, as shown in FIG. 15B, the contact member 25 is an inelastic block, and the second elastic element 26 (e.g. spring) can build an elastic connection between the proximal end of the contact member and the needle base. In some embodiments, as shown in FIG. 15C, the contact member 25 is an inelastic block, and the second elastic element 26 (e.g., an elastic sheath) can build an elastic connection between the proximal end of the contact member and the needle base. In some embodiments, as shown in FIG. 15D, the contact member 25 comprises an elastic part 25a and an inelastic part 25b, wherein the elastic part 25a is distal to the inelastic part 25b, and the second elastic element 26 (e.g., a spring) can build an elastic connection between the proximal end of the contact member (e.g., the proximal end of the inelastic part of the contact member) and the needle base. In some embodiments, as shown in FIG. 15E, the contact member comprises an elastic part 25a and an inelastic part 25b, wherein the elastic part is distal to the inelastic part, and the second elastic element 26 (e.g., an elastic sheath) can build an elastic connection between the proximal end of the contact member (e.g., the proximal end of the inelastic part of the contact member) and the needle base.

In some embodiments, the set of adapters further comprises a connector, wherein the connector is inelastic, and wherein the proximal end of connector, when installed, contacts the needle base. Together with installing the connector, a contact member is also installed to the distal end of the needle of a syringe, wherein the distal end of the needle is fully within the contact member. In some embodiments, as shown in FIG. 15F, the contact member 25 is elastic sheath, therefore builds an elastic connection between the distal end of the connector 27 and the distal end of the surface tissues at a target injection site.

In some embodiments, a flowable composition is first drawn into the lumen of the syringe, and then the contact member, optionally the second elastic element, and optionally the connector are installed, and then the pressing unit is installed. In some embodiments, the pressing unit is first installed, a flowable composition is then drawn into the lumen of the syringe, and then the contact member, optionally the second elastic element, and optionally the connector are installed.

In some embodiments, as shown in FIG. 16A, the pressing unit 30 comprises a proximal end and a distal end, wherein a first elastic element 31 is connected to the proximal end of the pressing unit. As shown in FIG. 16A, left panel, before being assembled to a syringe, the pressing unit is at an initial state wherein the first elastic element 31 (e.g., spring) is at its static state, without being stretched or compressed. The pressing unit 30 further comprises a pair of stoppers 32 at its distal end, which can inelastically engaged with the syringe barrel of a syringe and block the distal movement of the syringe barrel. As shown in FIG. 16A, right panel, the pressing unit 30 can be assembled to the proximal end of the syringe barrel 1 and push shaft 2, wherein the push shaft 2 is engaged with the pressing unit via the first elastic element 31 (e.g., spring), and the syringe barrel 1 is inelastically engaged with the pressing unit 30 via the pair of stoppers 32, therefore, the push shaft and the syringe barrel is elastically engaged. At this state, the first elastic element 31 is compressed. The compressed first elastic element 31 asserts a force on the push shaft 2, but the contact member at the distal end of the needle prevents discharge of the flowable composition from the distal end opening.

In some embodiments, as shown in FIG. 16B, the pressing unit 30 comprises a proximal end and a distal end connected by the first elastic element 31. As shown in FIG. 16B, left panel, before being assembled to a syringe, the pressing unit 30 is at an initial state wherein the first elastic element 31 (e.g., spring) is at its static state, without being stretched or compressed. The pressing unit 30 further comprises an locking element 33 at its distal end, which can block the distal movement of the syringe barrel. As shown in FIG. 16B, right panel, the pressing unit 30 can be assembled to the proximal end of the syringe barrel 1 and push shaft 2, wherein the push shaft 2 is elastically engaged with the syringe barrel 1 via the first elastic element 31 (e.g., spring), and the first elastic element 31 is stretched. Due to the stretch state of the first elastic element 31, the proximal end of the pressing unit asserts a force on the push shaft 2, but the contact member at the distal end of the needle prevents discharge of the flowable composition from the distal end opening.

As shown in FIG. 17A, Step a-c, the adapters are installed on a syringe and the flowable composition is drawn to the lumen. In FIG. 17A, Step d and e, the syringe with adapters is in an initial state where the distal end opening of the needle is in the contact member, which prevents discharge of the flowable composition from the distal end opening. The first elastic element in the pressing unit (not shown in FIG. 17A) applies a force onto the pushing shaft, further through the flowable composition and the distal opening of the needle, and a pressure is in turn applied to the contact member. Due to the density of the contact member 25, the back pressure on the distal opening of the needle prevents leakage of the flowable composition from the syringe barrel. In FIG. 17A, Step f and g, the distal end opening of the needle has entered a relatively dense tissue A (e.g., the sclera, anterior chamber angle, or ciliary body), and the back pressure of the relatively dense tissue on the distal opening prevents leakage of the flowable composition into the tissue. In the meanwhile, the contact member, optionally the second elastic element, and optionally the connector, can increase the resistance of advancing the needle distally, thereby reducing the risk of overshooting the needle. In FIG. 17A, Step h, the distal end opening of the needle starts to enter a less dense tissue B, such as an apparent or potential tissue void, cavity, or vessel (for instance, the SCS or the subconjunctival space). In FIG. 17A, Step i, due to the decrease in tissue density, the back pressure on the distal opening of the puncture member becomes less than the pressure of the flowable composition, thereby allowing release of the flowable composition into the less dense tissue. Energy stored in the first elastic element is released, as the flowable composition is discharged from the distal end opening of the puncture member. In some embodiments, distal movement of the push shaft in the syringe barrel may be stopped by the stopper to stop the flow of the flowable composition. This way, the volume of the flowable composition delivered into the less dense tissue may be controlled.

In some aspects, set of adapters described herein can also be used to improve the injection precision and safety of other syringes, such as the syringe disclosed in US 2020/0069883 which is incorporated herein by reference for all purposes. The contact member, optionally the second elastic element, and optionally the connector can be installed to the needle of the syringe in the same way as described above. The pressing unit can also be installed to the push shaft of the syringe in the same way as described above.

As shown in FIG. 17B, Step a-c, the adapters are installed on a the syringe disclosed in US 2020/0069883 and the flowable composition is drawn to the lumen. As shown in FIG. 17B, Step d and e, the syringe with adapters is in an initial state where the distal end opening of the needle is in the contact member, which prevents discharge of the flowable composition from the distal end opening. The first elastic element applies a force onto the pushing shaft, and through the flowable composition and the distal opening of the needle, a pressure is in turn applied to the contact member. Due to the density of the contact member 25, the back pressure on the distal opening of the needle prevents leakage of the flowable composition from the syringe barrel. In FIG. 17B, Step f and g, the distal end opening of the needle has entered a relatively dense tissue A (e.g., the sclera, anterior chamber angle, or ciliary body), and the back pressure of the relatively dense tissue on the distal opening prevents leakage of the flowable composition into the tissue. In the meanwhile, the contact member, optionally the second elastic element, and optionally the connector, can increase the resistance of advancing the needle distally, thereby reducing the risk of overshooting the needle. In FIG. 17B, Step h, the distal end opening of the needle starts to enter a less dense tissue B, such as an apparent or potential tissue void, cavity, or vessel (for instance, the SCS or the subconjunctival space). In FIG. 17B, Step i, due to the decrease in tissue density, the back pressure on the distal opening of the needle becomes less than the pressure of the flowable composition, thereby allowing release of the flowable composition into the less dense tissue. Energy stored in the first elastic element is released, pushing the push shaft to move distally, while the contact member blocks the distal movement of the floating seal 3. Therefore, the flowable composition is discharged from the distal end opening of the puncture member. In some embodiments, distal movement of the push shaft in the syringe barrel may be stopped by the stopper to stop the flow of the flowable composition. This way, the volume of the flowable composition delivered into the less dense tissue may be controlled.

The exemplary embodiments and optional implementations of the present disclosure are described in detail above in combination with the figures. However, the present disclosure is not limited to the details described in the embodiments described above. Simple variants can be applied to the embodiments of the present disclosure, all of which are within the scope of the present disclosure.

It should be noted that, each of the technical features described in the embodiments above, when not in conflict, can be combined in any reasonable manner. To avoid unnecessary repetition, the possible combinations are not described separately in the embodiments.

Additionally, the different implementations of the embodiments of the present disclosure can be freely combined. As long as they do not go against the ideas of the present disclosure, they should also be considered part of this disclosure.

1. A pre-filled syringe for injecting a drug composition into an eye, comprising:
a syringe barrel comprising a proximal end and a distal end;
a floating seal in the syringe barrel;
a needle base proximal to the floating seal, wherein the floating seal and the needle base elastically engage each other;
a drug composition, wherein the drug composition is contained in a lumen formed by the floating seal and the distal end of the syringe barrel;
a needle for insertion into the eye, the needle comprising:
(i) a needle proximal end engaging the needle base;
(ii) a needle distal end;
(iii) a needle distal opening;
(iv) a needle body opening between the needle proximal end and the needle distal end, wherein the needle body opening is proximal to the needle distal opening; and
(v) a needle body passageway connecting the needle distal opening and the needle body opening,
wherein the needle base is configured to advance the needle distally toward and/or through the floating seal.

2. The pre-filled syringe of claim 1, wherein the drug composition comprises a formulation of triamcinolone; one or more corticosteroids; one or more tyrosine kinase inhibitors; one or more complement inhibitors; one or more neuroprotective agents; one or more hypoxia factor-inducible inhibitors; one or more adrenergic receptor agonists, gene therapy agents, protein and polypeptide drugs, therapeutic cells or cellular components for cell therapy, or any combination thereof; one or more gel or polymer aqueous solutions; one or more antitumor drugs, herbal medicines, H1 receptor antagonists, mast cell stabilizers, or any combination thereof; one or more antitumor drugs, herbal medicines, H1 receptor antagonists, mast cell stabilizers.

3. The pre-filled syringe of claim 2, wherein the formulation of triamcinolone comprises:
(i) triamcinolone or a pharmaceutically acceptable salt thereof;
(ii) hyaluronic acid or a pharmaceutically acceptable derivative, analog, salt, or solvate thereof;
(iii) one or more buffer agents; and
(iv) one or more tonicity agents.

4. The pre-filled syringe of claim 3, wherein the pharmaceutically acceptably salt of hyaluronic acid comprises a pharmaceutically acceptable metal salt of hyaluronic, preferably an alkali or alkaline salt, more preferably a sodium salt, and particularly preferably a pharmaceutically acceptable salt of hyaluronic with a molecular weight between about 50,000 to about 2,000,000 Daltons.

5. The pre-filled syringe of claim 3, wherein the buffer agents comprise one or more agents selected from the group consisting of acetate buffers, citrate buffers, phosphate buffers, and borate buffers, preferably the buffer agents comprise phosphate buffers, and more preferably the buffer agents comprise disodium hydrogen phosphate, sodium dihydrogen phosphate, or a mixture thereof, wherein the sodium dihydrogen phosphate is in the form of monohydrate and the disodium hydrogen phosphate is in the form of dodecahydrate.

6. The pre-filled syringe of claim 3, wherein the tonicity agents comprise sodium chloride, potassium chloride, or a mixture thereof.

7-11. (canceled)

12. The pre-filled syringe of claim 3, wherein the formulation is prepared by a method comprising:
(a) adding the hyaluronic acid or a pharmaceutically acceptable salt thereof, the buffer agents, the tonicity agents, and optionally a pH adjusting agent in water and mixing;
(b) dispersing triamcinolone in the mixture of step (a);
preferably in step (b), the dispersion is facilitated by stirring; and
preferably the method further comprises (c) wet milling the mixture of step (b).

13. (canceled)

14. The pre-filled syringe of claim 2, wherein the one or more corticosteroids are selected from the group consisting of dexamethasone, triamcinolone acetonide, triamcinolone, triamcinolone acetonide acetate, fluocinolone acetonide, prednisolone, loteprednol, difluprednate, fluorometholone, and any combination thereof.

15. (canceled)

16. The pre-filled syringe of claim 2, wherein the one or more tyrosine kinase inhibitors are selected from the group consisting of axitinib, afatinib, erlotinib, gefitinib, crizotinib, dabrafenib, vemurafenib, dasatanib, imatinib, nilotinib, trametinib, and any combination thereof.

17. (canceled)

18. The pre-filled syringe of claim 2, wherein the one or more complement inhibitors comprise plasma kallikrein inhibitors.

19. (canceled)

20. The pre-filled syringe of claim 2, wherein the one or more neuroprotective agents are selected from the group consisting of cholic acid, chenodeoxycholic acid, deoxycholic acid, glycocholic acid, glycochenodeoxycholic acid, glycodeoxycholic acid, lithocholic acid, taurocholic acid, taurochenodeoxycholic acid, taurodeoxycholic acid, tauroursodeoxycholic acid, ursodeoxycholic acid, and any combination thereof.

21. (canceled)

22. The pre-filled syringe of claim 2, wherein the one or more hypoxia factor-inducible inhibitors are selected from the group consisting of EZN-2698, aminoflavone, camptothecins (e.g., topotecan, EZN-2208, SN38, irinotecan, temsirolimus, everolimus, sirolimus, LY294002, wortmannin, cardiac glycosides, digoxin, ouabain, proscillaridin, 2ME2's, romidepsin (KF228), trichostatin, LW6, acriflavine, echinomycin, anthracyclines (e.g., doxorubicin and daunorubicin), chetomin, bortezomib, and any combination thereof.

23. (canceled)

24. The pre-filled syringe of claim 2, wherein the one or more adrenergic receptor agonists are selected from the group consisting of adrenaline, noradrenaline, isoprenaline, dopamine, phenylephrine, methoxamine, midodrine, oxymetazoline, a-methyldopa, clonidine, brimonidine, dobutamine, salbutamol/albuterol, terbutaline, salmeterol, formoterol, pirbuterol, clenbuterol, and any combination thereof.

25. The pre-filled syringe of claim 2, wherein the one or more gene therapy agents are selected from the group consisting of drugs with AAV2, AAV5, AAV8 and AAV9 vectors as vectors, gene therapy agents with electrotransfer (ET), liposomes and DNA nanoparticles as vectors, and any combination thereof.

26. The pre-filled syringe of claim 2, wherein the one or more protein and polypeptide drugs are selected from the group consisting of anti-VEGF drugs (e.g., bevacizumab, ranibizumab, aflibercept, conbercept, etc.), bispecific antibody drugs (e.g., Faricimab), vasoconstriction agents (e.g., endothelin-1), TNF-α inhibitors (e.g., adalimumab), and any combination thereof.

27. The pre-filled syringe of claim 2, wherein the one or more therapeutic cells or cellular components are selected from the group consisting of stem cells, Treg cells, exosomes, and any combination thereof.

28. (canceled)

29. The pre-filled syringe of claim 2, wherein the one or more gel or polymer aqueous solutions comprise one or more viscoelastic materials.

30. (canceled)

31. (canceled)

32. (canceled)

33. A method of placing a stent in an eye, comprising:
(a) inserting a needle at an injection site of the eye between the sclera and the choroid of the eye;
(b) delivering a flowable composition through the needle to form a suprachoroidal space;
(c) removing the needle from the eye; and
(d) positioning a stent into the suprachoroidal space through the injection site.

34-40. (canceled)

41. A method of improving the injection precision and safety of a syringe, comprising:
(1) providing a syringe comprising: a syringe barrel extending from a proximal end to a distal end and forming a lumen extending from the proximal end to the distal end; a push shaft extending from a proximal end to a distal end and forming a seal between the distal end of the push shaft and the syringe barrel; and a needle extending from a proximal end to a distal end comprising an end opening for the fluid to flow from the lumen and through the distal end of the syringe barrel via a needle base;
(2) providing a set of adapters comprising: a contact member extending from a proximal end to a distal end, and a pressing unit comprising a first elastic element;
(3) installing the contact member to the distal end of the needle of the syringe;
(4) installing the pressing unit to the syringe,
wherein the distal end of the contact member is distal to the needle distal end opening, and the distal end of the contact member can directly contact with surface tissues at a target injection site; and
wherein the pressing unit elastically engages the push shaft and the syringe barrel via the first elastic element.

42-48. (canceled)

49. A set of adapters for a syringe, comprising:
a contact member extending from a proximal end to a distal end;
and a pressing unit comprising a first elastic element;
wherein the syringe comprises a syringe barrel extending from a proximal end to a distal end and forming a lumen extending from the proximal end to the distal end; a push shaft extending from a proximal end to a distal end and forming a seal between the distal end of the push shaft and the syringe barrel; and a needle extending from a proximal end to a distal end comprising an end opening for the fluid to flow from the lumen and through the distal end of the syringe barrel via a needle base;
wherein the contact member can be installed to the needle distal end of the syringe such that the distal end of the contact member is distal to the needle distal end opening, and the distal end of the contact member can directly contact with surface tissues at a target injection site; and
wherein the pressing unit can be installed to the syringe barrel and/or push shaft of the syringe such that the pressing unit elastically engages the push shaft and/or the syringe barrel of the syringe via the first elastic element.

50-56. (canceled)
